SEQUENCE LISTING (32 sequences)

SEQ ID NO: 1 - protein, 559 aa, Homo sapiens
MRLHRLRARLSAVACGLLLLLVRGQGQDSASPIRTTHTGQVLGSLVHVKGANAGVQTFLGIPFAKPPLGPLRFAPPEPPESWSGVRDGTTHPAMCLQDLTAVESEFLSQFNMTFPSDSMSEDCLYLSIYTPAHSHEGSNLPVMVWIHGGALVFGMASLYDGSMLAALENVVVVIIQYRLGVLGFFSTGDKHATGNWGYLDQVAALRWVQQNIAHFGGNPDRVTIFGESAGGTSVSSLVVSPISQGLFHGAIMESGVALLPGLIASSADVISTVVANLSACDQVDSEALVGCLRGKSKEEILAINKPFKMIPGVVDGVFLPRHPQELLASADFQPVPSIVGVNNNEFGWLIPKVMRIYDTQKEMDREASQAALQKMLTLLMLPPTFGDLLREEYIGDNGDPQTLQAQFQEMMADSMFVIPALQVAHFQCSRAPVYFYEFQHQPSWLKNIRPPHMKADHGDELPFVFRSFFGGNYIKFTEEEEQLSRKMMKYWANFARNGNPNGEGLPHWPLFDQEEQYLQLNLQPAVGRALKAHRLQFWKKALPQKIQELEEPEERHTEL

SEQ ID NO: 2 - protein, 561 aa, Mus musculus
MTRNQLHNWLNAGFFGLLLLLIHVQGQDSPEANPIRNTHTGQIQGSLIHVKDTKAGVHTFLGIPFAKPPVGPLRFAPPEAPEPWSGVRDGTAHPAMCLQNLDMLNEAGLPDMKMMLSSFPMSEDCLYLNIYTPAHAHEGSNLPVMVWIHGGALVIGMASMFDGSLLTVNEDLVVVTIQYRLGVLGFFSTGDQHARGNWGYLDQAAALRWVQQNIAHFGGNPDRVTIFGESAGGTSVSSHVVSPMSQGLFHGAIMESGVALLPDLISETSEMVSTTVAKLSGCEAMDSQALVRCLRGKSEAEILAINKVFKMIPAVVDGEFFPRHPKELLASEDFHPVPSIIGVNNDEFGWSIPVVMGSAQMIKGITRENLQAVLKDTAVQMMLPPECSDLLMEEYMGDTEDAQTLQIQFTEMMGDFMFVIPALQVAHFQRSHAPVYFYEFQHPPSYFKDVRPPHVKADHADEIPFVFASFFWGMKLDFTEEEELLSRRMMKYWANFARHGNPNSEGLPYWPVMDHDEQYLQLDIQPAVGRALKAGRLQFWTKTLPQKIQELKASQDKHREL

SEQ ID NO: 3 - protein, 558 aa, Mus musculus
MPLARLPGWLCVVACGLLLLLQHVHGQDSASPIRNTHRGQVRGSFVHVKDTKSGVHAFLGIPFAKPPVGLLRFAPPEDPEPWSGVRDGTSQPAMCLQPDIMNLEDAKEMNLILPPISMSEDCLYLNIYTPTHAQEGSNLPVMVWIHGGGLVVGSASMNDVSKLAATEEIVIVAIQYRLGVLGFFSTGDQHARGNWGYLDQVAALRWVQKNIAYFGGNRDRVTIFGVSAGGTSVSSHILSPMSKGLFHGAIMQSGVALLPDLISDTSEVVYKTVANLSGCEATDSEALIHCLRAKSKQEILAINQVFKMIPAVVDGEFLPKHPQELLTSMDFHPVPSIIGVNTDECGWGVPMFMGLDHIIKNITRETLPAVLKNTAARMMLPPECSHLLVEEYMGDTEDPETLQAQFREMLGDFMFVIPALQVAHFQRSQAPVYFYEFQHLSSFIKHVRPSHVKADHGDDVAFVFGSYLWDMNLDLTEEEELLKRMMMKYWANFARNGNPNSEGLPSWPVLDHDEQYLQLDTQPAVGRALKARRLQFWTKTLPQKIQELKGSQDKHAEL

SEQ ID NO: 4 - protein, 18 aa, Homo sapiens
MKWVTFISLLFLFSSAYS

SEQ ID NO: 5 - protein, 6 aa, Homo sapiens
RGVFRR

SEQ ID NO: 6 - protein, 4 aa, synthetic construct
RGRR

SEQ ID NO: 7 - protein, 6 aa, synthetic construct
RKRKKR

SEQ ID NO: 8 - protein, 4 aa, synthetic construct
RKKR

SEQ ID NO: 9 - protein, 6 aa, synthetic construct
RRRKKR

SEQ ID NO: 10 - protein, 5 aa, synthetic construct
  VARIANT >5: note = The entire sequence of amino acids 1-5 can be repeated one or more times
GSGGS

SEQ ID NO: 11 - protein, 4 aa, synthetic construct
  VARIANT >4: note = The entire sequence of amino acids 1-4 can be repeated one or more times
GGGS

SEQ ID NO: 12 - protein, 5 aa, synthetic construct
  VARIANT >5: note = The entire sequence of amino acids can be repeated one or more times
  VARIANT 1: note = The amino acid can be repeated one or more times
  VARIANT 2: note = The amino acid can be repeated one or more times
  VARIANT 3: note = The amino acid can be repeated one or more times
  VARIANT 4: note = The amino acid can be repeated one or more times
  VARIANT 5: note = The amino acid can be repeated one or more times
GSGSG

SEQ ID NO: 13 - protein, 5 aa, synthetic construct
  VARIANT >5: note = The entire sequence of amino acids can be repeated one or more times
  VARIANT 5: note = The amino acid can be repeated one or more times
GSGGS

SEQ ID NO: 14 - protein, 5 aa, synthetic construct
  VARIANT >5: note = The entire sequence of amino acids can be repeated one or more times
  VARIANT 4: note = The amino acid can be repeated one or more times
GSGSG

SEQ ID NO: 15 - protein, 4 aa, synthetic construct
  VARIANT >4: note = The entire sequence of amino acids can be repeated one or more times
  VARIANT 4: note = The amino acid can be repeated one or more times
GGGS

SEQ ID NO: 16 - protein, 4 aa, synthetic construct
GGSG

SEQ ID NO: 17 - protein, 5 aa, synthetic construct
GGSGG

SEQ ID NO: 18 - protein, 5 aa, synthetic construct
GSGSG

SEQ ID NO: 19 - protein, 5 aa, synthetic construct
GSGGG

SEQ ID NO: 20 - protein, 5 aa, synthetic construct
GGGSG

SEQ ID NO: 21 - protein, 5 aa, synthetic construct
GSSSG

SEQ ID NO: 22 - DNA, 33 bp, synthetic construct
  source 1..33: mol_type = other DNA
gactacaaggatgacgacgataaggggggcggt

SEQ ID NO: 23 - DNA, 12 bp, Mus musculus
  source 1..12: mol_type = genomic DNA
catgcagagctg

SEQ ID NO: 24 - DNA, 12 bp, Mus musculus
  source 1..12: mol_type = genomic DNA
cacagggagctt

SEQ ID NO: 25 - DNA, 58 bp, synthetic construct
  source 1..58: mol_type = other DNA
tgcctttctctccacaggtgtccaggcggccgcgccaccatgccattggctagacttc

SEQ ID NO: 26 - DNA, 55 bp, synthetic construct
  source 1..55: mol_type = other DNA
ccagaggttgattggatccaagcttctacttgtcctgagaacccttgagctcctg

SEQ ID NO: 27 - DNA, 63 bp, synthetic construct
  source 1..63: mol_type = other DNA
tgcctttctctccacaggtgtccaggcggccgcgccaccatgacacggaaccaactacataac

SEQ ID NO: 28 - DNA, 56 bp, synthetic construct
  source 1..56: mol_type = other DNA
ccagaggttgattggatccaagcttctacttgtcctgagaagcctttagctcctgg

SEQ ID NO: 29 - protein, 9 aa, Mus musculus
QKLQELQGR

SEQ ID NO: 30 - protein, 10 aa, Mus musculus
TIQAVLTVPK

SEQ ID NO: 31 - protein, 20 aa, Mus musculus
CQEILSVDCSTNNPAQANLR

SEQ ID NO: 32 - protein, 20 aa, Mus musculus
NNHNMLQEEDLIDEDDILVR

(19) United States
(12) Patent Application Publication
(10) Pub. No.: US 2026/0021165 A1
(43) Pub. Date: Jan. 22, 2026

(54) Extracellular Carboxylesterase 2 (CES2) for the Treatment of Metabolic Disease

(71) Applicant: The Board of Trustees of the Leland Stanford Junior University, Stanford, CA (US)

(72) Inventors: Jonathan Z. Long, Stanford, CA (US); Wei Wei, Stanford, CA (US)

(21) Appl. No.: 18/993,744

(22) PCT Filed: Jul. 12, 2023
(86) PCT No.: PCT/US2023/070030

§ 371 Date: Jan. 13, 2025

Related U.S. Application Data
(60) Provisional application No. 63/390,533, filed on Jul. 19, 2022.

(51) Int. Cl. A61K 38/46; A61P 3/06; C12N 9/18

(52) U.S. Cl. CPC A61K 38/465; A61P 3/06; C12N 9/18; C12Y 301/01001

(57) ABSTRACT

Methods of treating a metabolic disorder in a subject are provided. Aspects of the method include enhancing extracellular Carboxylesterase 2 (CES2) activity in the subject in order to treat the subject for the metabolic disorder. Also provided are compositions for use in practicing the methods.

### CROSS-REFERENCE TO RELATED APPLICATION

This application claims the benefit of U.S. Provisional Patent Application No. 63/390,533, filed Jul. 19, 2022, which application is incorporated herein by reference in its entirety.

### GOVERNMENT RIGHTS

This invention was made with Government support under contract DK130641 awarded by the National Institutes of Health. The Government has certain rights in the invention.

### REFERENCE TO AN ELECTRONIC SEQUENCE LISTING

The contents of the electronic sequence listing (STAN-2006WO_SEQ_LIST.xml; Size: 36,331 bytes; and Date of Creation: Jul. 10, 2023) is herein incorporated by reference in its entirety.

### INTRODUCTION

Metabolic disorders generally refer to a broad array of disorders characterized by defects that interfere with the body's metabolism, the chemical processes by which a body transforms proteins, carbohydrates and fats into energy. Metabolic disorders may include disorders resulting from altered glucose metabolism. Examples of metabolic disorders include obesity, metabolic syndrome, impaired glucose tolerance, and dyslipidemias. A metabolic disorder can also result from a diseased or dysfunctional organ. Diabetes is an example of a metabolic disorder resulting from a diseased and/or dysfunctional organ, the pancreas.

Obesity, which is defined in general terms as an excess of body fat relative to lean body mass, is a serious contributor to increased morbidity and mortality. Obesity, which is most commonly caused by excessive food intake coupled with limited energy expenditure and/or lack of physical exercise, often accompanies various glucose metabolism disorders. Obesity increases the likelihood of an individual developing various diseases, such as diabetes mellitus, hypertension, atherosclerosis, coronary artery disease, gout, rheumatism and arthritis. Obesity is often associated with psychological and medical morbidities, the latter of which includes increased joint problems, vascular diseases such as coronary artery disease, hypertension, stroke, and peripheral vascular disease. Obesity also causes metabolic abnormalities such as insulin resistance and Type II diabetes (non-insulin-dependent diabetes mellitus (NIDDM)), hyperlipidemia, and endothelial dysfunction.

### SUMMARY

Methods of treating a metabolic disorder in a subject are provided. Aspects of the method include enhancing extracellular Carboxylesterase 2 (CES2) activity in the subject in order to treat the subject for the metabolic disorder. Also provided are compositions for use in practicing the methods.

### BRIEF DESCRIPTION OF THE FIGURES

FIG. 1. Study design and overview of exercise training secretomes across 21 cell types in mice. (A) Overview of the study design including viral transduction (AAV9-FLEx-ER-TurbolD, 3*10e11 GC/mouse, intravenously) of 21 cre driver lines (male, N=3/condition/genotype, see Methods) and wildtype C57BL/6 mice (male, N=3/condition), 1-week treadmill running (20 m/min for 60 min per day), secretome labeling (biotin delivered via biotin water (0.5 mg/ml) and via injection (24 mg biotin/ml, intraperitoneally, in a solution of 18:1:1 saline:Kolliphor EL:DMSO, final volume of 200 μl per mouse per day) in the last three days of running), enrichment of biotinylated plasma proteins using streptavidin beads and proteomic analysis. BAT: brown adipose tissue. (B) Volcano plot of adjusted P-values (−log 10) and exercise fold change (log 2) of total 1272 cell type-protein pairs. Adjusted P-values were calculated from moderated t-statistics (see Methods). Black dots indicate exercise-regulated cell type-protein pairs (adjusted P-values <0.05 and exercise fold change >1.5) and gray dots indicate unchanged cell type-protein pairs (adjusted P-values >0.05 or exercise fold change <1.5). (C-F) Relative abundance of exercise training-regulated (C, D and F) and exercise training-unregulated (E) cell type-protein pairs from exercise and sedentary mice. N=3/genotype/condition, mean±SEM. In (C-F), P-values were calculated from two-tailed unpaired t-tests.

FIG. 2. Systematic analysis of exercise training-regulated cell type-protein pairs. (A) Bar graph of exercise training-regulated proteins (black) and unchanged proteins (gray) across 21 cell types. (B) Histogram of increased (gray), decreased (blue) and bidirectionally changed (light gray) secreted proteins after exercise training across 21 cell types. (C-F) Volcano plot of adjusted P-values (−log 10) and exercise fold change (log 2) of indicated example proteins. Black dots indicate exercise training-regulated cell type-protein pairs (adjusted P-values <0.05 and exercise fold change >1.5) and gray dots indicate unchanged cell type-protein pairs (adjusted P-values >0.05 or exercise fold change <1.5). (G) Bubble plot of adjusted P-values (−log 10) and exercise fold change (log 2) of proteins changed in more than 1 cell type after exercise training. Red dots indicate increased proteins after exercise training and blue dots indicated decreased proteins.

FIG. 3. Characterizations of exercise training secretomes from Pdgfra-cre labeled cells. (A) Bar graph of exercise responsiveness scores across cell types. Exercise responsiveness scores of a given cell type were calculated by summarization of the score of individual exercise regulated protein (adjusted P-values <0.05 and exercise fold change >1.5) of that cell type with the following equation: sum(absolute exercise fold change (log 2)×confidence of the change (−log 10(adjusted P-values)))×percent of secretome change (number of exercise training regulated proteins (adjusted P-values <0.05 and exercise fold change >1.5)/total number of secreted proteins of that cell type). See Methods. (B) Volcano plot of adjusted P-values (−log 10) and exercise fold change (log 2) of Pdgfra secretomes. Black dots indicate exercise training-regulated cell type-protein pairs (adjusted P-values <0.05 and exercise fold change >1.5) and gray dots indicate unchanged cell type-protein pairs (adjusted P-values >0.05 or exercise fold change <1.5). (C) Gene ontology analysis of exercise training regulated proteins (adjusted P-values <0.05 and exercise fold change >1.5) from Pdgfra secretomes. Size of bubbles represents P-values (−log 10) of biological processes enrichment and y axis represents gene ratio. (D) Study design of secretome analysis of heterozygous Pdgfra-cre mice (12-week-old male, N=3/condition) injected with 3*10e11 GC/mouse AAV9-FLEx-ER-TurbolD and tamoxifen. Three weeks after tamoxifen delivery, these mice were subjected to acute running (single bout, 20 m/min for 60 min), 3-day or 7-day treadmill running (daily, 20 m/min for 60 min) or being sedentary. Secretome labeling was initiated via injection (24 mg biotin/ml, intraperitoneally, in a solution of 18:1:1 saline:Kolliphor EL:DMSO, final volume of 400 μl per mouse per day) in the last bout of running and biotinylated plasma proteins were enriched using streptavidin beads and analyzed by western blotting (see Methods). (E) Anti-F13A (top), anti-C4BPA (second row), anti-ITIH2 (third row) of eluted biotinylated plasma proteins from streptavidin beads after immune purification. Silver stain of total eluted biotinylated plasma proteins was used as loading control and for quantifications (bottom row). Samples (N=3/condition) were from the experiment described in the legend of FIG. 3D.

FIG. 4. Lactate-induced CES2 secretion in mouse primary hepatocytes. (A) Volcano plot of adjusted P-values (−log 10) and exercise fold change (log 2) of Albumin secretomes. Black dots indicate exercise training-regulated cell type-protein pairs (adjusted P-values <0.05 and exercise fold change >1.5) and gray dots indicate unchanged cell type-protein pairs (adjusted P-values >0.05 or exercise fold change <1.5). (B) Anti-CES2 (bottom) blotting and quantifications of band intensity (top) of immune purified biotinylated plasma proteins from 10-week-old Albumin-cre male mice transduced with 3*10e11 vg AAV9-FLEx-ER-TurbolD virus and exercised on a treadmill for 1-week. N=5/group, mean±SEM. (C-F) Anti-CES2 blotting (bottom) and quantifications of band intensity (D-F) of conditioned medium of primary hepatocytes isolated from 8 to 12-week-old male C57BL/6J mice. Cells were treated with 2 mM indicated organic compounds (C), indicated concentrations of sodium lactate (D), sodium lactate (2 mM) and BFA (5 μg/ml) (E), indicated concentrations of sodium lactate and AR-C155858 (F) for 4 h before analysis. CES2 band intensity was normalized to albumin signal for quantifications. Experiments in each panel contains three biological replicates, mean±SEM. (G) Model of CES2 secretion from cells. Exercise training-inducible rise of extracellular lactate induces release of ER-lumen-resident CES2 from hepatocytes. Functional lactate transporters and ER-Golgi vesicle transport are required for CES2 secretion. P-values for quantifications in this figure were calculated from two-tailed unpaired t-tests.

FIG. 5. Secreted CES2 proteins exhibit anti-obesity, anti-diabetic, and endurance-enhancing effects in mice. (A) Cartoon schematic of conventional ER lumen localized CES2A/C (left) and engineered CES2A/C-AC (right). The C-terminal HXEL sequence was removed from conventional CES2A/C to generate engineered soluble CES2A/C-AC. (B) Cartoon schematic of the AAV constructs driven by the hepatocyte-specific Tbg promoter and study design of HFD feeding experiment. 8 to 10-week-old male C57BL/6 mice were transduced with AAV-Tbg-CES2A-AC or AAV-Tbg-CES2C-AC or AAV-Tbg-GFP (N=10/group, 10e11 GC/mouse, intravenously). 1-week later, mice were placed with HFD feeding for 7 weeks. In the last week, glucose tolerance test and insulin tolerance test were conducted. At the end of this experiment, tissues and blood were harvested and analyzed. (C) Anti-Flag blotting (top) or loading control (bottom) of blood plasma from 16 to 18-week-old male C57BL/6 mice injected with indicated viruses. N=4/condition. (D-I) Body weights over the first 7-week of HFD feeding (D) and food intake (measured weekly) (E), glucose tolerance test (F), insulin tolerance test (G), tissue weights (H) and inguinal white adipose tissue (iWAT), epididymal adipose tissue (eWAT) and brown adipose tissue (BAT) after 48 h of 4% PFA fixation (I) from 16 to 18-week-old male C57BL/6 mice injected with indicated viruses for 8 weeks. N=10/condition, mean±SEM. Samples from (1) were from randomly chosen from mice of each treatment group. (J-L) Maximal running speed (J), total running time (K) and total running distance (L) of 16 to 18-week-old male C57BL/6 mice 8 weeks after being injected with AAV-Tbg-CES2A-AC, AAV-Tbg-CES2C-ΔC or AAV-Tbg-GFP (N=8-10/group, 10e11 GC/mouse, intravenously). Mice were acclimated to the treadmill two days prior to the maximal running tests (10 min at 10 m/min). The maximal running test was performed as previously described⁷⁰⁻⁸⁵ (see Methods). Mean±SEM.P-values for (E), (H) and (J-L) were calculated from two-tailed unpaired t-tests. P-values from (D), (F) and (G) were calculated from two-way ANOVA with post hoc Sidak's multiple comparisons test.

FIG. 6. The anti-obesity effects of soluble CES2 proteins require enzyme activity. (A) Body weights over the first 9-week of HFD feeding of 18 to 20-week-old male C57BL/6 mice injected with indicated viruses (N=10/group, 10e11 GC/mouse, intravenously). Mice were fed with HFD 1-week after viral transduction. Mean±SEM. (B) Untargeted metabolomic measurements of significantly changed features (adjusted P-values <0.05 and fold change >1.5) in blood plasma of 16 to 18-week-old male C57BL/6 mice being transduced with 10e11 AAV-Tbg-CES2A-AC, AAV-Tbg-CES2C-AC or AAV-Tbg-GFP (N=5/group). Mice were placed with HFD feeding for 7 weeks before LC-MS analysis (see Methods). P-values for (A) were calculated from two-way ANOVA with post hoc Sidak's multiple comparisons test.

FIG. 7. Characterization of secretome labeling, running protocol, and secretomes in mice. Related to FIG. 1. (A) mRNA expression of TurbolD in tissues or isolated primary cells, where cre recombinase expression was reported, of wildtype male C57BL/6 mice and 21 male cre driver lines (N=3/genotype, mean±SEM) after AAV9-FLEx-ER-TurbolD viral transduction (3*10e11 GC/mouse, intravenously) (see Methods). (B) Anti-V5 and anti-tubulin (loading control) blotting of murine tissues from wildtype mice C57BL/6 or indicated cre driver lines following transduction of AAV9-FLEx-ER-TurbolD virus. This experiment was repeated with three biological replicates per genotype and similar results were obtained. (C-G) mRNA expression of indicated genes in quadricep muscles (C, D), tissue mass (E) and H&E staining of inguinal white adipose (iWAT) (F) and body weights (G) of wildtype male C57BL/6 mice and 21 male cre driver lines harvested two hours after the final bout of 1-week treadmill running (gray columns) or without exercise training (black columns). N=3/genotype/condition, in total 132 mice, mean±SEM. (H) Daily food intake of wildtype male C57BL/6 mice over 1-week treadling running or without exercise training over 1-week period (N=5/condition, in total 10 mice, mean±SEM). This experiment was repeated twice and similar results were obtained. (I) Cartoon schematic of biotin delivery in mice being subjected to one-week treadmill running or remaining sedentary. P-values for (C-E) and (G-H) were calculated from two-tailed unpaired t-tests.

FIG. 8. Characterizations of Pdgfra+ cells secretomes. Related to FIG. 3. (A) Relative TurbolD mRNA levels from indicated tissues of heterozygous Pdgfra-cre mice (12-week-old male, N=3) injected with 3*10e11 GC/mouse AAV9-FLEx-ER-TurbolD and tamoxifen. Controls samples were from C57BL/6 male mice (10-week-old male, N=3, mean±SEM) injected with 3*10e11 GC/mouse AAV9-FLEx-ER-TurbolD. N=3/group, mean±SEM. (B) Mean Pdgfra gene expression of indicated cell types from Tabula Muris. (C) Quantifications of immuno-staining shown in FIG. 3E. F13A, C4BPA, ITIH2 band intensities were normalized to total silver stain signal in each lane. Each protein from individual exercise groups was compared to sedentary controls. N=3/group, mean±SEM. (D) Volcano plot of adjusted P-values (−log 10) and exercise fold change (log 2) of Pdgfra female secretomes. Black dots indicate exercise training-regulated cell type-protein pairs (adjusted P-values <0.05 and exercise fold change >1.5) and gray dots indicate unchanged cell type-protein pairs (adjusted P-values >0.05 or exercise fold change <1.5). (E) Venn diagram of exercise training-regulated (adjusted P-values <0.05 and exercise fold change >1.5) Pdgfra-cre female and male secretomes. In total, 9 proteins were regulated by exercise training in both secretomes. P-values for (E) were calculated from two-tailed unpaired t-tests.

FIG. 9. Characterizations of lactate induced CES2 secretion in vitro. Related to FIG. 4. (A) Anti-CES2 blotting of recombinant CES2A proteins (top left), recombinant CES2C proteins (top right) and ponceaus stain (bottom). 8 μg of recombinant proteins generated from Expi293 cells were loaded into each lane. (B) Anti-biotin blotting of immune purified biotinylated plasma proteins from 10-week-old Albumin-cre male mice transduced with 3*10e11 vg AAV9-FLEx-ER-TurbolD virus and exercised on a treadmill for 1-week. N=5/group. (C) Circulating lactate of mice after 1-week treadmill running (daily treadmill running, 20 m/min for 60 min) or remaining sedentary (N=5/condition, mean±SEM). Lactate concentration was determined with LC-MS analysis (see Methods). (D) Quantifications of anti-CES2 blotting (N=3 biological replicates, mean±SEM, top panel) and representative anti-Albumin blotting (bottom row) of conditioned medium of primary hepatocytes isolated from 8 to 12-week-old male C57BL/6J mice. Cells were treated with 2 mM indicated organic compounds for 4 h before analysis. CES2 band intensity was normalized to albumin signal in each lane for quantifications. (E) Anti-CES2 stain (second row) and ponceaus stain (bottom row) of cell lysates and anti-Albumin stain (top row) of conditioned medium from primary mouse hepatocytes treated with indicated concentration of sodium lactate. Primary hepatocytes were isolated from 8 to 12-week-old male C57BL/6J mice. Conditioned medium was collected 4 h after the addition of sodium lactate. Experiments were repeated three times, and similar results were obtained. CES2 band intensity was normalized to albumin signal in each lane for quantifications. (F) Anti-Flag blotting of cell lysates and conditioned medium of HEK293T cells transfected with Flag-CES2A construct and treated with indicated concentrations of sodium lactate. Ponceaus staining was used as loading control. 36 h after transfection, cells were washed and replaced with serum-free medium containing indicated concentrations of sodium lactate for 4 h. Cells and conditioned medium were then collected and analyzed. Experiments were repeated three times and similar results were obtained. (G) Anti-CES2 (top row), anti-Albumin (second row) and anti-BHMT (third row) blotting of conditioned medium, and anti-CES2 blotting (fourth row) and ponceaus stain (bottom row) of cell lysates of primary hepatocytes treated with indicated concentrations of sodium lactate and BFA. Primary hepatocytes were isolated from 8 to 12-week-old male C57BL/6J mice. Conditioned medium was collected 4 h after the addition of sodium lactate (2 mM) and BFA (5 μg/ml). Experiments were repeated three times, and similar results were obtained. (H) Anti-Albumin blotting (top row) of conditioned medium and anti-CES2 blotting (second row) and ponceaus stain (bottom row) of cell lysates from primary mouse hepatocytes treated with indicated concentration of sodium lactate and AR-C155858. Primary hepatocytes were isolated from 8 to 12-week-old male C57BL/6J mice. Conditioned medium was collected 4 h after the addition of sodium lactate and AR-C155858. Experiments were repeated three times, and similar results were obtained. CES2 intensity was normalized to albumin signal in each lane for quantifications. P-values for (D) were calculated from two-tailed unpaired t-tests.

FIG. 10. Engineered secreted CES2 in cells and in mice. Related to FIG. 5. (A) Anti-Flag blotting of cell lysates (top left) and conditioned medium (top right) of HEK293T cells transfected with Flag-CES2A, Flag-CES2A-AC, Flag-CES2C, Flag-CES2C-AC constructs. Ponceaus staining was used as loading control (bottom). 36 h after transfection, cells were washed and replaced with serum-free medium for 12 h. Cells and conditioned medium were then collected and analyzed. Experiments were repeated three times and similar results were obtained. (B, E) Relative ester hydrolysis activities of blood plasma (B) and liver lysates (E) from 16 to 18-week-old male C57BL/6 mice 8 weeks after being transduced with indicated viruses. N=9-10/condition, mean±SEM. (C) Anti-Flag (top) of blood plasma (left) and liver lysates (right) from 16 to 18-week-old male C57BL/6 mice 8 weeks after being transduced with indicated viruses. Ponceaus staining was used as loading control (bottom). N=1 in the GFP group and N=3 in the CES2A-ΔC or CES2C-ΔC group. (D) Anti-CES2 (top) and anti-tubulin (loading control, bottom) blotting of liver lysates from 16 to 18-week-old male C57BL/6 mice 8 weeks after being transduced AAV-Tbg-CES2A-ΔC (right) or AAV-Tbg-GFP (left). N=7/condition. (F-O) Body weights (F, G), oxygen consumption (H, I), food intake (J, K), Respiratory Exchange Ratio (RER) (L, M), and movement (N, O) of 14 to 16-week-old male C57BL/6 mice 5 weeks (CES2A-AC) or 3 weeks (CES2C-ΔC) after being transduced with AAV-Tbg-CES2A-ΔC, AAV-Tbg-CES2C-ΔC or AAV-Tbg-GFP. N=8/group, mean±SEM. (P-R) mRNA expression of indicated genes in tibialis anterior muscle (P), soleus muscle (Q) and quadriceps muscle (R). 18 to 20 week-old male C57BL/6 mice were transduced with AAV-Tbg-CES2A-ΔC, AAV-Tbg-CES2C-ΔC or AAV-Tbg-GFP for 9 weeks and fed on high-fat diet for 8 weeks. N=5-6/group, mean±SEM. P-values for (B), (E-G) and (N-O) were calculated from two-tailed unpaired t-tests. P-values for (H-M) were calculated from two-way ANOVA with post hoc Sidak's multiple comparisons test. P-values for (P-R) were calculated from one-way ANOVA with post hoc Sidak's multiple comparisons test.

FIG. 11. Characterizations of elevated soluble CES2 in mice fed with chow diet or with high-fat diet and chronic exercise training. Related to FIG. 5. (A-E) Body weights over the first 7-week of chow diet feeding (A) and food intake (measured weekly) (B), glucose tolerance test (C), insulin tolerance test (D) and tissue weights (E) from 16 to 18-week-old male C57BL/6 mice injected with indicated viruses for 8 weeks. N=8-10/condition, mean±SEM. (F-J) Body weights (F), food intake (measured weekly) (G), glucose tolerance test (H), insulin tolerance test (1) and tissue weights (J) of 17 to 19-week-old male C57BL/6 mice were injected with AAV8-Tbg-CES2A/C-ΔC/GFP viruses via tail vein at a dose of 10e11 GC per mouse. Mice were fed with HFD 1-week after viral transduction and were subjected to treadmill running 4-weeks later (see Methods). N=5 to 6/group, mean±SEM. P-values for (B), (E), (G) and (J) were calculated from two-tailed unpaired t-tests. P-values from (A), (C-D), (F) and (H-I) were calculated from two-way ANOVA with post hoc Sidak's multiple comparisons test.

FIG. 12. Characterizations of elevated soluble wildtype and mutant CES2 in mice. Related to FIG. 6. (A) Enzymatic activities of recombinant CES2A-ΔC (wildtype versus mutant) and CES2C-ΔC (wildtype versus mutant) proteins incubated with 0.5 mM 4-nitrophenyl acetate. The formation of p-nitrophenolate was measured at 405 nm as the indicator of enzymatic activities of CES2-ΔC proteins. N=3/group, mean±SEM. (B) Anti-Flag (top) and blotting of liver lysates from 18 to 20-week-old male C57BL/6 mice 10 weeks after being transduced with indicated virus (N=2/group, 10e11 GC/mouse, intravenously). Mice were fed with HFD 1-week after viral transduction. Ponceaus staining was used as blood plasma loading control (bottom). (C) Food intake (measured weekly) of 18 to 20-week-old male C57BL/6 mice 10 weeks after being transduced with indicated virus (N=10/group, 10e11 GC/mouse, intravenously). Mice were fed with HFD 1-week after viral transduction. Mean±SEM. (D) Schematic of detected peptides for CES2A (top) and CES2C (bottom) protein mapped onto its respective reference sequences with annotated domains indicated below. Pink box indicates detected peptides, grey box indicates undetected region, black box indicates signal peptide, blue box indicates ER retention peptide (HAEL for CES2A and HREL for CES2C), and black line indicates potential N-glycosylation site. P-values for (A) and (C) were calculated from two-tailed unpaired t-tests.

FIG. 13. Exercise-un-regulated cell type-protein pair. Exercise training-unregulated cell type-protein pair from exercise and sedentary mice. N=3/genotype/condition, mean±SEM, P-values were calculated from two-tailed unpaired t-tests.

FIG. 14. Identification of exercise-regulated discordant peptides using peptide correlation analysis. (A) Cartoon schematic of Peptide Correlation Analysis (PeCorA). Peptide Correlation Analysis (PeCorA) uses peptide-level abundance measurements to infer proteoform regulation. Proteoforms that differ in modification state under exercise conditions (top line) will produce similar peptide abundances after tryptic digestion (indicated by black arrows) in both sedentary and exercise conditions, except for the peptide sequences that harbor exercise-induced modifications (blue peptide). PeCorA uses linear models to statistically determine interactions between peptides and treatment groups to reveal quantitative proteoform information, which can be used to infer modification status (blue arrow). (B) Distribution of post-translational modifications seen on the 110 discordant peptides identified by PeCorA processing, as annotated in PhosphoSitePlus and Uniprot. Some modifications could occur on the same peptide. “No known modification” indicates no annotation of a PTM in that peptide sequence, but could also include proteolytic cleavage events that are not easily documented in PTM databases. Peptides with P_{adj}<0.05 as calculated by PeCorA were considered discordant. (C-F) Protein abundance (left), peptide sequence maps (top), and scaled peptide intensity box plots (right) for four examples of discordant peptides from specific cell types. All detected peptides are shown on the sequence maps in dark gray, with the exception of the discordant peptide highlighted in blue. The modification implied by previous database annotation for the discordant peptide is indicated, and the discordant peptide sequence is provided. Signal peptides, known subunits, and disulfide bonds are indicated, but all other potential sites of modification are omitted to only focus on PTMs correlated with discordant peptides. Scaled peptide intensity box plots show the distribution of normalized intensities calculated by PeCorA for all peptides from the sedentary and exercise conditions (gray), with the exception of the discordant peptide from the exercise condition plotted separately (blue). Examples include (C) APOA1 from Vil1+ cells with potential lysine acetylation (Ac, orange) (SEQ ID NO:29); (D) PEDF from Pdgfra+ cells with potential lysine mono-methylation (CH₃, purple) (SEQ ID NO:30); (E) Clusterin from Nr5a1+ cells with potential serine and threonine phosphorylation (P, red) (SEQ ID NO:31); and (F) Complement 4-B from Pdgfra+ cells with potential differential processing between at the interface of the C4 anaphylatoxin and remain C4 alpha chains (SEQ ID NO:32).

FIG. 15. Depicts a schematic of RHBDD1-mediated CES2 release from ER lumen.

FIG. 16. Depicts a Western blot showing that RHBDD1 promotes specific release of CES2.

FIG. 17. Depicts a Western blot showing protease activity of RHBDD1 is required for CES2 release.

FIG. 18. Depicts quantitative PCR data showing CES2-ΔC does not induce canonical thermogenesis programs.

FIG. 19. Depicts a lipolysis assay showing recombinant CES2-ΔC induces lipolysis in differentiated 3T3-L1 cells.

FIG. 20. Depicts a lipolysis assay showing over-expressing CES2-ΔC increases lipolysis of adipose tissue from mice.

### DETAILED DESCRIPTION

Methods of treating a metabolic disorder in a subject are provided. Aspects of the method include enhancing extracellular Carboxylesterase 2 (CES2) activity in the subject in order to treat the subject for the metabolic disorder. Also provided are compositions for use in practicing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, the methods are reviewed first in greater detail, followed by a review of pharmaceutical formulations that find use in embodiments of the methods.

### Methods

As summarized, above, methods of treating a metabolic disorder in a subject are provided. The methods may include administering an effective amount of a CES2 active agent to the subject. As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., obesity. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease. A metabolic disorder may be “treated” if at least one symptom of the metabolic disorder is expected to be or is alleviated, terminated, slowed, or prevented. A metabolic disorder may be also “treated” if recurrence or progression of the metabolic disorder is reduced, slowed, delayed, or prevented.

As summarized above, aspects of the methods include enhancing an extracellular CES2 activity, e.g., a systemic CES2 activity, in the subject in a manner sufficient to treat the metabolic disorder. By “enhancing extracellular CES2 activity” is meant increasing one or more target CES2 activities in the subject. In some instances, the CES2 activity that is enhanced is a systemic CES2 activity, by which is meant a CES2 activity in the circulatory system of the mammal. The magnitude of the increase may vary, where in some instances the magnitude of the increase is 2-fold or greater, such as 5-fold or greater, including 10-fold or greater, e.g., 15-fold or greater, 20-fold or greater, 25-fold or greater (as compared to a suitable control). The CES2 activity that is increased by practice of the methods is an extracellular CES2 mediated process that is beneficial in treating a metabolic disorder. In other words, the CES2 activity that is enhanced is one that results in treatment, e.g., as described herein, of the subject for the metabolic disorder. The target CES2 activity that is enhanced may vary. In some instances, the target CES2 activity is extracellular human CES2 activity, i.e., an activity exhibited by extracellular human CES2 protein. In some instances, the target CES2 activity is extracellular murine CES2 activity, i.e., an activity exhibited by an extracellular mouse CES2 protein, e.g., Ces2a, Ces2c, Ces2e.

The sequence of human CES2 is:
(SEQ ID NO: 01)
MRLHRLRARLSAVACGLLLLLVRGQGQDSASPIRTTHTGQVLGSLVHVKG
ANAGVQTFLGIPFAKPPLGPLRFAPPEPPESWSGVRDGTTHPAMCLQDLT
AVESEFLSQFNMTFPSDSMSEDCLYLSIYTPAHSHEGSNLPVMVWIHGGA
LVFGMASLYDGSMLAALENVVVVIIQYRLGVLGFFSTGDKHATGNWGYLD
QVAALRWVQQNIAHFGGNPDRVTIFGESAGGTSVSSLVVSPISQGLFHGA
IMESGVALLPGLIASSADVISTVVANLSACDQVDSEALVGCLRGKSKEEI
LAINKPFKMIPGVVDGVFLPRHPQELLASADFQPVPSIVGVNNNEFGWLI
PKVMRIYDTQKEMDREASQAALQKMLTLLMLPPTFGDLLREEYIGDNGDP
QTLQAQFQEMMADSMFVIPALQVAHFQCSRAPVYFYEFQHQPSWLKNIRP
PHMKADHGDELPFVFRSFFGGNYIKFTEEEEQLSRKMMKYWANFARNGNP
NGEGLPHWPLFDQEEQYLQLNLQPAVGRALKAHRLQFWKKALPQKIQELE
EPEERHTEL (https://www(dot)uniprot(dot)org/
uniprotkb/O00748/entry)
The sequence of murine Ces2c is:
(SEQ ID NO: 02)
MTRNQLHNWLNAGFFGLLLLLIHVQGQDSPEANPIRNTHTGQIQGSLIHV
KDTKAGVHTFLGIPFAKPPVGPLRFAPPEAPEPWSGVRDGTAHPAMCLQN
LDMLNEAGLPDMKMMLSSFPMSEDCLYLNIYTPAHAHEGSNLPVMVWIHG
GALVIGMASMFDGSLLTVNEDLVVVTIQYRLGVLGFFSTGDQHARGNWGY
LDQAAALRWVQQNIAHFGGNPDRVTIFGESAGGTSVSSHVVSPMSQGLFH
GAIMESGVALLPDLISETSEMVSTTVAKLSGCEAMDSQALVRCLRGKSEA
EILAINKVFKMIPAVVDGEFFPRHPKELLASEDFHPVPSIIGVNNDEFGW
SIPVVMGSAQMIKGITRENLQAVLKDTAVQMMLPPECSDLLMEEYMGDTE
DAQTLQIQFTEMMGDFMFVIPALQVAHFQRSHAPVYFYEFQHPPSYFKDV
RPPHVKADHADEIPFVFASFFWGMKLDFTEEEELLSRRMMKYWANFARHG
NPNSEGLPYWPVMDHDEQYLQLDIQPAVGRALKAGRLQFWTKTLPQKIQE
LKASQDKHREL (https://www(dot)uniprot(dot)org/
uniprotkb/Q91WG0/entry#names_and_taxonomy)
The sequence of murine Ces2a is:
(SEQ ID NO: 03)
MPLARLPGWLCVVACGLLLLLQHVHGQDSASPIRNTHRGQVRGSFVHVKD
TKSGVHAFLGIPFAKPPVGLLRFAPPEDPEPWSGVRDGTSQPAMCLQPDI
MNLEDAKEMNLILPPISMSEDCLYLNIYTPTHAQEGSNLPVMVWIHGGGL
VVGSASMNDVSKLAATEEIVIVAIQYRLGVLGFFSTGDQHARGNWGYLDQ
VAALRWVQKNIAYFGGNRDRVTIFGVSAGGTSVSSHILSPMSKGLFHGAI
MQSGVALLPDLISDTSEVVYKTVANLSGCEATDSEALIHCLRAKSKQEIL
AINQVFKMIPAVVDGEFLPKHPQELLTSMDFHPVPSIIGVNTDECGWGVP
MFMGLDHIIKNITRETLPAVLKNTAARMMLPPECSHLLVEEYMGDTEDPE
TLQAQFREMLGDFMFVIPALQVAHFQRSQAPVYFYEFQHLSSFIKHVRPS
HVKADHGDDVAFVFGSYLWDMNLDLTEEEELLKRMMMKYWANFARNGNPN
SEGLPSWPVLDHDEQYLQLDTQPAVGRALKARRLQFWTKTLPQKIQELKG
SQDKHAEL (https://www(dot)uniprot(dot)org/
uniprotkb/Q8QZR3/entry#sequences)

The target CES2 activity may be provided by a CES2 protein that lacks a c-terminal endoplasmic reticulum (ER) localization domain, e.g., a C-terminal HXEL motif (X=A for Ces2A, and R for Ces2C and CES2).

The target CES2 activity or activities of interest may be enhanced using any convenient protocol. In some instances, the target CES2 activity is enhanced by increasing a systemic level of a CES2 active agent in the mammal. By systemic level is meant the level (e.g., concentration or amount) of the CES2 active agent in the circulatory system of the mammal. The magnitude of the increase may vary, where in some instances the magnitude of the increase is 2-fold or greater, such as 5-fold or greater, including 10-fold or greater, e.g., 15-fold or greater, 20-fold or greater, 25-fold or greater (as compared to a suitable control).

In these embodiments, the systemic level of the CES2 active agent of interest may be increased using any convenient protocol. In some instances, the systemic level is increased by administering a CES2 active agent to the subject. In such instances, the CES2 active agent may vary. CES2 active agents that may be employed in these embodiments of the invention include CES2 polypeptides and nucleic acids encoding the same.

CES2 polypeptides are polypeptides that, upon administration to a subject, exhibit the desired CES2 metabolic disorder treatment activity, e.g., as described above. The term “polypeptide” as used herein refers to full-length proteins as well as portions or fragments thereof which exhibit the desired CES2 activity, e.g., C-Terminal ER localization domain deleted peptides, e.g., as described above. Also included in this term are variations of the naturally occurring proteins, where such variations are homologous or substantially similar to the naturally occurring protein, as described in greater detail below, be the naturally occurring protein the human protein, mouse protein, or protein from some other species which naturally expresses a CES2 protein. In the following description, the term CES2 is used to refer not only to the human form of a CES2 protein, but also to homologs thereof expressed in non-human species.

CES2 polypeptides of interest may vary in terms of amino acid sequence length and molecular weight. In some instances, the CES2 polypeptides range in length from 400 to 800, such as from 450 to 650 and including from about 500 to 600 amino acid residues, and have a projected molecular weight based solely on the number of amino acid residues in the protein and assuming an average molecular weight of 110 Daltons that ranges from 44 to 88 kDa, such as 49.5 to 71.5 kDa, including 55 to 66 kDa, where the actual molecular weight may vary depending on the amount of glycosylation of the protein and the apparent molecular weight may be considerably less because of SDS binding on gels. CES2 polypeptides as described herein may be obtained from naturally sources, e.g., via purification techniques, chemically synthesized or produced using recombinant protocols, as desired.

In some instances, the CES2 polypeptide that is administered to the subject is a human CES2 protein, where the human CES2 protein has an amino acid sequence that comprises a region substantially the same as or identical to the sequence appearing as SEQ ID NO:01. By substantially the same as is meant a protein having a region with a sequence that is 60% or greater, such as 75% or greater, such as 90% or greater and including 98% or greater sequence identity with the sequence of SED ID NO:01, as determined by BLAST using default settings. In some instances, the CES polypeptide that is administered to the subject is a murine Ces2c protein, where the murine Ces2c protein has an amino acid sequence that comprises a region substantially the same as or identical to the sequence appearing as SEQ ID NO:02. By substantially the same as is meant a protein having a region with a sequence that is 60% or greater, such as 75% or greater, such as 90% or greater and including 98% or greater sequence identity with the sequence of SED ID NO:02, as determined by BLAST using default settings. In some instances, the CES2 polypeptide that is administered to the subject is a murine Ces2a protein, where the murine Ces2a protein has an amino acid sequence that comprises a region substantially the same as or identical to the sequence appearing as SEQ ID NO:03. By substantially the same as is meant a protein having a region with a sequence that is 60% or greater, such as 75% or greater, such as 90% or greater and including 98% or greater sequence identity with the sequence of SED ID NO:03, as determined by BLAST using default settings.

In addition to the specific CES2 proteins described above, homologs or proteins (or fragments thereof) from other species, e.g., other animal species, may also be employed in embodiments of the methods, where such homologs or proteins may be from a variety of different types of species, including animals, such as mammals, e.g., rodents, e.g., rats; domestic animals, e.g., horse, cow, dog, cat; etc. By homolog is meant a protein having 35% or more, such as 40% and more and including 60% or more amino acid sequence identity to the specific CES2 proteins as identified in SEQ ID NOS: 01 to 03, where sequence identity is determined using BLAST at default settings.

In addition to the naturally occurring CES2 proteins, e.g., as described above, CES2 polypeptides that vary from the naturally occurring CES2 proteins may also be employed in practicing methods of the invention. Different variations may be present, including but not limited to substitution, insertion and/or deletion mutations, as well as other types of non-amino acid sequence variations, e.g., as illustrated below. CES2 polypeptides that may be employed include proteins having an amino acid sequence encoded by an open reading frame (ORF) of a CES2 gene, including the full length CES2 protein and fragments thereof, such as biologically active fragments and/or fragments corresponding to functional domains; and including fusions of the subject polypeptides to other proteins or parts thereof. Fragments of interest may vary in length, and in some instances are 10 aa or longer, such as 50 aa or longer, and including 100 aa or longer, and in some instances do not exceed 150 aa in length, where a given fragment will have a stretch of amino acids that is substantially the same as or identical to a subsequence found in any of SEQ ID NOS:01 to 03; where the subsequence may vary in length and in some instances is 10 aa or longer, such as 15 aa or longer, up to 50 aa or even longer.

In some instances, CES2 polypeptides employed in methods of invention include or more modifications. Modifications that may be present may vary, and include but are not limited to: amide bond substitutions, amino acid substitutions, including of cysteine residues/analogues, cyclization, pegylation, etc. Examples of modifications that may be found in CES2 polypeptides employed in methods of the invention are now reviewed in greater detail.

In some cases, CES2 polypeptides include one or more linkages other than peptide bonds, e.g., at least two adjacent amino acids are joined via a linkage other than an amide bond. For example, in order to reduce or eliminate undesired proteolysis or other means of degradation, and/or to increase serum stability, and/or to restrict or increase conformational flexibility, one or more amide bonds within the backbone of a CES2 polypeptide can be substituted. In another example, one or more amide linkages (—CO—NH—) in a CES2 polypeptide can be replaced with a linkage which is an isostere of an amide linkage, such as —CH₂NH—, —CH₂S—, —CH₂CH₂—, —CH═CH-(cis and trans), —COCH₂—, —CH(OH)CH₂- or —CH₂SO—. One or more amide linkages in a CES2 polypeptide can also be replaced by, for example, a reduced isostere pseudopeptide bond.

One or more amino acid substitutions can be made in a CES2 polypeptide. The following are non-limiting examples: a) substitution of alkyl-substituted hydrophobic amino acids, including alanine, leucine, isoleucine, valine, norleucine, (S)-2-aminobutyric acid, (S)-cyclohexylalanine or other simple alpha-amino acids substituted by an aliphatic side chain from C₁-C₁₀ carbons including branched, cyclic and straight chain alkyl, alkenyl or alkynyl substitutions; b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine; c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha-methyl-arginine, alpha-methyl-2,3-diaminopropionic acid, alpha-methyl-histidine, alpha-methyl-ornithine where the alkyl group occupies the pro-R position of the alpha-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid; d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids; e) substitution of side chain amide residues, including asparagine, glutamine, and alkyl or aromatic substituted derivatives of asparagine or glutamine; and f) substitution of hydroxyl-containing amino acids, including serine, threonine, homoserine, 2,3-diaminopropionic acid, and alkyl or aromatic substituted derivatives of serine or threonine.

In some cases, a CES2 polypeptide includes one or more naturally occurring non-genetically encoded L-amino acids, synthetic L-amino acids, or D-enantiomers of an amino acid. For example, a CES2 polypeptide can include only D-amino acids. For example, a CES2 polypeptide can include one or more of the following residues: hydroxyproline, β-alanine, o-aminobenzoic acid, m-aminobenzoic acid, β-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, ω-aminohexanoic acid, ω-aminoheptanoic acid, ω-aminooctanoic acid, ω-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

A cysteine residue or a cysteine analog can be introduced into a CES2 polypeptide to provide for linkage to another peptide via a disulfide linkage or to provide for cyclization of the CES2 polypeptide. A CES2 polypeptide can be cyclized. One or more cysteines or cysteine analogs can be introduced into a CES2 polypeptide, where the introduced cysteine or cysteine analog can form a disulfide bond with a second introduced cysteine or cysteine analog. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)ₙ-CO— or —(CH2)ₙ-C₆H₄—CO—) with functional groups which allow for the introduction of a bridge. Some examples are disulfides, disulfide mimetics such as the —(CH2)ₙ— carba bridge, thioacetal, thioether bridges (cystathionine or lanthionine) and bridges containing esters and ethers. In these examples, n can be any integer, but is frequently less than ten.

Other modifications include, for example, an N-alkyl (or aryl) substitution (ψ[CONR]), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

Modifications may be present that provide for improvements in one or more physical properties of the CES2 polypeptide. Improvements of physical properties include, for example, modulating immunogenicity; methods of increasing water solubility, bioavailability, serum half-life, and/or therapeutic half-life; and/or modulating biological activity. Examples of such modifications include, but are not limited to: pegylation, glycosylation (N- and O-linked); polysialylation; albumin fusion molecules comprising serum albumin (e.g., human serum albumin (HSA), cyno serum albumin, or bovine serum albumin (BSA)); albumin binding through, for example a conjugated fatty acid chain (acylation); and Fc-fusion proteins.

Peqylation: The clinical effectiveness of protein therapeutics may be limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of nonproteinaceous polymers, e.g., polyethylene glycol (PEG), polypropylene glycol, or polyoxyalkylenes. This is frequently effected by a linking moiety covalently bound to both the protein and the nonproteinaceous polymer, e.g., a PEG. Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity. In addition to the beneficial effects of pegylation on pharmacokinetic parameters, pegylation itself may enhance activity. PEGs suitable for conjugation to a polypeptide sequence are generally soluble in water at room temperature, and have the general formula R(O—CH₂—CH₂)ₙO-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, and examples are set forth elsewhere herein; by way of example, certain embodiments have molecular weights between 5 kDa and 20 kDa, while other embodiments have molecular weights between 4 kDa and 10 kDa. Pegylated CES2 polypeptides may be conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by any convenient reaction conditions and purification. Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies, such as those known in the art, can be applied herein. PEG may be bound to a polypeptide of the present disclosure via a terminal reactive group (a “spacer”) which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxysuccinylimide. Another activated polyethylene glycol which may be bound to a free amino group is 2,4-bis(0-methoxypolyethyleneglycol)-6-chloro-s-triazine, which may be prepared by reacting polyethylene glycol monomethyl ether with cyanuric chloride. The activated polyethylene glycol which is bound to the free carboxyl group includes polyoxyethylenediamine. Conjugation of one or more of the polypeptide sequences to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH27), and longer reaction time tend to increase the number of PEGs attached. Various means known in the art may be used to terminate the reaction. In some embodiments the reaction is terminated by acidifying the reaction mixture and freezing at, e.g., −20° C. Pegylation of various molecules is discussed in, for example, U.S. Pat. Nos. 5,252,714; 5,643,575; 5,919,455; 5,932,462; and 5,985,263. The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest. This obviates the need for an additional conjugation step during the manufacturing process. Moreover, established molecular biology techniques enable control of the side chain composition of the polypeptide chains, allowing optimization of immunogenicity and manufacturing properties.

Glycosylation: For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present. Glycosylation can dramatically affect the physical properties (e.g., solubility) of polypeptides such as CES2 polypeptides and can also be important in protein stability, secretion, and subcellular localization. Glycosylated polypeptides may also exhibit enhanced stability or may improve one or more pharmacokinetic properties, such as half-life. In addition, solubility improvements can, for example, enable the generation of formulations more suitable for pharmaceutical administration than formulations comprising the non-glycosylated polypeptide. Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants. The polypeptide sequences of the present disclosure may optionally be altered through changes at the nucleic acid level, particularly by mutating the nucleic acid encoding the polypeptide at preselected bases such that codons are generated that will translate into the desired amino acids. Another means of increasing the number of carbohydrate moieties on the polypeptide is by chemical or enzymatic coupling of glycosides to the polypeptide. Removal of carbohydrates may be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases. Dihydrofolate reductase (DHFR)—deficient Chinese Hamster Ovary (CHO) cells are a commonly used host cell for the production of recombinant glycoproteins. These cells do not express the enzyme beta-galactoside alpha-2,6-sialyltransferase and therefore do not add sialic acid in the alpha-2,6 linkage to N-linked oligosaccharides of glycoproteins produced in these cells.

In some embodiments, the polypeptides are non-naturally glycosylated. By non-naturally glycosylated is meant that the polypeptide has a glycosylation pattern, if present, which is not the same as the glycosylation pattern found in the corresponding naturally occurring protein. For example, a human CES2 employed in methods of the invention of this particular embodiment is characterized by having a glycosylation pattern, if glycosylated at all, that differs from that of naturally occurring human CES2. Thus, the non-naturally glycosylated CES2 polypeptides of this embodiment include non-glycosylated CES2 polypeptides, i.e. proteins having no covalently bound glycosyl groups.

Polysialylation: The present disclosure also contemplates the use of polysialylation, the conjugation of polypeptides to the naturally occurring, biodegradable α-(2→8) linked polysialic acid (“PSA”) in order to improve the polypeptides' stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of in vivo activity, and reduction in immunogenicity and antigenicity (see, e.g., G. Gregoriadis et al., Int. J. Pharmaceutics 300(1-2):125-30). As with modifications with other conjugates (e.g., PEG), various techniques for site-specific polysialylation are available (see, e.g., T. Lindhout et al., (2011) PNAS 108(18)7397-7402).

Albumin Fusion: Additional suitable components and molecules for conjugation include albumins such as human serum albumin (HSA), cyno serum albumin, and bovine serum albumin (BSA). Mature HSA, a 585 amino acid polypeptide (˜67 kDa) having a serum half-life of ˜20 days, is primarily responsible for the maintenance of colloidal osmotic blood pressure, blood pH, and transport and distribution of numerous endogenous and exogenous ligands. The protein has three structurally homologous domains (domains 1, 11 and Ill), is almost entirely in the alpha-helical conformation, and is highly stabilized by 17 disulphide bridges. The three primary drug binding regions of albumin are located on each of the three domains within sub-domains IB, IIA and IIIA. Albumin synthesis takes place in the liver, which produces the short-lived, primary product preproalbumin. Thus, the full-length HSA has a signal peptide of 18 amino acids (MKWVTFISLLFLFSSAYS; SEQ ID NO:04) followed by a pro-domain of 6 amino acids (RGVFRR; SEQ ID NO:05); this 24 amino acid residue peptide may be referred to as the pre-pro domain. HSA can be expressed and secreted using its endogenous signal peptide as a pre-pro-domain. Alternatively, HSA can be expressed and secreted using a IgK signal peptide fused to a mature construct. Preproalbumin is rapidly co-translationally cleaved in the endoplasmic reticulum lumen at its amino terminus to produce the stable, 609-amino acid precursor polypeptide, proalbumin. Proalbumin then passes to the Golgi apparatus, where it is converted to the 585 amino acid mature albumin by a furin-dependent amino-terminal cleavage. The primary amino acid sequences, structure, and function of albumins are highly conserved across species, as are the processes of albumin synthesis and secretion. Albumin serum proteins comparable to HSA are found in, for example, cynomolgus monkeys, cows, dogs, rabbits and rats. Of the non-human species, bovine serum albumin (BSA) is the most structurally similar to HSA (see, e.g., Kosa et al., November 2007 J Pharm Sci. 96(11):3117-24). The present disclosure contemplates the use of albumin from non-human species, including, but not limited to, those set forth above, in, for example, the drug development process. According to the present disclosure, albumin may be conjugated to a drug molecule (e.g., a polypeptide described herein) at the carboxyl terminus, the amino terminus, both the carboxyl and amino termini, and internally (see, e.g., U.S. Pat. Nos. 5,876,969 and 7,056,701). In the HSA-CES2 conjugates contemplated by the present disclosure, various forms of albumin may be used, such as albumin secretion pre-sequences and variants thereof, fragments and variants thereof, and HSA variants. Such forms generally possess one or more desired albumin activities. In additional embodiments, the present disclosure involves fusion proteins comprising a polypeptide drug molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker, such as a peptide linker or modified version thereof. Intracellular cleavage may be carried out enzymatically by, for example, furin or caspase. Cells express a low level of these endogenous enzymes, which are capable of cleaving a portion of the fusion molecules intracellularly; thus, some of the polypeptides are secreted from the cell without being conjugated to HSA, while some of the polypeptides are secreted in the form of fusion molecules that comprise HSA. Embodiments of the present disclosure contemplate the use of various furin fusion constructs. For example, constructs may be designed that comprise the sequence RGRR (SEQ ID NO:06), RKRKKR (SEQ ID NO:07), RKKR (SEQ ID NO:08), or RRRKKR (SEQ ID NO:09). The present disclosure also contemplates extra-cellular cleavage (i.e., ex-vivo cleavage) whereby the fusion molecules are secreted from the cell, subjected to purification, and then cleaved. It is understood that the excision may dissociate the entire HSA-linker complex from the mature CES2 polypeptide, or less that the entire HSA-linker complex. As alluded to above, fusion of albumin to one or more polypeptides of the present disclosure can, for example, be achieved by genetic manipulation, such that the nucleic acid coding for HSA, or a fragment thereof, is joined to the nucleic acid coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Transformation is used broadly herein to refer to the genetic alteration of a cell resulting from the direct uptake through the cell membrane, incorporation and expression of exogenous genetic material (exogenous nucleic acid). Transformation occurs naturally in some species of bacteria, but it can also be effected by artificial means in other cells. Furthermore, albumin itself may be modified to extend its circulating half-life. Fusion of the modified albumin to a CES2 polypeptide can be attained by the genetic manipulation techniques described above or by chemical conjugation; the resulting fusion molecule has a half-life that exceeds that of fusions with non-modified albumin. CES2-albumin fusion proteins of interest include those described in U.S. Pat. No. 7,163,805, the disclosure of which is herein incorporated by reference.

Several albumin—binding strategies have been developed as alternatives to direct fusion, including albumin binding through a conjugated fatty acid chain (acylation). Because serum albumin is a transport protein for fatty acids, these natural ligands with albumin—binding activity have been used for half-life extension of small protein therapeutics. For example, insulin determir (LEVEMIR), an approved product for diabetes, comprises a myristyl chain conjugated to a genetically-modified insulin, resulting in a long-acting insulin analog. The present disclosure also contemplates fusion proteins which comprise an albumin binding domain (ABD) polypeptide sequence and the sequence of one or more of the polypeptides described herein. Any ABD polypeptide sequence described in the literature can be a component of the fusion proteins. The components of the fusion proteins can be optionally covalently bonded through a linker, such as those linkers described herein. In some of the embodiments of the present disclosure, the fusion proteins comprise the ABD polypeptide sequence as an N-terminal moiety and the polypeptides described herein as a C-terminal moiety. The present disclosure also contemplates fusion proteins comprising a fragment of an albumin binding polypeptide, which fragment substantially retains albumin binding; or a multimer of albumin binding polypeptides or their fragments comprising at least two albumin binding polypeptides or their fragments as monomer units.

Conjuqation with Other Molecules: Additional suitable components and molecules for conjugation include, for example, thyroglobulin; tetanus toxoid; Diphtheria toxoid; polyamino acids such as poly(D-lysine:D-glutamic acid); VP6 polypeptides of rotaviruses; influenza virus hemagglutinin, influenza virus nucleoprotein; Keyhole Limpet Hemocyanin (KLH); and hepatitis B virus core protein and surface antigen; or any combination of the foregoing. Thus, the present disclosure contemplates conjugation of one or more additional components or molecules at the N- and/or C-terminus of a polypeptide sequence, such as another polypeptide (e.g., a polypeptide having an amino acid sequence heterologous to the subject polypeptide), or a carrier molecule. Thus, an exemplary polypeptide sequence can be provided as a conjugate with another component or molecule. A conjugate modification may result in a polypeptide sequence that retains activity with an additional or complementary function or activity derived from the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disease, disorder or condition as set forth herein (e.g., cancer). A CES2 polypeptide may also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria; and dendritic cells. Such conjugated forms, if desired, can be used to produce antibodies against a polypeptide of the present disclosure. Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes. Purification methods such as cation exchange chromatography may be used to separate conjugates by charge difference, which effectively separates conjugates into their various molecular weights. For example, the cation exchange column can be loaded and then washed with ˜20 mM sodium acetate, pH˜4, and then eluted with a linear (0 M to 0.5 M) NaCl gradient buffered at a pH from about 3 to 5.5, e.g., at pH˜4.5. The content of the fractions obtained by cation exchange chromatography may be identified by molecular weight using conventional methods, for example, mass spectroscopy, SDS-PAGE, or other known methods for separating molecular entities by molecular weight.

Fc-fusion Molecules: In certain embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

Other Modifications: The present disclosure contemplates the use of other modifications, currently known or developed in the future, of CES2 polypeptides to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the polypeptide sequences' characteristics.

Linkers: Linkers and their use have been described above. Any of the foregoing components and molecules used to modify the polypeptide sequences of the present disclosure may optionally be conjugated via a linker. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Exemplary flexible linkers include glycine polymers (G)ₙ, glycine-serine polymers (for example, (GS)ₙ, GSGGSₙ (SEQ ID NO:10), GGGSₙ (SEQ ID NO:11), (GₘSₒ)ₙ, (GₘSₒGₘ)ₙ, (GₘSₒGₘSₒGₘ)ₙ (SEQ ID NO:12), (GSGGSₘ)ₙ (SEQ ID NO:13), (GSGSₘG)ₙ (SEQ ID NO:14) and (GGGSₘ)ₙ (SEQ ID NO:15), and combinations thereof, where m, and o are each independently selected from an integer of at least one), glycine-alanine polymers, alanine-serine polymers, and other flexible linkers. Glycine and glycine-serine polymers are relatively unstructured, and therefore may serve as a neutral tether between components. Exemplary flexible linkers include, but are not limited to GGSG (SEQ ID NO:16), GGSGG (SEQ ID NO:17), GSGSG SEQ ID NO:18), GSGGG (SEQ ID NO:19), GGGSG (SEQ ID NO:20), and GSSSG (SEQ ID NO:21).

In some instances, systemic CES2 polypeptide levels is increased by administering a nucleic acid coding sequence to the subject under conditions sufficient for the coding sequence to be expressed in the subject. Depending on the desired CES2 polypeptide, the nucleic acid coding sequence may vary. Nucleic acids of interest include those encoding the CES2 polypeptides provided above. Specific nucleic acids of interest include, but are not limited to: Human CES2 (NCBI Gene ID: 8824); murine Ces2a (NCBI Gene ID: 102022); and murine Ces2c (NCBI Gene ID: 234671).

By nucleic acid composition is meant a composition comprising a sequence of DNA having an open reading frame that encodes a CES2 polypeptide of interest, i.e., a CES2 coding sequence, and is capable, under appropriate conditions, of being expressed as a CES2 polypeptide. Also encompassed in this term are nucleic acids that are homologous, substantially similar or identical to the specific nucleic acids described above. In addition to the above described specific nucleic acid compositions, also of interest are homologues of the above sequences. In certain embodiments, sequence similarity between homologues is 20% or higher, such as 25% or higher, and including 30%, 35%, 40%, 50%, 60%, 70% or higher, including 75%, 80%, 85%, 90% and 95% or higher. Sequence similarity is calculated based on a reference sequence, which may be a subset of a larger sequence, such as a conserved motif, coding region, flanking region, etc. A reference sequence may be 18 nt long or longer, such as 30 nt long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17). Of particular interest in certain embodiments are nucleic acids of substantially the same length as specific CES2 encoding nucleic acids mentioned above, where by substantially the same length is meant that any difference in length does not exceed about 20 number %, usually does not exceed about 10 number % and more usually does not exceed about 5 number %; and have sequence identity to any of these sequences of at 90% or greater, such as 95% or greater and including 99% or greater over the entire length of the nucleic acid. In some embodiments, the nucleic acids have a sequence that is substantially similar or identical to the above specific sequences. By substantially similar is meant that sequence identity is 60% or greater, such as 75% or greater and including 80, 85, 90, or even 95% or greater. Nucleic acids of interest also include nucleic acids that encode the proteins encoded by the above described nucleic acids, but differ in sequence from the above described nucleic acids due to the degeneracy of the genetic code.

Nucleic acids as described herein may be present in a vector. Various vectors (e.g., viral vectors, bacterial vectors, or vectors capable of replication in eukaryotic and prokaryotic hosts) can be used in accordance with the present invention. Numerous vectors which can replicate in eukaryotic and prokaryotic hosts are known in the art and are commercially available. In some instances, such vectors used in accordance with the invention are composed of a bacterial origin of replication and a eukaryotic promoter operably linked to a DNA of interest.

Viral vectors used in accordance with the invention may be composed of a viral particle derived from a naturally-occurring virus which has been genetically altered to render the virus replication-defective and to express a recombinant gene of interest in accordance with the invention. Once the virus delivers its genetic material to a cell, it does not generate additional infectious virus but does introduce exogenous recombinant genes into the cell, preferably into the genome of the cell. Numerous viral vectors are well known in the art, including, for example, retrovirus, adenovirus, adeno-associated virus, herpes simplex virus (HSV), cytomegalovirus (CMV), vaccinia and poliovirus vectors.

The DNA of interest may be administered using a non-viral vector, for example, as a DNA- or RNA-liposome complex formulation. Such complexes comprise a mixture of lipids which bind to genetic material (DNA or RNA), providing a hydrophobic coat which allows the genetic material to be delivered into cells. Liposomes which can be used in accordance with the invention include DOPE (dioleyl phosphatidyl ethanol amine), CUDMEDA (N-(5-cholestrum-3-.beta.-ol 3-urethanyl)-N′,N′-dimethylethylene diamine). When the DNA of interest is introduced using a liposome, in some instances one first determines in vitro the optimal values for the DNA: lipid ratios and the absolute concentrations of DNA and lipid as a function of cell death and transformation efficiency for the particular type of cell to be transformed. These values can then be used in or extrapolated for use in in vivo transformation. The in vitro determinations of these values can be readily carried out using techniques which are well known in the art.

Other non-viral vectors may also be used in accordance with the present invention. These include chemical formulations of DNA or RNA coupled to a carrier molecule (e.g., an antibody or a receptor ligand) which facilitates delivery to host cells for the purpose of altering the biological properties of the host cells. By the term “chemical formulations” is meant modifications of nucleic acids to allow coupling of the nucleic acid compounds to a carrier molecule such as a protein or lipid, or derivative thereof. Exemplary protein carrier molecules include antibodies specific to the cells of a targeted secretory gland or receptor ligands, i.e., molecules capable of interacting with receptors associated with a cell of a targeted secretory gland.

DNA constructs may include a promoter to facilitate expression of the DNA of interest within a target cell, such as a strong, eukaryotic promoter. Exemplary eukaryotic promoters include promoters from cytomegalovirus (CMV), mouse mammary tumor virus (MMTV), Rous sarcoma virus (RSV), and adenovirus. More specifically, exemplary promoters include the promoter from the immediate early gene of human CMV (Boshart et al., Cell 41:521-530, 1985) and the promoter from the long terminal repeat (LTR) of RSV (Gorman et al., Proc. Natl. Acad. Sci. USA 79:6777-6781, 1982).

Instead of administration of a CES2 polypeptide, e.g., as described above, the level of systemic CES2 active agent in the subject may be enhanced by stimulating endogenous production and/or release of a CES2 polypeptide in vivo.

Also of interest are potentiators of CES2 activity. By CES2 potentiator is meant an agent or combination of agents that work to increase the desirable extracellular CES2 activity of endogenous CES2 polypeptides present in the subject being treated. The magnitude of the increase may vary, where in some instances the magnitude of the increase is 2-fold or greater, such as 5-fold or greater, including 10-fold or greater, e.g., 15-fold or greater, 20-fold or greater, 25-fold or greater (as compared to a suitable control). CES2 potentiators of interest may work through a variety of different mechanisms, e.g., by enhancing the binding interaction between a CES2 polypeptide and a desired target; by increasing the bioavailability of the endogenous pool, e.g., by sequestering undesirable competitive binding targets, etc.

In yet other embodiments, the agent is a small molecule agent that exhibits the desired extracellular CES2 activity. Naturally occurring or synthetic small molecule compounds of interest include numerous chemical classes, such as organic molecules, e.g., small organic compounds having a molecular weight of more than 50 and less than about 2,500 Daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below.

Any suitable amount of the CES2 active agent may be administered. In some instances, an amount effective to treat a metabolic disease or associated condition, e.g., an effective amount, is administered. In some instances, the effective amount includes an amount of the CES2 active agent that when administered produces a plasma concentration of the CES2 activity comparable (e.g., equivalent) to that observed in the subject during or after physical activity. For example, the effective amount may be equivalent to the amount of extracellular CES2 protein present in the body, e.g., blood plasma, of the subject during or after physical activity. In some instances, the effective amount includes an amount of the CES2 active agent that when administered produces a plasma concentration of the extracellular CES2 protein comparable (e.g., equivalent) to that observed in the subject during or after a recovery period after physical activity (e.g., a period of time after physical activity that ranges from 1 minute to 5 hours including, e.g., from 1 minute to 4 hours, from 1 minute to 3 hours, from 1 minute to 2 hours, from 1 minute to 1 hour, from 1 minute to 30 minutes, or from 1 minute to 15 minutes). For example, the effective amount may be equivalent to the amount present in the body, e.g., plasma, during or after a recovery period after physical activity. Physical activity may include any amount of physical activity over a period of time. In some instances, physical activity includes an amount of physical activity performed over a period of time ranging from 1 minute to 2 hours including, e.g., from 1 minute to 1 hour, or from 1 minute to 30 minutes. Physical activity may include, e.g., any activity that raises the heart rate above a resting heart rate, physical movement, exercise, any activity performed to maintain or achieve physical fitness, etc. In some instances, the effective amount includes a single unit dose of the CES2 active agent. In some instances, the effective amount includes one or more unit doses of the CES2 active agent including, e.g., two or more doses, three or more doses, four or more doses, etc. In some instances, a single dose is administered. In some instances, multiple doses, e.g., two or more, three or more, etc., are administered. The effective amount may range from 1 mg/kg to 500 mg/kg including, e.g., from 1 mg/kg to 400 mg/kg, from 1 mg/kg to 300 mg/kg, from 1 mg/kg to 200 mg/kg, from 1 mg/kg to 100 mg/kg.

In some instances, the methods include administering an amount of the CES2 active agent effective to induce a physical activity associated outcome in a subject (e.g., effective to cause the subject to experience a physical activity associated outcome). By “physical activity associated outcome” is meant an outcome, change, or effect (e.g., biological, physical, and/or chemical) equivalent to that induced in the subject by physical activity (e.g., equivalent in magnitude and longevity). Physical activity associated outcomes of interest include, but are not limited to, weight loss, prevention or treatment of metabolic disorders and associated conditions, improved glucose homeostasis, improved thinking or cognition, mood improvement, reduction in the severity of mood disorders (e.g., anxiety and depression), prevention of or slowing the progress of neurodegenerative diseases (e.g., dementia), improvement in sleep, lower risk of cancer, among others. For example, the methods may include administering an amount of the CES2 active agent effective to induce a reduction in body weight in the subject equivalent to what would be induced by physical activity. In another example, the methods may include administering an amount of the CES2 active agent effective to induce an improvement in glucose homeostasis in the subject equivalent to what would be induced by physical activity. In another example, the methods may include administering an amount of the CES2 active agent effective to induce a mood improvement in the subject equivalent to what would be induced by physical activity. In yet another example, the methods may include administering an amount of the CES2 active agent effective to induce a reduction in the severity of a mood disorder in the subject equivalent to what would be induced by physical activity. In yet another example, the methods may include administering an amount of the CES2 active agent effective to induce a reduction in the severity of a neurodegenerative disorder (e.g., prevention of the development of the disorder or slowing of the progression of the disorder) in the subject equivalent to what would be induced by physical activity. In some embodiments, the methods include administering an amount of the CES2 active agent effective to treat a nervous system disorder and/or associated conditions. The nervous system disorder may include, e.g., mood or psychiatric disorders (e.g., anxiety, depression, bipolar disorder, seasonal affective disorder, etc.) or a neurodegenerative disease (e.g., dementia, Alzheimer's disease, Parkinson's disease, etc.). The effective amount may be any of the amounts described herein.

In some instances, the CES2 active agent may be administered according to a dosing schedule. In some instances, the effective amount is administered once to the subject. In some instances, the effective amount is administered once a day to the subject. In some instances, the effective amount is administered multiple times a day to the subject. In some instances, the effective amount is administered once a day over a period of time ranging from 1 day to 60 days, e.g., from 1 day to 10 days, from 1 day to 7 days, from 1 day to 5 days, or from 1 day to 3 days. In some instances, the effective amount is administered from 1 time to 5 times per day including, e.g., 1 time to 3 times per day, 2 times to 5 times per day, or 3 times to 5 times per day, over a period of time ranging from 1 day to 14 days, e.g., from 1 day to 10 days, from 1 day to 7 days, from 1 day to 5 days, or from 1 day to 3 days.

In certain embodiments, the methods include administering the CES2 active agent in combination with one or more therapies for treating a metabolic disorder and/or associated condition, e.g., obesity. In some instances, the methods include administering the CES2 active agent in combination with an active agent (or a combination of one or more active agents) for treating the metabolic disorder. In some instances, the methods include administering the CES2 active agent in combination with an active agent for treating obesity. Active agents of interest include, but are not limited to, orlistat, lorcaserin, phentermine-topiramate, naltrexone-bupropion, liraglutide, phentermine, benzphetamine, diethylpropion, phendimetrazine, among others. In some instances, the therapies that may be used in combination with the CES2 active agent to treat the metabolic disorder (e.g., obesity) include any one of or a combination of any of the following therapies: physical activity, a dietary plan (e.g., a low fat diet, low calorie diet, intermittent fasting, etc.), use of a weight loss device, and surgical intervention (e.g., bariatric surgery). Weight loss devices may include, e.g., an electrical stimulation system (e.g., a device that blocks nerve activity between the stomach and brain), a gastric balloon system (e.g., one or more balloons placed in the stomach), and/or a gastric emptying system (e.g., a pump and tube to drain food from the stomach after a meal).

In some instances, the one or more therapies include a small molecule agent. Naturally occurring or synthetic small molecule compounds of interest include numerous chemical classes, such as organic molecules, e.g., small organic compounds having a molecular weight of more than 50 and less than about 2,500 Daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols.

In some instances, the one or more therapies include a protein or a fragment thereof or a protein complex. In some embodiments, the one or more therapies include an antibody binding agent or derivative thereof. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. In some cases, the one or more therapies include an enzyme or enzyme complex. In some cases, the one or more therapies includes a phosphorylating enzyme, e.g., a kinase. In some cases, the one or more therapies includes a complex including a guide RNA and a CRISPR effector protein, e.g., Cas9, used for targeted cleavage of a nucleic acid.

In some embodiments, the one or more therapies includes a nucleic acid. The nucleic acids may include DNA or RNA molecules. In certain embodiments, the nucleic acids modulate, e.g., inhibit or reduce, the activity of a gene or protein, e.g., by reducing or downregulating the expression of the gene. The nucleic acid may be a single stranded or double-stranded and may include modified or unmodified nucleotides or non-nucleotides or various mixtures and combinations thereof. In some cases, the one or more therapies includes intracellular gene silencing molecules by way of RNA splicing and molecules that provide an antisense oligonucleotide effect or an RNA interference (RNAi) effect useful for inhibiting gene function. In some cases, gene silencing molecules, such as, e.g., antisense RNA, short temporary RNA (stRNA), double-stranded RNA (dsRNA), small interfering RNA (siRNA), short hairpin RNA (shRNA), microRNA (miRNA), tiny non-coding RNA (tncRNA), snRNA, snoRNA, and other RNAi-like small RNA constructs, may be used to target a protein-coding as well as non-protein-coding genes. In some case, the nucleic acids include aptamers (e.g., spiegelmers). In some cases, the nucleic acids include antisense compounds. In some cases, the nucleic acids include molecules which may be utilized in RNA interference (RNAi) such as double stranded RNA including small interfering RNA (siRNA), locked nucleic acid (LNA) inhibitors, peptide nucleic acid (PNA) inhibitors, etc.

The CES2 active agent may be administered by any suitable means. As used herein, the term “administering” includes in vivo administration as well as direct administration to tissues ex vivo. Generally, administration is, for example, oral, buccal, parenteral (e.g., intravenous, intraarterial, subcutaneous), intraperitoneal (i.e., into the body cavity), topically, e.g., by inhalation or aeration (i.e., through the mouth or nose), or rectally systemic (i.e., affecting the entire body). A composition may be administered in dosage unit formulations containing conventional non-toxic pharmaceutically acceptable carriers, adjuvants, and vehicles as desired. The term “topically” may include injection, insertion, implantation, topical application, or parenteral application.

In some embodiments, the CES2 active agent is administered in a pharmaceutical formulation or as a pharmaceutically acceptable composition in which a CES2 active agent may be mixed with one or more carriers, thickeners, diluents, buffers, preservatives, surface active agents, excipients and the like. Pharmaceutical compositions may also include one or more additional active ingredients such as antimicrobial agents, anti-inflammatory agents, anesthetics, and the like in addition to the CES2 active agent. In some cases, the CES2 active agent composition includes, e.g., a derivative or analog of CES2 active agent. “Derivatives” include pharmaceutically acceptable salts and chemically modified agents. “Analogs” include a chemical compound that is structurally similar to another but differs slightly in composition (as in the replacement of one atom by an atom of a different element or in the presence of a particular functional group, or the replacement of one functional group by another functional group). Thus, an analog may be a compound that is similar or comparable in function and appearance, but not in structure or origin to the reference compound. The pharmaceutical compositions may be administered by any route commonly used to administer pharmaceutical compositions. For example, administration may be done topically (including ophthalmically, vaginally, rectally, intranasally), orally, by inhalation, or parenterally, for example by intravenous drip or subcutaneous, intraperitoneal or intramuscular injection. The pharmaceutical composition including the CES2 active agent may be stored at any suitable temperature. In some cases, the CES2 active agent composition is stored at temperatures ranging from 1° C. to 30° C., from 2° C. to 27° C., or from 5° C. to 25° C. The CES2 active agent composition may be stored in any suitable container, as described in detail below.

The metabolic disorder treated by the subject methods may vary. By “metabolic disorder,” “metabolic condition,” “metabolic disease,” “metabolic disease-associated condition,” or “metabolic disorder-associated condition” is meant a disorder or condition relating to abnormality of metabolism. In some instances, a “metabolic disorder” refers to any disorder associated with or aggravated by impaired or altered glucose regulation or glycemic control, such as, for example, insulin resistance. Such disorders include, but are not limited to, diabetes, hyperglycemia, obesity, etc. Metabolic disorders and conditions associated with metabolic disorders that can be treated according to the methods described herein include but are not limited to overweight, obesity, hyperphagia, diabetes (inclusive of type 1 diabetes and type 2 diabetes), type 2 diabetes, impaired glucose tolerance, insulin resistance, hyperinsulinemia, dyslipidemia, hypertension, metabolic syndrome. The disorder treated by the subject methods may also be obesity and metabolic syndrome associated disorders, such as but not limited to, meningioma, adenocarcinoma, multiple myeloma, kidney cancer, endometrium cancer, ovarian cancer, colorectal cancer, pancreatic cancer, stomach cancer, gallbladder cancer, liver cancer, breast cancer, thyroid cancer, and any other obesity associated cancers. Including osteoarthritis, stroke, gallbladder disease, chronic kidney disease, and coronary artery disease. Including mental disorders such as clinical depression and anxiety, bipolar disorder, panic disorder, and agoraphobia. Metabolic disorders that can be treated according to the methods described herein can be included in embodiments individually or in any combination.

In some instances, the metabolic disorder or metabolic disorder-associated condition is obesity. The term “obesity” refers to a condition characterized by an excess of body fat. The operational definition of obesity may be based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m² to less than 25 kg/m².

In some instances, the metabolic disorder is an obesity-related metabolic disorder. The term “obesity-related disorders” encompasses disorders that are associated with, caused by, or result from obesity. Examples of obesity-related disorders include overeating and bulimia, diabetes, hypertension, elevated plasma insulin concentrations and insulin resistance, dyslipidemia, hyperlipidemia, breast, prostate, endometrial and colon cancer, heart disease, cardiovascular disorders, abnormal heart rhythms and arrhythmias, myocardial infarction, congestive heart failure, coronary heart disease, angina pectoris, cerebral infarction, cerebral thrombosis and transient ischemic attack. Other examples include pathological conditions showing reduced metabolic activity or a decrease in resting energy expenditure as a percentage of total fat-free mass. Further examples of obesity-related disorders include metabolic syndrome, also known as syndrome X, insulin resistance syndrome, type II diabetes, impaired fasting glucose, impaired glucose tolerance, inflammation, such as systemic inflammation of the vasculature, atherosclerosis, hypercholesterolemia, hyperuricaemia, as well as secondary outcomes of obesity such as left ventricular hypertrophy. Obesity-related metabolic disorders may further include, e.g., hypertension, osteoarthritis, Type II diabetes mellitus, increased blood pressure, stroke, and heart disease. Obesity-related disorders also include the liver abnormalities associated with obesity such as steatosis or non-alcoholic fatty liver disease (NAFLD) a rising cause of cirrhosis associated to obesity and metabolic syndrome. Indeed, NAFLD can present as simple steatosis or evolve towards inflammation and steatohepatitis (NASH), with a 20% risk of cirrhosis after 20 years. “Dyslipidemia” is a major risk factor for coronary heart disease (CHD). Low plasma levels of high density lipoprotein (HDL) cholesterol with either normal or elevated levels of low density (LDL) cholesterol is a significant risk factor for developing atherosclerosis and associated coronary artery disease in humans. Dyslipidemia is often associated with obesity. Additional obesity related disorders are described in, e.g., U.S. Pat. No. 8,394,969, the disclosure of which is incorporated herein by reference in its entirety.

In some instances, the metabolic disorder is diabetes. “Diabetes” refers to a group of metabolic diseases characterized by high blood sugar (glucose) levels which result from defects in insulin secretion or action, or both. Diabetes is classified according to the types of disease into insulin dependent diabetes (IDDM; type I diabetes) and non-insulin dependent diabetes (NIDDM; type II diabetes). “Type 2 diabetes” refers to one of the two major types of diabetes, the type in which the beta cells of the pancreas produce insulin, at least in the early stages of the disease, but the body is unable to use it effectively because the cells of the body are resistant to the action of insulin. In later stages of the disease the beta cells may stop producing insulin. Type 2 diabetes is also known as insulin-resistant diabetes, non-insulin dependent diabetes and adult-onset diabetes. “Type I diabetes” refers to a condition that results from an autoimmune-mediated destruction of pancreatic s cells with consequent loss of insulin production, which results in hyperglycemia. Type I diabetics require insulin replacement therapy to ensure survival. The term “diabetic disorders” may refer to complications due to diabetes. For example, complications such as retinopathy, nephropathy and neuropathy develop with angiopathy as a prime factor in diabetic individuals.

Treatment may result in various outcomes. In certain embodiments, treatment of obesity and obesity-related disorders refers to the administration of the CES2 active agent as described herein to reduce or maintain the body weight of an obese subject. One outcome of treatment may be reducing the body weight of an obese subject relative to that subject's body weight immediately before the administration of the compounds or combinations as described herein. Another outcome of treatment may be preventing regain of body weight previously lost as a result of diet, exercise, or pharmacotherapy and preventing weight gain from cessation of smoking. Another outcome of treatment may be decreasing the occurrence of and/or the severity of obesity-related diseases. Yet another outcome of treatment may be decreasing the risk of developing diabetes in an overweight or obese subject. The treatment may result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate. The treatment may result in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

In some instances, the methods prevent the development of obesity or obesity-related disorders in a subject. Prevention of obesity and obesity-related disorders refers to the administration of the CES2 active agent to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

In certain embodiments, the methods reduce food intake of the subject, e.g., during and/or after treatment. By “food intake” is meant the amount of food consumed by the subject. In some instances, food intake is measured in kcal over a period of time, e.g., kcal/day. In some instances, the food intake is cumulative food intake over a period of time ranging from 1 day to 14 days, e.g., from 1 day to 10 days, from 1 day to 7 days, from 1 day to 5 days, or from 1 day to 3 days. In some instances, the food intake is average daily food intake, e.g., over a period of time ranging from 1 day to 14 days, e.g., from 1 day to 10 days, from 1 day to 7 days, from 1 day to 5 days, or from 1 day to 3 days. In some instances, the methods reduce food intake by the subject compared to (e.g., relative to) a control. In some instances, the methods reduce food intake by the subject compared to the food intake of the subject before treatment. In some instances, cumulative food intake is reduced by 10% to 90% including, e.g., by 10% to 80%, by 10% to 70%, by 10% to 60%, by 10% to 50%, by 10% to 40%, by 10% to 30%, or by 10% to 20%. In some instances, average daily food intake is reduced by 10% to 90% including, e.g., by 10% to 80%, by 10% to 70%, by 10% to 60%, by 10% to 50%, by 10% to 40%, by 10% to 30%, or by 10% to 20%.

In certain embodiments, the methods reduce the body weight of the subject, e.g., during and/or after treatment. In some instances, the methods reduce the body weight of the subject compared to (e.g., relative to) a control. In some instances, the methods reduce the body weight of the subject compared to the body weight of the subject before treatment. In some instances, the methods reduce the average body weight of the subject over a period of time ranging, e.g., from 1 day to 14 days, e.g., from 1 day to 10 days, from 1 day to 7 days, from 1 day to 5 days, or from 1 day to 3 days. In some instances, the methods reduce the average body weight of the subject compared to a control. In some instances, the methods reduce the average body weight of the subject compared to the average body weight of the subject before treatment. In some instances, body weight is reduced by 1% to 50% including, e.g., by 1% to 40%, by 1% to 30%, by 1% to 20%, or by 1% to 10%. In some instances, average body weight is reduced by 1% to 50% including, e.g., by 1% to 40%, by 1% to 30%, by 1% to 20%, or by 1% to 10%.

In certain embodiments, the methods improve glucose regulation in a subject, e.g., during and/or after treatment. For example, the methods may improve the body's ability to regulate glucose. The term “glucose regulation” or “regulation of glucose metabolism” as used herein refer to processes by which a cell, tissue, organ, organ system, or whole organism maintains glucose homeostasis by altering, e.g., increasing or decreasing, specific processes of glucose metabolism. Glucose metabolism or glucose metabolic processes encompass processes involving glucose synthesis, processing, transport, uptake, utilization, or storage, and includes gluconeogenesis and glycolysis. Specific aspects of glucose metabolism and regulation include expression of glucose transporters or enzymes which facilitate movement of glucose across a cell membrane and retention or secretion of glucose by a cell; alteration in expression and/or activity of enzymes involved in glucose utilization or formation, including, e.g., glycolytic and gluconeogenic enzymes; and alteration of glucose distribution within body or culture fluids, including, e.g., interstitial (i.e. extracellular) and intracellular fluids, blood, urine, and the like.

In some embodiments, the methods improve glucose homeostasis in the subject, e.g., during and/or after treatment. The term “glucose homeostasis” refers to maintenance of normal glucose levels, e.g., normal blood glucose levels, in an organism. In some instances, the methods improve glucose homeostasis in the subject compared to (e.g., relative to) a control. In some instances, the methods improve glucose homeostasis in the subject compared to the glucose homeostasis in the subject before treatment. In some instances, the methods improve glucose clearance, e.g., from circulation, in the subject compared to a control. In some instances, the methods improve glucose clearance in the subject compared to the glucose clearance in the subject before treatment. Improved glucose clearance may include increased glucose clearance. Increased glucose clearance may reduce blood glucose levels. In some instances, glucose clearance is improved by 1% to 50% including, e.g., by 1% to 40%, by 1% to 30%, by 1% to 20%, or by 1% to 10%.

In some embodiments, the methods reduce adipose tissue mass (e.g., the amount of adipose tissue or fat) in the subject. The term “adipose tissue” refers to fat including, e.g., the connective tissue that stores fat. Adipose tissue contains multiple regenerative cell types, including, e.g., adipose derived stem cells (ASCs) and endothelial progenitor and precursor cells. Types of adipose tissue of interest include, but are not limited to, white adipose tissue and brown adipose tissue. In some instances, the methods reduce adipose tissue mass compared to a control. In some instances, the methods reduce adipose tissue mass in a subject compared to the adipose tissue mass in the subject before treatment. In some instances, the methods reduce adipose tissue by 1% to 50% including, e.g., by 1% to 40%, by 1% to 30%, by 1% to 20%, or by 1% to 10%. In some instances, the methods reduce adipose tissue by 10% to 50% including, e.g., by 10% to 40%, by 10% to 30%, by 10% to 20%, by 20% to 50%, by 30% to 50%, or by 40% to 50%. In some instances, the methods reduce the amount of white fat compared to a control. In some instances, the methods reduce the amount of white fat in a subject compared to the amount of white fat in the subject before treatment. In some instances, the methods reduce white fat by 1% to 50% including, e.g., by 1% to 40%, by 1% to 30%, by 1% to 20%, or by 1% to 10%. In some instances, the methods reduce white fat by 10% to 50% including, e.g., by 10% to 40%, by 10% to 30%, by 10% to 20%, by 20% to 50%, by 30% to 50%, or by 40% to 50%. In some instances, the methods reduce the amount of brown fat compared to a control. In some instances, the methods reduce the amount of brown fat in a subject compared to the amount of brown fat in the subject before treatment. In some instances, the methods reduce brown fat by 1% to 50% including, e.g., by 1% to 40%, by 1% to 30%, by 1% to 20%, or by 1% to 10%. In some instances, the methods reduce brown fat by 10% to 50% including, e.g., by 10% to 40%, by 10% to 30%, by 10% to 20%, by 20% to 50%, by 30% to 50%, or by 40% to 50%. In some instances, the methods reduce the amount of smaller epididymal fat compared to a control. In some instances, the methods reduce the amount of smaller epididymal fat in a subject compared to the amount of smaller epididymal fat in the subject before treatment. In some instances, the methods reduce smaller epididymal fat by 1% to 50% including, e.g., by 1% to 40%, by 1% to 30%, by 1% to 20%, or by 1% to 10%. In some instances, the methods reduce smaller epididymal fat by 10% to 50% including, e.g., by 10% to 40%, by 10% to 30%, or by 10% to 20%. In some instances, the methods reduce the amount of subcutaneous inguinal fat compared to a control. In some instances, the methods reduce the amount of subcutaneous inguinal fat in a subject compared to the amount of subcutaneous inguinal fat in the subject before treatment. In some instances, the methods reduce subcutaneous inguinal fat by 1% to 50% including, e.g., by 1% to 40%, by 1% to 30%, by 1% to 20%, or by 1% to 10%. In some instances, the methods reduce subcutaneous inguinal fat by 10% to 50% including, e.g., by 10% to 40%, by 10% to 30%, by 10% to 20%, by 20% to 50%, by 30% to 50%, or by 40% to 50%.

The “control,” as used herein in its conventional sense, may be any suitable control. In some embodiments, the control includes a subject, e.g., a subject with a metabolic disorder, to whom an effective amount of CES2 active agent has not been administered. The control may be a subject that has the same metabolic disorder(s) and/or associated conditions as the treated subject. In some embodiments, the control includes a subject to whom an effective amount of a CES2 active agent is not administered, where the subject has a metabolic disorder or a combination of metabolic disorders and/or associated conditions that match those of the subject to whom the effective amount is administered. In some instances, the control subject has characteristics (e.g., age, sex, height, weight, race, diet, etc.) that are shared by the treated subject. In some instances, the outcomes for a subject as described herein (related to, e.g., food intake by the subject, body weight of the subject, glucose homeostasis in the subject, adipose tissue mass of the subject, etc.) are measured relative to the subject before treatment.

Embodiments of the methods can be practiced on any suitable subject. A subject of the present invention may be a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to human subjects of both genders and at any stage of development (i.e., neonates, infant, juvenile, adolescent, adult), where in certain embodiments the human subject is a juvenile, adolescent or adult.

### Pharmaceutical Formulations

As summarized above, pharmaceutical formulations or pharmaceutical compositions are provided. A pharmaceutical formulation may include CES2 active agent, and one or more of pharmaceutically acceptable carriers or excipients. In some instances, a pharmaceutical formulation includes an amount of a CES2 active agent effective to treat a metabolic disease; and an excipient. The CES2 active agent may be any suitable CES2 active agent, e.g., as described herein. The amount of CES2 active agent, e.g., effective amount, may be any suitable amount according to any of the embodiments described herein. The pharmaceutical formulations may be administered in combination with any of the therapies (e.g., therapies for treating a metabolic disorder) as described herein.

The pharmaceutical formulation may be formulated for administration by any suitable means. In certain embodiments, the composition is formulated for administration orally, intradermally, intramuscularly, parenterally, intravenously, intra-arterially, intracranially, subcutaneously, intraorbitally, intraventricularly, intraspinally, intraperitoneally, or intranasally. The pharmaceutical formulations or compositions can be formulated into various dosage forms, including tablets, powders, fine granules, granules, dry syrups, capsules, liquid compositions, etc. In some instances, the pharmaceutical formulation is a capsule or tablet. In some instances, the pharmaceutic formulation is a parenteral formulation. In some instances, the pharmaceutical formulation is an intraperitoneal formulation.

Additives and diluents normally utilized in the pharmaceutical arts can optionally be added to the pharmaceutical formulation. These include thickening, granulating, dispersing, flavoring, sweetening, coloring, and stabilizing agents, including pH stabilizers, other excipients, anti-oxidants (e.g., tocopherol, BHA, BHT, TBHQ, tocopherol acetate, ascorbyl palmitate, ascorbic acid propyl gallate, and the like), preservatives (e.g., parabens), and the like. Exemplary preservatives include, but are not limited to, benzylalcohol, ethylalcohol, benzalkonium chloride, phenol, chlorobutanol, and the like. Some useful antioxidants provide oxygen or peroxide inhibiting agents for the formulation and include, but are not limited to, butylated hydroxytoluene, butylhydroxyanisole, propyl gallate, ascorbic acid palmitate, α-tocopherol, and the like. Thickening agents, such as lecithin, hydroxypropylcellulose, aluminum stearate, and the like, may improve the texture of the formulation.

A container for holding the CES2 active agent formulation or CES2 active agent pharmaceutical composition may be configured to hold any suitable volume of the CES2 active agent formulation or composition. In some cases, the size of the container may depend on the volume of CES2 active agent composition to be held in the container. In certain embodiments, the container may be configured to hold an amount of CES2 active agent composition ranging from 0.1 mg to 1000 mg, such as from 0.1 mg to 900 mg, such as from 0.1 mg to 800 mg, such as from 0.1 mg to 700 mg, such as from 0.1 mg to 600 mg, such as from 0.1 mg to 500 mg, such as from 0.1 mg to 400 mg, or 0.1 mg to 300 mg, or 0.1 mg to 200 mg, or 0.1 mg to 100 mg, 0.1 mg to 90 mg, or 0.1 mg to 80 mg, or 0.1 mg to 70 mg, or 0.1 mg to 60 mg, or 0.1 mg to 50 mg, or 0.1 mg to 40 mg, or 0.1 mg to 30 mg, or 0.1 mg to 25 mg, or 0.1 mg to 20 mg, or 0.1 mg to 15 mg, or 0.1 mg to 10 mg, or 0.1 mg to 5 mg, or 0.1 mg to 1 mg, or 0.1 mg to 0.5 mg. In certain embodiments, the container is configured to hold an amount of a CES2 active agent composition ranging from 0.1 g to 10 g, or 0.1 g to 5 g, or 0.1 g to 1 g, or 0.1 g to 0.5 g. In certain instances, the container is configured to hold a volume (e.g., a volume of a liquid CES2 active agent composition) ranging from 0.1 ml to 200 ml. For instance, the container may be configured to hold a volume (e.g., a volume of a liquid) ranging from 0.1 ml to 1000 ml, such as from 0.1 ml to 900 ml, or 0.1 ml to 800 ml, or 0.1 ml to 700 ml, or 0.1 ml to 600 ml, or 0.1 ml to 500 ml, or 0.1 ml to 400 ml, or 0.1 ml to 300 ml, or 0.1 ml to 200 ml, or 0.1 ml to 100 ml, or 0.1 ml to 50 ml, or 0.1 ml to 25 ml, or 0.1 ml to 10 ml, or 0.1 ml to 5 ml, or 0.1 ml to 1 ml, or 0.1 ml to 0.5 ml. In certain instances, the container is configured to hold a volume (e.g., a volume of a liquid CES2 active agent composition) ranging from 0.1 ml to 200 ml.

The shape of the container may also vary. In certain cases, the container may be configured in a shape that is compatible with the assay and/or the method or other devices used to perform the assay. For instance, the container may be configured in a shape of typical laboratory equipment used to perform the assay or in a shape that is compatible with other devices used to perform the assay. In some instances, the container is a liquid container. In some embodiments, the liquid container is a vial or a test tube. In certain cases, the liquid container is a vial. In certain cases, the liquid container is a test tube. In some instances, the container is a blister pack.

As described above, embodiments of the container can be compatible with the CES2 active agent composition. Examples of suitable materials for the containers include, but are not limited to, glass and plastic. For example, the container may be composed of glass, such as, but not limited to, silicate glass, borosilicate glass, sodium borosilicate glass (e.g., PYREX™) fused quartz glass, fused silica glass, and the like. Other examples of suitable materials for the containers include plastics, such as, but not limited to, polypropylene, polymethylpentene, polytetrafluoroethylene (PTFE), perfluoroethers (PFE), fluorinated ethylene propylene (FEP), perfluoroalkoxy alkanes (PFA), polyethylene terephthalate (PET), polyethylene (PE), polyetheretherketone (PEEK), and the like.

In some embodiments, the container may be sealed. That is, the container may include a seal that substantially prevents the contents of the container from exiting the container. The seal of the container may also substantially prevent other substances from entering the container. For example, the seal may be a water-tight seal that substantially prevents liquids from entering or exiting the container, or may be an air-tight seal that substantially prevents gases from entering or exiting the container. In some instances, the seal is a removable or breakable seal, such that the contents of the container may be exposed to the surrounding environment when so desired, e.g., if it is desired to remove a portion of the contents of the container. In some instances, the seal is made of a resilient material to provide a barrier (e.g., a water-tight and/or air-tight seal) for retaining a sample in the container. Particular types of seals include, but are not limited to, films, such as polymer films, caps, etc., depending on the type of container. Suitable materials for the seal include, for example, rubber or polymer seals, such as, but not limited to, silicone rubber, natural rubber, styrene butadiene rubber, ethylene-propylene copolymers, polychloroprene, polyacrylate, polybutadiene, polyurethane, styrene butadiene, and the like, and combinations thereof. For example, in certain embodiments, the seal is a septum pierceable by a needle, syringe, or cannula. The seal may also provide convenient access to a sample in the container, as well as a protective barrier that overlies the opening of the container. In some instances, the seal is a removable seal, such as a threaded or snap-on cap or other suitable sealing element that can be applied to the opening of the container. For instance, a threaded cap can be screwed over the opening before or after a sample has been added to the container.

### Utility

The subject methods and formulations find use in applications, e.g., clinical applications, involving metabolic disorders and one or more conditions associated with metabolic disorders. In some embodiments, the methods and formulations find use in applications where it is desirable to treat a metabolic disorder and one or more conditions associated with metabolic disorders including, e.g., obesity, an obesity related disorder, diabetes, etc. In some instances, the methods and formulations find use in applications where it is desirable to prevent the development or occurrence of a metabolic disorder and one or more conditions associated with metabolic disorders. In certain embodiments, the methods and formulations find use in applications where weight loss for a subject is desirable. In certain embodiments, the methods and formulations find use in applications where improving glucose homeostasis in a subject is desirable. In certain embodiments, the methods and formulations find use in applications where it is desirable to induce a physical activity associated outcome in a subject. In certain embodiments, the methods and formulations find use in applications where it is desirable to treat a nervous system disorder. The methods and formulations may also find use in combination with other therapies and treatments for any of the disorders and associated conditions described herein.

The following example(s) is/are offered by way of illustration and not by way of limitation.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### 1. Organism-Wide Secretome Mapping of Tissue Crosstalk in Exercise

### A. Summary

Exercise is a powerful physiologic stimulus that provides benefits to multiple organ systems and confers protection against disease. These effects are in part mediated by blood-borne factors that mediate tissue crosstalk and function as molecular effectors of physical activity. To globally understand how physical activity reshapes cellular secretomes, here we use a proximity biotinylation approach to profile cell type-specific secretomes following treadmill running in mice. This organism-wide, 21-cell type, 10-tissue secretome atlas reveals complex, bidirectional, and cell type-specific regulation of secreted proteins following exercise training. We identify a gradient of secretome responses across cell types, with secretomes from Pdgfra-cre labeled cells being one of the most exercise-responsive in the entire dataset. Peptide-level correlation analysis uncovers exercise regulation of cell type-specific secreted proteoforms. Finally, we show that exercise-inducible, liver-derived secreted CES2 proteins modulate systemic energy metabolism and suppress obesity in high fat diet-fed mouse models. Together, our studies map exercise-regulated cell types and secreted proteins and illuminate the dynamic remodeling of cell and tissue crosstalk by physical activity.

### B. Introduction

Physical activity is a powerful physiologic stimulus that provides benefits to many organ systems and confers protection against disease (Hawley et al., 2014; Neufer et al., 2015; Piercy et al., 2018; Warburton and Bredin, 2017). Conversely, physical inactivity is a major contributor to cardiovascular morbidity and mortality (Booth et al., 2017; Lear et al., 2017). The magnitude of the benefits of physical activity is comparable, and in some cases even greater, than currently available first line pharmacological treatments (Blair et al., 1989; Hambrecht et al., 2004; Knowler et al., 2002; Myers et al., 2002; Rush et al., 2006). The mechanisms responsible for the benefits of exercise are incompletely understood, but likely extend beyond activity-associated increases in energy expenditure alone (McGee and Hargreaves, 2020; Neufer et al., 2015; Ruegsegger and Booth, 2018).

In recent years, there has been tremendous interest in the identification and characterization of exercise-inducible, soluble (e.g., secreted) blood-borne molecules. These circulating molecules, which have been called “exerkines” or “exercise factors,” are secreted signaling molecules that function as molecular effectors of physical activity (Chow et al., 2022; Safdar et al., 2016; Severinsen and Pedersen, 2020). Over 50 years ago, Goldstein (Goldstein, 1961) demonstrated that contracting muscle from dogs produced a humoral factor that stimulated glucose uptake when transferred to non-exercised muscle preparations. More recently, experiments involving re-infusion of exercise-conditioned plasma in mice have also provided additional evidence for bioactive molecules present in the circulation following exercise (De Miguel et al., 2021; Horowitz et al., 2020).

At a molecular level, many individual candidate metabolites, lipids, polypeptides and proteins have been proposed to function as exerkines (Agudelo et al., 2014; Boström et al., 2012; Chow et al., 2022; Knudsen et al., 2020; Lynes et al., 2017; Meex et al., 2015; Rao et al., 2014; Reddy et al., 2020; Sato et al., 2022; Steensberg et al., 2000; Takahashi et al., 2019; Wang et al., 2020a; Wrann et al., 2013; Yang et al., 2021). However, these previous efforts have typically focused on a single factor (e.g., IL-6) and/or a single cell type/tissue of origin (e.g., muscle). Few studies have systematically mapped exercise-inducible secreted molecules across an entire organism. A major challenge, especially for secreted polypeptides and proteins, has been the low depth of plasma proteome coverage by classical shotgun proteomics techniques (Anderson and Anderson, 2002; Uhlén et al., 2015). Aptamer- and antibody-based approaches provide higher sensitivity, but are not comprehensive for the plasma proteome and cannot be used to detect the array of potentially new proteoforms or cleavage fragments that might be produced following physical activity (Boström et al., 2012; Somineni et al., 2014; Zhang et al., 2022). Finally, many secreted proteins are expressed by multiple cell types, and single snapshot detection of these molecules in the circulation would not be expected to enable detection of cell type-specific, and potentially bidirectional regulation of exercise-inducible changes across distinct cell types.

We (Wei et al., 2021; Wei et al., 2020) and others (Droujinine et al., 2021; Kim et al., 2021; Liu et al., 2021) have recently described a biochemical secretome profiling methodology that enables direct labeling, enrichment, and identification of secreted proteins in mice at a cell type-specific resolution. Key to this methodology is the delivery of an engineered biotinylation enzyme TurbolD (Branon et al., 2018) into the secretory pathway of cells via adeno-associated virus (AAV) transduction. Cell type-specific labeling is achieved genetically because the expression of the TurbolD is restricted to those cells expressing cre recombinase. Biotinylated and secreted plasma proteins can then be purified directly from blood plasma using streptavidin beads and analyzed by LC-MS/MS. Our initial secretome studies with the proximity biotinylation approach previously established that cell type-specific secretome changes could be profiled directly in an intact animal (Wei et al., 2020). Here, we have applied secretome profiling at organism-wide scale to examine the cell type-specific secretome response to exercise training. Our organism-wide 21-cell type, 10-tissue secretome map of physical activity provides fundamental insights into the molecular identity and cellular origin of exercise-regulated circulating factors and illuminates the dynamic regulation of intercellular and inter-organ crosstalk by physical activity.

### C. Materials and Methods

### 1. Experimental Models and Subject Details

a. Mouse models. Animal experiments were performed according to procedures approved by the Stanford University IACUC. Mice were maintained in 12 h light-dark cycles at 22° C. and ˜50% relative humidity and fed a standard irradiated rodent chow diet. Where indicated, high fat diet (Research Diets, D12492) was used. C57BL/6J male and female mice (stock no. 000664), homozygous Alb-cre male mice (stock no. 003574), hemizygous Cdh16-cre male mice (stock no. 012237), homozygous Gcg-icre mice male mice (stock no. 030663), hemizygous Pdx1-cre male mice (stock no. 014647), homozygous Myh6-creER male mice (stock no. 005657), hemizygous MCK-cre male mice (stock no. 006475), hemizygous Myh11-icreER male mice (stock no. 019079), homozygous Cdh5-cre male mice (stock no. 006137), heterozygous Pdgfra-creER male mice (stock no. 032770), homozygous Pdgfrb-creER male mice (stock no. 030201), hemizygous Vil1-cre male mice (stock no. 021504), homozygous Sftpc-creER male mice (stock no. 028054), hemizygous Col1a1-creER male mice (stock no. 016241), hemizygous CD2-cre male mice (stock no. 008520), hemizygous Lck-cre male mice (stock no. 003802), hemizygous Nr5a1-creER male mice (stock no. 033687), hemizygous Nes-creER male mice (stock no. 016261), hemizygous Syn1-cre male mice (stock no. 003966), hemizygous Adipoq-cre male mice (stock no. 028020), hemizygous Ucp1-cre male mice (stock no. 024670), and hemizygous LysM-cre male mice (stock no. 031674) were purchased from Jackson Laboratory. All the cre driver male mice were crossed with female C57BL/6J mice to generate hemizygous/heterozygous mice. Genotypes were verified following genotyping protocols and using the primers listed on the Jackson Laboratory website.

### 2. Methods Details

a. Cell line cultures. HEK293T cells were obtained from ATCC (CRL-3216) and cultured in complete medium (Dulbecco's Modified Eagle's Medium, Corning, 10013CV; 10% FBS, Corning, 35010CV; 1:1,000 penicillin-streptomycin, Gibco, 15140-122). Cells were grown at 37° C. with 5% CO₂. For transient transfection, cells were transfected in 10 cm² at ˜60% confluency using PolyFect (Qiagen, 301107) and washed with complete medium 6 h later.
b. Western blotting. For analyzing samples using Western blot, proteins were separated on NuPAGE 4-12% Bis-Tris gels and transferred to nitrocellulose membranes. Equal loading was ensured by staining blots with Ponceau S solution. Blots were then incubated with Odyssey blocking buffer for 30 min at room temperature and incubated with primary antibodies (1:1000 dilution mouse anti-V5 antibody (Invitrogen, R960-25), 1:1000 dilution mouse anti-FLAG antibody (Sigma, F1804), 1:5000 dilution rabbit anti-β-tubulin antibody (Abcam, ab6046), 1:1000 dilution rabbit anti-CES2 antibody (Novus Biologicals, NBP1-91620), 1:500 rabbit anti-TIMP3 antibody (Thermo Fisher, 710404), 1:500 rabbit anti-F13A antibody (MyBioSource, MBS2026456), 1:500 rabbit anti-ITIH2 (MyBioSource, MBS9612213), 1:500 dilution rabbit anti-C4BPA (Abnova, H00000722-D01P), 1:5000 dilution goat anti-albumin antibody (Novus biological, NB600-41532), 1:1000 dilution rabbit anti-H6PD antibody (Abcam, ab170895), 1:1000 dilution rabbit anti-BHMT (Abcam, ab96415), 1:1000 dilution streptavidin Alexa Fluor 680 (Thermo Fisher, S32358)) in blocking buffer overnight at 4° C. Blots were washed three times with PBST (0.05% Tween-20 in PBS) and stained with species-matched secondary antibodies (1:10000 dilution goat anti-mouse IRDye 680RD (LI-COR, 925-68070), 1:10000 dilution goat anti-rabbit IRDye 800RD (LI-COR, 925-68070), 1:10000 dilution donkey anti-goat IRDye 800CW (LI-COR, 925-32214)) at room temperature for 1 h. Blots were further washed three times with PBST and imaged with the Odyssey CLx Imaging System. Secondary antibodies were not required for imaging blots incubated with streptavidin Alexa Fluor 680 primary antibody.
c. AAV production. pAAV-FLEx-ER-TurbolD (Addgene, 160857), pAAV-Tbg-CES2A-ΔC, pAAV-Tbg-CES2C-ΔC plasmids were amplified, extracted using an endotoxin-free Qiagen Maxiprep kit (Qiagen, 12362) and sequence verified. AAV9-FLEx-ER-TurbolD (60221S), AAV8-Tbg-CES2A-ΔC (63849S), AAV8-Tbg-CES2C-ΔC (63850S) viruses were made with Penn Vector Core.
d. Viral transduction. For transduction of brain-specific cre driver lines (Syn1-cre and Nes-creER mice), injection was carried out as previously reported (Gombash Lampe et al., 2014). Briefly, postnatal day 1 pups were anesthetized on ice for 30-60 s. The temporal vein was identified under a dissection microscope and injected with 10e11 genome copies (GC) of AAV9-FLEx-ER-TurbolD virus per mouse diluted in a total volume of 30 μl saline (containing 0.3 μl 0.4% Trypan blue solution) with a 31G syringe (BD, 328290). Injected pups were then recovered in hands for 30 s and returned to home cages. For Syn1-cre mice, 8 to 9 weeks after injection, 1-week treadmill running was performed as described below on male mice with the correct genotype. For Nes-creER mice, 5 to 6 weeks after injection. Tamoxifen (Sigma, T5648-1G) was prepared as a 20 mg/ml solution in corn oil and administered daily for 5 d (100 μl per day, intraperitoneally) to induce recombination. 3 weeks after the final tamoxifen injection, 1-week treadmill running was performed on male mice with the correct genotype. For transduction of cre/icre driver mice (except Syn1-cre mice), 6-week-old male hemizygous mice were injected via tail vein with a 29G syringe (Thermo Fisher, 14-841-32) at a dose of 3*10e11 GC per mouse diluted in saline in a total volume of 100 μl per mouse. Three weeks after transduction with the AAV9-FLEx-ER-TurbolD virus, 1-week treadmill running was performed. For transduction of creER/icreER mice (except Nes-creER mice), AAV9-FLEx-ER-TurbolD virus was injected via tail vein into 6-week-old hemizygous/heterozygous male mice at a dose of 3*10e11 GC per mouse. After a 2-week transduction period, tamoxifen (Sigma, T5648-1G) was prepared as a 20 mg/ml solution in corn oil and administered daily for 5 d (100 μl per day, intraperitoneally) to induce recombination. After the final tamoxifen injection, mice were housed in their home cages for 3 additional weeks before performing 1-week treadmill running. For transduction of C57BL/6J mice, AAV8-Tbg-CES2A-ΔC/CES2C-ΔC/GFP viruses were injected via tail vein into 8 to 10-week-old male mice at a dose of 10e11 GC per mouse. One week after viral transduction, mice were fed with HFD (60% fat, Research Diets, D12492). Body weights and food intake were measured every week. After 6 weeks of HFD feeding, glucose tolerance and insulin tolerance tests were performed. At the end of 7 weeks of HFD feeding, tissues and blood were collected for further analysis.
e. Mouse exercise and secretome labeling protocols. A 6-lane animal treadmill (Columbus Instruments, 1055-SRM-D65) was used for mouse running. Prior to treadmill running, the bodyweight of individual mice was measured with a tabletop scale. Mice were then acclimated to the treadmill for 5 minutes before running at a speed of 5 m/min for 5 min. Then the speed was increased to 20 m/min and kept constant for 60 min. A serological pipette was used to manually stir the mice to avoid excessive electrical shock during the whole running period. After exercise, mice were returned to home cages. Running was performed for 7 consecutive days in the morning (between 9-12 am). On the fourth day of one-week treadmill running, biotin water (0.5 mg/ml) was supplemented to initiate labeling and kept accessible to mice until the end of the experiment. On the next day, an additional dose of biotin was administered by injection (24 mg/ml, intraperitoneally, in a solution of 18:1:1 saline:Kolliphor EL:DMSO, final volume of 200 μl per mouse per day) 1 h prior to the running for 3 consecutive days.
f. Quantitative PCR. We collected the following tissues from the indicated genotypes: liver from Alb-cre mice, heart from Myh6-creER and Myh11-icreER mice, brain from Syn1-cre and Nes-creER mice, iWAT from Adipoq-cre mice, BAT from Ucp1-cre mice, lung from Sftpc-creER, Pdgfra-creER, Pdgfrb-creER and Cdh5-cre mice, quadricep muscle from MCK-cre mice, intestines from Vil1-cre mice, pancreas from Gcg-icre and Pdx1-cre mice, kidney from Cdh16-cre mice, adrenal gland from Nr5a1-cre mice, hind limb with muscles removed from Col1a1-creER mice. For Lck-cre and CD2-icre mice, splenocytes were collected by passing the spleen through a cell strainer (Corning, 352350) and resuspended in 2% BSA solution. Splenocytes were stained with FITC anti-mouse TCRβ(BioLegend, 109206), Percp/Cy5.5 anti-mouse CD19 (BioLegend, 152406) and LIVE/DEAD Aqua (Invitrogen, L34957). αβT cells from Lck-cre mice were gated on Aqua-CD19-TCRβ+, isolated with FACS, and spun down at 300 g for 5 min at 4° C. for downstream analysis. For CD2-icre mice, αβT cells were gated on Aqua-CD19-TCRβ+ and B cells were gated on Aqua-CD19+TCRβ−. Both αβT cells and B cells were sorted with FACS, mixed, and spun down at 300 g for 5 min at 4° C. for downstream analysis. For Lysm-creER mice, a new cohort of 6 to 8-week old mice (N=3) was transduced with AAV9-FLEx-ER-TurbolD virus and was injected with tamoxifen to induce cre recombination the same as described before. 3 weeks after the final tamoxifen injection, 1 ml 3% (w/v) thioglycolate solution (Thermo Fisher, B111716) was intraperitoneally injected. 5 days later, 10 ml ice-cold DPBS (Thermo Fisher, 14190144) was intraperitoneally injected to isolate the accumulated macrophages in the peritoneal cavity. Cells were spun down at 400 g for 10 min at 4° C. for downstream analysis. Isolated cells or 30-50 mg of frozen tissues were added to bulk tubes (Thermo Fisher, 15340162) containing metal beads and 1 ml TRIzol Reagent (Invitrogen, 15596026). Tissues were then homogenized using a Benchmark BeadBlaster Homogenizer at 4° C. The mixture was spun down at 13,000 rpm for 10 min at 4° C. to pellet the insoluble materials. RNA was extracted using a RNeasy Mini Kit (Qiagen, 74106) and reverse-transcribed using a High-Capacity cDNA Reverse Transcription Kit (Thermo Fisher, 4368813). Quantitative PCR was performed using Ssoadvance Universal SYBR Green mix (Biorad, 1725272) with a CFX Opus Real-Time PCR instrument. All values were normalized by the ΔΔCt method to Rps18.
g. Plasma and tissue sample preparation from mice. 2 h after the final bout of running, blood was collected via submandibular bleeding using a 21G needle (BD, 305129) into lithium heparin tubes (BD, 365985) and immediately spun down at 5,000 rpm for 5 min at 4° C. to retrieve the plasma fractions. All tissues were dissected, weighed on a scale, collected into Eppendorf tubes, and immediately frozen on dry ice and stored at −80° C. Adipose tissues were collected into 4% paraformaldehyde for histology analysis. For western blot analysis, tissues were mixed with 0.5 ml of cold RIPA buffer and homogenized using a Benchmark BeadBlaster Homogenizer at 4° C. The mixture was spun down at 13,000 rpm for 10 min at 4° C. to pellet the insoluble materials. The supernatant was quantified using a tabletop Nanodrop One and analyzed by western blot. To remove remaining biotin from blood plasma, 200 μl plasma from a single mouse was added with 15 ml PBS and subsequently concentrated 30-fold using 3 kDa filter tubes (Millipore, UFC900324) by spinning down at 4,000 rpm for 1 h. The flowthrough was discarded, and the dilution and centrifugation steps were repeated until a final solution of 500 μl was retrieved at a 9000-fold final dilution. To enrich biotinylated plasma proteins, 200 μl Dynabeads MyOne Streptavidin T1 magnetic beads (Thermo Fisher, 65602) were washed twice with 1 ml washing buffer (50 mM Tris-HCl, 150 mM NaCl, 0.1% SDS, 0.5% sodium deoxycholate, 1% NP-40, 1 mM EDTA, 1×HALT protease inhibitor, 5 mM trolox, 10 mM sodium azide and 10 mM sodium ascorbate) and resuspended in 100 μl washing buffer. The beads were then added to the 500 μl biotin-free plasma solution and incubated at 4° C. overnight with rotation. The beads were subsequently washed twice with 1 ml washing buffer, once with 1 ml 1 M KCl solution, once with 1 ml 0.1 M Na₂CO₃ solution, once with 1 ml 2 M urea in 10 mM Tris-HCl (pH 8.0), and twice with 1 ml washing buffer. Eppendorf tubes containing beads were vortexed for 3s between each step to ensure thorough washing. Finally, biotinylated proteins were eluted by boiling at 95° C. for 10 min in 60 μl of 2× sample buffer supplemented with 20 mM DTT and 2 mM biotin. Successful enrichment of biotinylated plasma proteins was validated by running the elution sample on NuPAGE 4-12% Bis-Tris gels followed by silver staining (Thermo Fisher, LC6070) according to the instructions from the manufacturer's protocol.
h. Proteomic sample processing. After cooling down to room temperature for 3 min, boiled streptavidin-purified plasma samples (60 μl) were digested using a Mini S-Trap protocol provided by the manufacturer (Protifi, C02-micro-80). As previously described (Wei et al., 2021), cysteine residues were first alkylated by incubating in 30 mM iodoacetamide (Sigma, A3221) in the dark at room temperature for 30 min. Samples were then acidified with phosphoric acid at a final concentration of 1.2%. 420 μl bind/wash buffer (100 mM tetraethylammonium bromide (TEAB) in 90% methanol) was added to each sample. 150 μl samples were loaded onto micro S-trap columns and spun down at 4000 g for 20 s. The flow-through was discarded, and the centrifugation step was repeated until all the solution passed through the column. Following four washes with 150 ul bind/wash buffer, 1 μg trypsin (Promega, V5113) was added to the S-trap and incubated at 47° C. for 90 min. After trypsinization, peptides were washed once with 50 mM TEAB (40 μl), once 0.2% formic acid (40 μl), once with a mixture of 50% acetonitrile and 0.2% formic acid (40 μl) and once of 0.2% formic acid in water (40 μl) by spinning down at 1,000g for 60 s. Eluted fraction from each wash was combined, lyophilized, resuspended in 0.2% formic acid, normalized to concentration using a Nanodrop Spectrophotomerter (Thermo Fisher, absorbance at 205 nm), and analyzed by LC-MS/MS. One microliter of each sample was taken and combined into a pooled sampled that was used to make the chromatogram library.
i. Proteomics data acquisition. Proteomics data were acquired using a spectrum-library free DIA approach that relies on gas-phase fractionation (GPF) to generate DIA-only chromatogram libraries (Pino et al., 2020a; Searle et al., 2018). Peptides were separated over a 25 cm Aurora Series Gen2 reverse-phase LC column (75 μm inner diameter packed with 1.6 μm FSC C18 particles, Ion Opticks). The mobile phases (A: water with 0.2% formic acid and B: acetonitrile with 0.2% formic acid) were driven and controlled by a Dionex Ultimate 3000 RPLC nano system (Thermo Fisher). An integrated loading pump was used to load peptides onto a trap column (Acclaim PepMap 100 C18, 5 μm particles, 20 mm length, Thermo Fisher) at 5 μl/minute, which was put in line with the analytical column 5.5 minutes into the gradient. The gradient was held at 0% B for the first 6 minutes of the analysis, followed by an increase from 0% to 5% B from 6 to 6.5 minutes, and increase from 5 to 22% B from 6.5 to 66.5 minutes, an increase from 22% to 90% from 66.5 to 71 minutes, isocratic flow at 90% B from 71 to 75 minutes, and re-equilibration at 0% B for 15 minutes for a total analysis time of 90 minutes per acquisition. Eluted peptides were analyzed on an Orbitrap Fusion Tribrid MS system (Thermo Fisher). Precursors were ionized was ionized with a spray voltage held at +2.2 kV relative to ground, the RF lens was set to 60%, and the inlet capillary temperature was held at 275° C.

Six chromatogram library files were collected through six repeated injections of the pooled sample only. Here, the instrument was configured to acquire 4 m/z precursor isolation window DIA spectra using a staggered isolation window pattern (Amodei et al., 2019) from narrow mass ranges using window placements optimized by Skyline. DIA MS/MS spectra were acquired with an AGC target of 400,000 charges, a maximum injection time of 54 ms, beam-type collisional dissociation (i.e., HCD) with a normalized collision energy of 33, and a resolution of 30,000 at 200 m/z using the Orbitrap as a mass analyzer. The six gas-phase fractionation chromatogram libraries were collected with nominal mass ranges of 400-500 m/z, 500-600 m/z, 600-700 m/z, 700-800 m/z, 800-900 m/z, and 900-1000 m/z. The exact windowing scheme was downloaded from https://bitbucket(dot)org/searleb/encyclopedia/wiki/Home (Pino et al., 2020a) and is available in Supplementary Information here. Precursor MS1 spectra were interspersed every 25 scans with an AGC target of 400,000 charges, a maximum injection time of 55 ms, a resolution of 60,000 at 200 m/z using the Orbitrap as a mass analyzer, and a scan range of either 395-505 m/z, 495-605 m/z, 595-705 m/z, 695-805 m/z, 795-905 m/z, or 895-1005 m/z.

For quantitative samples (i.e., the non-pooled samples) the instrument was configured to acquire 25×16 m/z precursor isolation window DIA spectra covering 385-1015 m/z using a staggered isolation window pattern with window placements optimized by Skyline (windowing scheme downloaded from the same link as above and available as Supplementary Information here). DIA spectra were acquired with the same MS/MS settings described above. Precursor MS1 spectra were interspersed every 38 scans with a scan range of 385-1015 m/z, an AGC target of 400,000 charges, a maximum injection time of 55 ms, and a resolution of 60,000 at 200 m/z using the Orbitrap as a mass analyzer. The mass spectrometry proteomics data have been deposited to the ProteomeXchange Consortium via the PRIDE partner repository with the dataset identifier PXD034535 (Perez-Riverol et al., 2022).

j. Proteomics data analysis to generate cell type-protein pairs. Staggered DIA spectra were demultiplexed from raw data into mzML files with 10 ppm accuracy using MSConvert (Adusumilli and Mallick, 2017) with settings described in Pino et al (Pino et al., 2020a). Encyclopedia (version 1.12.31) (Searle et al., 2018) was used to search demultiplexed mzML files using an internal PECAN fasta search engine called Walnut (Ting et al., 2017) and a reviewed-plus-isoforms mouse proteome database downloaded Feb. 25, 2022 from Uniprot (Consortium, 2021). Walnut settings were: fixed cysteine carbamidomethylation, full tryptic digestion with up to 2 missed cleavages, HCD (y-only) fragmentation, 10 ppm precursor and fragment mass tolerances, and 5 quantitative ions. The chromatogram library resulting from the Walnut search was then used for Encyclopedia searching, where all similar settings to the Walnut search remained the same, and other settings included a library mass tolerance of 10 ppm, inclusion of both b- and y-type fragment ions, and a minimum number of quantitative ions set at 3. Percolator (version 3.1) was used to filter peptides to a 1% false discovery rate using the target/decoy approach and proteins to a 1% protein-level FDR assuming protein grouping parsimony. Resulting data from EncyclopeDIA were checked in Skyline (Pino et al., 2020b) before further processing with Perseus (Tyanova et al., 2016). Proteins were filtered so that only proteins with 2 or more peptides and those that were detected in all three replicates of at least one condition were retained. Data was converted to cell type-protein pairs, and the median value of the cell type-protein pair intensity was compared to the intensity of that protein detected in WT mice control samples. Keratins were manually removed from our dataset as these proteins are frequently detectable contaminants in mass spectrometry experiments (Mellacheruvu et al., 2013). To remove background labeling contaminants, only cell type-protein pairs that showed a greater than 1.5-fold intensity above the median intensity detected in WT samples were retained (Branon et al., 2018). Then cell type-protein pairs with detected protein intensity across all 6 samples (sedentary and exercise) were included for downstream analysis. Next, cell type-protein pairs with variance >2 ((log 2(maximum intensity)−log 2(minimum intensity))>2 under either sedentary or exercise conditions) were excluded from further analysis (Bourgon et al., 2010). In total, 1272 cell type-protein pairs passed the above filtering criteria and were considered as bona fide cell type-protein pairs.
k. Exercise responsiveness scores calculations. Exercise-regulated cell type-protein pairs (adjusted β-values <0.05) were used for calculating exercise responsiveness scores. Each cell type-protein pair's exercise responsiveness was calculated as abs(log₁₀(adjusted β-values))×abs(log₂(exercise fold change)). Exercise fold change of a cell type-protein pair was defined as median protein abundance across three exercise samples divided by median protein abundance across three sedentary samples of the same genotype. Then the exercise responsiveness scores were calculated by the summarization of the exercise responsiveness of each cell type-protein pair from the same cell type.
l. Time-course of Pdgfra-creER secretomes. AAV9-FLEx-ER-TurbolD virus was injected as previously described into 6-week-old hemizygous male Pdgfra-creER mice at a dose of 3*10e11 GC per mouse. After a 2-week transduction period, tamoxifen was delivered to induce cre-mediated expression of ER-TurbolD. Three weeks after the final tamoxifen injection, mice were divided into three groups (1-day running, 7-day running and sedentary controls, N=3/condition). 1 h before running, biotin was administered by injection (24 mg/ml, intraperitoneally, in a solution of 18:1:1 saline:Kolliphor EL:DMSO, final volume of 400 μl per mouse per day). The treadmill running was carried out as previously described. For the 1-day running group, mice were sacrificed, and blood and tissue samples were analyzed 2 h after a single bout of running. For 7-day running group, mice were run for 7 consecutive days and blood and tissues were harvested 2 h after the final bout of running. For the sedentary group, biotin was administered, and blood and tissue samples were collected 4 h after biotin delivery.
m. Peptide Correlation Analysis. Peptide Correlation Analyses were performed using PeCorA as previously described (Dermit et al., 2021). Briefly, all peptide intensities were uploaded into PeCorA (https://github(dot)com/jessegmeyerlab/PeCorA) for processing. The threshold_to_filter in PeCorA_preprocessing was set to 100. Then adjusted β-values reported by PeCorA were used for downstream analysis and peptides with adjusted β-values <0.05 were considered as “uncorrelated peptides”. Peptide positions within the canonical protein sequence were determined by indexOf function with the sequence information from UniProt (Consortium, 2021) (https://github(dot)com/leolove2022/PeCorAAnalysis(dot)git). Uncorrelated peptides were examined manually, proteins were checked for known cell type-specific expression using BioGPS (Wu et al., 2016), and sequences of discordant peptides were checked for possible known sites of post-translational modification in mammalian datasets using PhosphoSitePlus (Hornbeck et al., 2015) and Uniprot. Boxplots of any “uncorrelated” peptide's abundance compared to the other peptides of the same protein under sedentary and exercise conditions were generated.
n. Gene ontology analysis. Proteins with P_{adj}<0.05 from Pdgfra secretomes were uploaded to online gene ontology analysis tool http://geneontology(dot)org/(Ashburner et al., 2000). The enriched biological processes were ranked by gene ratio and β-values.
o. Isolation and culture of primary mouse hepatocytes. Primary mouse hepatocytes were isolated and cultured as previously described (Jiang et al., 2021; Wei et al., 2020). Briefly, 8 to 12-week-old male mice (C57BL/6J) were sacrificed and perfused with perfusion buffer (1 g/L glucose, 2.1 g/L sodium bicarbonate, 0.4 g/L potassium chloride and 0.2 g/L EDTA in HBSS buffer) via cannulate vena cava for 5 to 8 min and then with digestion buffer (1 mg/ml collagenase IV (Sigma, C5138-1 G) in DMEM/F-12 medium) for 5 to 8 min. The liver was then dissected out, cut into small pieces using a razor blade and passed through a 70-μm cell strainer (BD, 352350) to obtain crude hepatocytes. Cells were then spun down at 50 g for 3 min, resuspended in 10 ml plating medium (10% FBS, 1 μM dexamethasone (Sigma, D4902-100MG), 0.1 μM insulin (Sigma, 91077C), 2 mM sodium pyruvate, 1% penicillin-streptomycin in William's E medium (Quality Biological, 10128-636)) and spun down again at 50 g for additional 3 min. The pellet was resuspended in 10 ml of a 45% Percoll solution in PBS and spun down at 100 g for 10 min to isolate hepatocytes. The final hepatocyte pellet was resuspended in 10 ml plating medium, spun down again at 50 g for 5 min and resuspended in 1 ml plating medium. Cells were counted and plated in a collagen-coated six-well plate at 2 million cells per well. 4 h later, the plating medium was changed to warm maintenance medium (0.1 μM dexamethasone, 1 nM insulin, 0.2% BSA (Sigma, A7906-500G), 2 mM sodium pyruvate, 1% penicillin-streptomycin), and cells were incubated overnight before further treatment.
p. Treatment of hepatocytes with organic compounds, MCT inhibitor and Brefeldin A. 24 h after plating, primary hepatocytes were washed twice with warm PBS to remove BSA. Then 2 ml William's E medium containing the indicated concentrations of sodium lactate (Sigma, 05508-5ML), 2 mM sodium fumarate dibasic (Sigma, F1506-25G), 2 mM sodium succinate dibasic hexahydrate (Sigma, S2378-100G), 2 mM sodium (R)-3-hydroxybutyrate (Sigma, 298360-1G), 2 mM kynurenic acid (Sigma, K3375-250MG), 2 mM D-Pantothenic acid hemicalcium salt (Sigma, 21210-5G-F), 2 mM sodium pyruvate (Sigma, P2256-25G), 2 mM L-(−)-Malic acid (Sigma, 02288-10G) was added. The above organic compounds powder was dissolved in ethyl alcohol 200 proof to make 100 mM master stock and diluted accordingly to reach the indicated concentration in medium. 40 μl ethanol was added as negative control. For MCT inhibitor AR-C155858 (Tocris, 4960) and Brefeldin A (Sigma, B6542-5MG), compound power was dissolved in DMSO to make master stock (100 μM for AR-C155858 and 5 mg/ml for Brefeldin A) and diluted accordingly to reach the indicated concentration in medium containing 2 mM sodium lactate. 4 h later, cells and conditioned medium were harvested and analyzed by western blotting as previously described. For HEK293T cells, cells were washed twice with warm PBS 24 h after transfection and incubated with serum-free medium containing indicated concentration of sodium lactate. 4 h later, cells and conditioned medium were harvested and analyzed by western blotting as described below.
q. Construction of plasmids for overexpression of CES2A/C-ΔC. Flag-CES2A-ΔC fragment (ref sequence NM_133960.5) and Flag-CES2C-ΔC fragment (ref sequence NM_145603.2) were synthesized as gBlocks with IDT. For both fragments, 5′-GACTACAAGGATGACGACGATAAGGGGGGCGGT-3′ (SEQ ID NO:22) sequences (encoding Flag tag) were inserted after CES2 sequences encoding secretory signal peptide (1-78 nt). For Flag-CES2A-ΔC fragment, C-terminal 5′-CATGCAGAGCTG-3′ (SEQ ID NO:23) sequences (encoding HAEL as ER retention signal peptide) were deleted. For Flag-CES2C-ΔC fragment, C-terminal 5′-CACAGGGAGCTT-3′ (SEQ ID NO:24) sequences (encoding HREL as ER retention signal peptide) were deleted. Both gene fragments were inserted into D-TOPO vector using pENTR/D-TOPO Cloning Kit (Invitrogen, K240020) and shuttled into pDEST40 mammalian expression vector using Gateway LR Clonase Enzyme mix (Invitrogen, 11791019). The pDEST40 plasmids were then transformed into One Shot TOP10 Chemically Competent E. coli (Invitrogen, C404010), extracted and sequence verified. pAAV-Tbg-ER-TurbolD plasmid (Addgene, 149415) was cut with restriction enzymes NotI and HindIII to generate the backbone vector. Flag-CES2A-ΔC fragment was amplified using primer sets: 5′-TGCCTTTCTCTCCACAGGTGTCCAGGCGGCCGCGCCACCATGCCATTGGCT AGACTTC-3′ (SEQ ID NO:25), 5′-CCAGAGGTTGATTGGATCCAAGCTTCTACTTGTCCTGAGAACCCTTGAGCTCCTG-3′ (SEQ ID NO:26). Flag-CES2C-ΔC fragment was amplified using primer sets: 5′-TGCCTTTCTCTCCAC AGGTGTCCAGGCGGCCGCGCCACCATGACACGGAACCAACTACATAAC-3′ (SEQ ID NO:27); 5′-CCAGAGGT TGATTGGATCCAAGCTTCTACTTGTCCTGAGAAGCCTTTAGCTCCTGG-3′ (SEQ ID NO:28). Both PCR products were purified using QIAquick gel extraction kit (Qiagen, 28704) and ligated with the linearized pAAV-Tbg vector using Gibson Assembly Master Mix (NEB Biolabs, E2611 L). Ligated plasmids were transformed into One Shot TOP10 Chemically Competent E. coli (Invitrogen, C404010), extracted and sequence verified.
r. Generation of recombinant CES2 proteins. Recombinant CES2A and CES2C proteins were generated by transient transfection of pDEST40-CES2A/C-ΔC plasmids in mammalian Expi293 cells following the manufacturer's instructions. Five to seven days after transfection, conditioned medium was collected, and recombinant proteins were purified using a His GraviTrap TALON column and buffer exchanged to PBS. Protein purity and integrity were analyzed by SDS page. Following purification, recombinant proteins were aliquoted and stored at −80° C. to avoid freeze-thaws.
s. Determination of CES2A-ΔC and CES2C-ΔC secretion in cell culture. HEK293T cells were transfected as described above. 30 h after transfection, cells were washed twice with PBS and added with 10 ml serum-free medium. 12 h later, conditioned medium (10 ml) was collected and concentrated 20-fold using 10 kDa filter tubes (Millipore, UFC801024) to 500 μl. Concentrated conditioned medium was mixed with 4× loading buffer (NuPAGE LDS Sample Buffer, Invitrogen, NP0008, 100 mM DTT) and boiled for 10 min at 95° C. Cells were collected and lysed by probe sonication in RIPA buffer (1% NP-40, 0.1% SDS, 0.5% sodium deoxycholate and 1:100 HALT protease inhibitor, Thermo Fisher, 78429) for 1 min. Cell lysates were spun down at 13000 rpm for 10 min at 4° C. The supernatant was collected, quantified using a tabletop Nanodrop One, boiled for 10 min at 95° C. Both conditioned medium and cell lysate samples were then analyzed by western blot.
t. Histology. Adipose tissues were collected into 4% paraformaldehyde and fixed at 4° C. with rotation for 72 h. Fixing solution was replaced with 20% sucrose solution. Adipose tissues were dehydrated for additional 24 h before freezing in OCT-embedded block (Thermo Fisher, 23-730-571) and cryosectioned. H&E staining was conducted on the slides by Stanford Animal Histology Core.
u. Glucose tolerance and insulin tolerance tests in mice. For glucose tolerance tests, mice were fasted for 6 h (fasting starting 8 am in the morning) and then intraperitoneally injected with glucose at 2 g/kg body weight. Blood glucose levels were measured at 0, 20, 40, 60, and 120 mins via tail bleeding using a glucose meter. For insulin tolerance tests, mice were fasted for 6 h (fasting starting 8 am in the morning) and then intraperitoneally injected with insulin in saline 0.75 U/kg body weight. Blood glucose levels were measured at 0, 20, 40, 60, and 120 mins via tail bleeding using a glucose meter.
v. Carboxylesterases enzymatic activity measurement. A continuous spectrophotometric assay was performed using 4-nitrophenyl acetate (Sigma, N8130-5G) as substrate as previously described (Ross and Borazjani, 2007). Briefly, 1 mM 4-nitrophenyl acetate was prepared freshly in 50 mM Tris-CI buffer (pH 7.4). 150 μl of 4-nitrophenyl acetate solution was added into a single well of a 96-well plate (Thermo Fisher, 125565501), followed by pre-incubating 5 min at 37° C. in the absorbance plate reader. 15 μg liver lysates or 3 μl plasma were diluted in 50 mM Tris·Cl buffer (pH 7.4) and added to 4-nitrophenyl acetate solution (total volume 300 μl). The formation of β-nitrophenolate was measured every 30 s at 405 nm for 5 min. Subtracted from background absorbance, absorbance at each time point was used to generate a kinetic plot for each sample. Data points within the linear range of the reaction were used to calculate the slope of the enzymatic reaction. Finally, relative enzymatic activity was calculated comparing the slopes of reaction from each sample.

### 3. Quantification and Statistical Analysis

a. Data representation and statistical analysis. All values in figures are shown as mean±SEM. The number of biological replicates (N) is described in each figure legend (N corresponds to the number of animals used under each condition for animal experiments and corresponds to the number of independently conducted experiments for cellular experiments). For animal studies, mice were randomly assigned to control and treatment groups. Each animal study was repeated at least twice using separate cohorts of mice. To identify statistically changed cell type-protein pairs (exercise vs sedentary, N=3/protein/condition/genotype), protein intensities were first scaled by the scale( ) function in R package (Alvarez-Castelao et al., 2017). The Limma package (Law et al., 2016; Ritchie et al., 2015) was implemented to conduct the moderated t-statistics (Smyth, 2004) (https://github(dot)com/leolove2022/ModeratedtTest(dot)git), and adjusted β-values of each cell type-protein pair were generated into excel files. 256 cell type-protein pairs out of total 1272 pairs were defined as under exercise regulation (adjusted β-values <0.05). Each in vitro experiment using primary cells from mice was repeated using at least three cohorts of mice. Two-tailed, unpaired student's t-test was used for single comparisons assuming the sample groups exhibited a normal distribution and comparable variance. Two-way ANOVA with post hoc Sidak's multiple comparisons test with repeated measures was used for the body weights, glucose tolerance test and insulin tolerance test studies. Unless otherwise specified, statistical significance was set at adjusted β-value <0.05 for the proteomics data, and β-value <0.05 for all other comparisons.

### D. Results

### 1. Study Design and Proteomic Map of Exercise-Regulated Secretomes

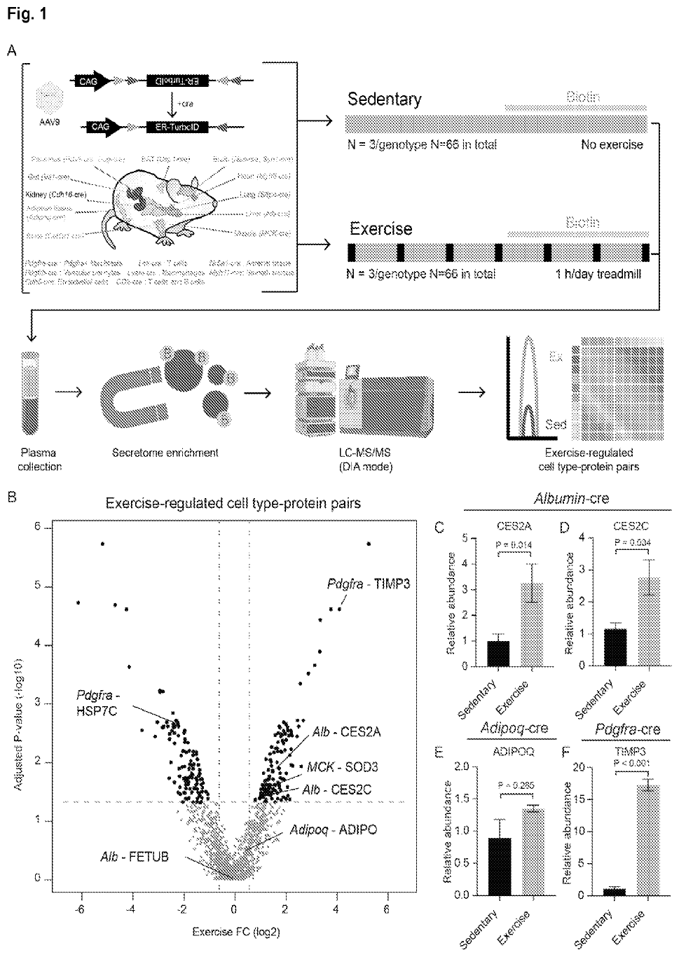

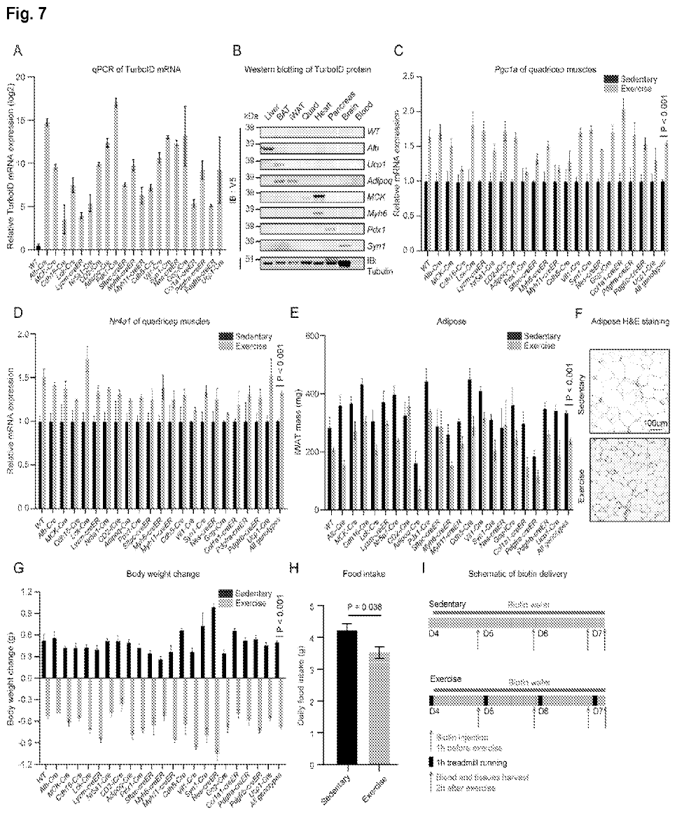

The experimental design for organism-wide, cell type-specific secretome profiling in response to exercise is shown schematically in FIG. 1A. First, we generated a cohort of 21 manually-curated hemizygous cre mouse driver lines from the Jackson Laboratories (N=6/genotype, see Methods). Included amongst the set of cre driver lines were those that target specific organs previously shown to participate in response to exercise (e.g., MCK-cre targeting muscle, Adiponectin-cre targeting fat, and Albumin-cre targeting liver). Other cre drivers exhibited well-validated expression patterns (e.g., Lysm-cre for macrophages), even though those cell types had not been previously implicated in the response to physical activity. Next, all mice were transduced with adeno-associated adenovirus serotype 9 (AAV9) expressing a cre-inducible, endoplasmic reticulum-restricted TurbolD (AAV9-FLEx-ER-TurbolD, 3*10e11 GC/mouse, intravenously) (see Methods). AAV9 was chosen since this serotype exhibits broad tissue distribution and transduction (Zincarelli et al., 2008). In the cases where tamoxifen-inducible cre driver lines were used, tamoxifen (2 mg/mouse, intraperitoneally) was administered two weeks after viral transduction. As expected, robust TurbolD mRNA expression was detected in tissues from all transduced mice compared to virally-transduced, wild-type control mice (FIG. 7A and see Methods). Furthermore, we harvested a collection of tissues from a subset of cre driver mice in which TurbolD expression was predicted to occur in an organ-restricted manner (Albumin-cre for liver, Ucp1-cre for brown fat, Adiponectin-cre for all adipose, MCK-cre for muscle, Myh6-cre for heart, Pdx1-cre for pancreas, and Syn1-cre for brain). V5-tagged TurbolD protein, as measured by Western blot using an anti-V5 epitope tag antibody, was robustly detected with the expected organ-restricted pattern (FIG. 7B).

Three weeks after viral transduction, mice were separated into treadmill running or sedentary groups (N=3 per group per genotype, FIG. 1A). Our one-week treadmill running protocol, adapted from (Wu et al., 2011), consisted of running for 60 min/day at a speed of 20 m/min (see Methods). Sedentary mice were kept in their home cages. After the one-week treadmill training protocol, mRNA levels of Pgc1a and Nr4a1 in quadriceps muscle were significantly induced (P<0.001) and (P<0.001), respectively (FIG. 7C-7D) (Finck and Kelly, 2006; Kanzleiter et al., 2009; Kawasaki et al., 2009). Total inguinal white adipose tissue mass was reduced in the exercise group (P<0.001) (FIG. 7E). Histological analysis of the inguinal adipose tissue also showed reduced adipocyte size (FIG. 7F). Additionally, all exercised animals exhibited −0.68±0.02 g (mean±SEM, N=66) weight change during the 1-week treadmill running whereas the sedentary controls (N=66) gained+0.50±0.02 g (mean±SEM, N=66, FIG. 7G). Food intake increased in the exercise group (FIG. 7H).

Taken together, these molecular and physiologic data validate both the secretome labeling mouse lines as well as the one-week exercise training protocol.

To determine the identity of exercise-regulated secreted proteins and their cell types of origin, we supplemented biotin to mice for the final three days of the exercise training protocol to biotinylate in vivo secretomes (FIG. 1A, right, FIG. 71, and see Methods). Two hours after the final bout of running, blood plasma was collected from each mouse and biotinylated secreted proteins were purified using streptavidin beads, digested following an S-trap protocol, and analyzed by LC-MS/MS in data-independent acquisition (DIA) mode (see Methods). We chose to use a previously described spectrum library-free DIA approach that relies on gas-phase fractionation (GPF)-DIA data from a pooled sample to generate DIA-only chromatogram libraries (Pino et al., 2020a; Searle et al., 2018). This allowed us to search all data against experiment-specific chromatogram libraries using the freely available EncyclopeDIA platform (Searle et al., 2018), followed by further processing in the Skyline and Perseus data analysis environments (Pino et al., 2020b; Tyanova et al., 2016). To filter for bona fide cell type-protein pairs enriched by streptavidin, we also applied a 1.5-fold enrichment filter for each cell type-protein pair versus non-transduced, wild-type controls (see Methods). In total across all samples (N=3 mice/condition×2 conditions×21 genotypes), we detected 1,272 unique cell type-protein pairs with ≥2 peptides detected in all 3 replicates of both conditions.

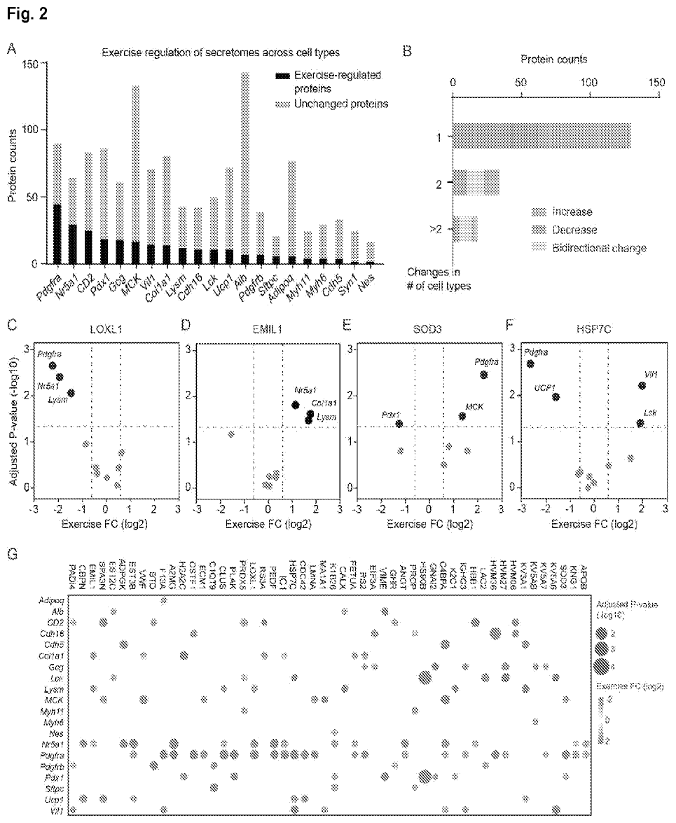

Exercise-regulated cell type-protein pairs were identified by comparison of differential secreted proteins from the same cell type in sedentary versus exercised mice. This comparison also provides a natural control for differing levels of proximity labeling enzyme expression and/or varying levels of secretome biotinylation between distinct cell types. Exercise significantly altered 256 cell type-protein pairs (20.1% of the entire dataset, adjusted β-value <0.05, FIG. 2A). The distribution of these exercise-regulated cell type-protein pairs across the 21 cell types is shown in FIG. 1B. We observed an average and median of 12 and 10, respectively, proteins changed per cell type secretome in response to exercise, with a range across all cell types of 1 to 44. The number of proteins changed in each cell type secretome by exercise was not simply correlated to secretome size (FIG. 2A) since the two cell types with the largest number of unique proteins identified in their secretomes (Albumin-cre, with 142 proteins, and MCK-cre, with 132 proteins) exhibited near the median number of exercise-regulated secreted protein changes (FIG. 2A). All cell types exhibited some secretome changes following exercise, demonstrating that all cell types exhibit exercise responsiveness to some degree as measured by changes to their production of soluble factors. We conclude that one week of treadmill running in mice results in specific modulation of a subset of secreted proteins-cell type pairs across our entire secretome dataset.

### 2. Systematic Analysis of Exercise-Regulated Cell Type-Protein Pairs

A volcano plot showing the 1,272 cell type-protein pairs detected in this experiment is shown in FIG. 1B. Approximately half of the exercise-regulated secretome changes (50%, 129 out of 256) were observed in only one cell type. In addition, the frequency of either exercise up- or down-regulated proteins was equivalent (66 proteins increased and 63 proteins decreased, FIG. 2B), suggesting that exercise regulation of soluble factors does not only involve production of new secreted molecules, but also suppression and other regulation of active protein secretion. The other half of the exercise-regulated secretome changes (50%, 127 out of 256) were proteins expressed in multiple cell types that exhibited cell type-specific regulation following exercise. In this latter group, bidirectional change, defined as up-regulation in one cell type and down-regulation in a different cell type, was commonly observed (40%). These data further underscore the increased resolution afforded by cell type-specific secretome profiling, as well as the need to use global approaches for evaluating exercise-induced changes, which are complex, bidirectional, and cell type-specific.

We identified several example candidates of exercise-regulated secreted proteins selectively altered in one cell type, the majority of which were not previously reported to be regulated by physical activity. For instance, two carboxylesterases, CES2A and CES2C, were increased by 3-fold exclusively in secretomes from Albumin-cre mice following treadmill running (FIGS. 1C and 1D). CES2 enzymes are classically annotated as intracellular, liver-enriched endoplasmic reticulum resident proteins. Nevertheless, our data suggest that CES2 enzymes can also be released from the liver in an exercise-dependent manner. Similarly, the most exercise-inducible protein in secretomes from Pdgfra-cre transduced mice was TIMP3 (tissue inhibitor of metalloproteinases 3) (FIG. 1F), a secreted protein with diverse physiologic roles including in myogenesis (Liu et al., 2010), thermogenesis and metabolism (Hanaoka et al., 2014), vascular remodeling (Basu et al., 2013), and atherogenesis (Stöhr et al., 2014). On the other hand, several other well established secreted protein-cell type pairs, which include the hormones adiponectin (ADIPOQ, from Adipoq-cre secretomes) (Stern et al., 2016) and fetuin B (FETUB, from Albumin-cre secretomes) (Meex et al., 2015), were robustly detected in our dataset but not regulated by physical activity (FIG. 1E, FIG. 13).

Direct examination of the exercise-regulated secreted protein changes of proteins that were expressed across multiple also revealed previously unknown and unusual cell type-specific patterns of exercise regulation. For instance, LOXL1, a secreted enzyme involved in extracellular protein lysine oxidation, was selectively downregulated following exercise in secretomes from Pdgfra-, Nr5a1-, and Lysm-cre transduced mice (FIG. 2C). Conversely, the extracellular matrix protein EMIL1 was upregulated following exercise selectively in Colla1, Lysm and Nr5a1-cre secretomes (FIG. 2D). We also identified several examples of proteins expressed in multiple cell types and bidirectionally regulated by exercise. These include SOD3 (upregulated in MCK-cre and Pdgfra-cre, and downregulated in Pdx1-cre secretomes) (FIG. 2E) and HSP7C (upregulated in Vil1-cre and Lck-cre, and downregulated in Pdgfra-cre and UCP1-cre secretomes) (FIG. 2F). A bubble plot visualizing all such examples of exercise-regulated secreted proteins with regulation in >2 cell type secretomes is shown in FIG. 2G. While several of these proteins have been identified as exercise-regulated proteins in the literature, our dataset further contextualizes and refines their interpretations at a cell type level. For instance, total plasma SOD3 was previously reported to be induced by acute treadmill running in humans as well as voluntary wheel running and treadmill exercise in mice (Abdelsaid et al., 2022; Fukai et al., 2000; Hitomi et al., 2008). However, only one of these previous studies suggested that muscle is a cellular origin for exercise-inducible SOD3 (Hitomi et al., 2008). Our dataset not only confirms upregulation of SOD3 secretion from muscle following exercise, but also identifies Pdgfra and Pdx1 as additional cell types that express SOD3 and contribute to the exercise-inducible regulation of this protein (FIG. 2E). Similarly, PEDF is a neurotrophic growth factor widely expressed across multiple cell types. In humans, circulating PEDF is reduced after a single bout of cycling (Raschke et al., 2013) as well as after 12-month moderate-intensity aerobic exercise (Duggan et al., 2014). PEDF was downregulated in our dataset in both Nr5a1-cre and Pdgfra-cre secretomes (FIG. 2G), suggesting that these two cell types contribute to the downregulation of plasma PEDF after exercise.

3. Secretomes from Pdgfra-Expressing Cells are Highly Responsive to Exercise Training

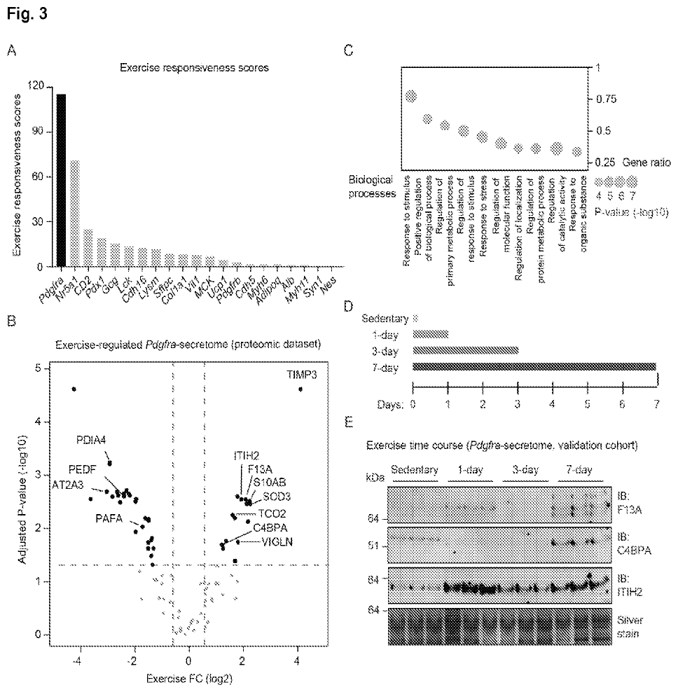

Secretomes from metabolic tissue cell types (myocytes, hepatocytes, and adipocytes) have been most well studied in the literature in the context of exercise regulation. By counting the number of exercise-regulated secreted proteins (adjusted β-value <0.05), we found that myocyte, hepatocyte, and adipocyte secretomes ranked 6th, 13th, and 16th, in terms of exercise responsiveness, respectively, out of the 21 cell types examined (FIG. 2A). In the muscle secretome, specific secreted proteins contributing to the exercise-responsiveness metric included C1QA (3-fold increased), SOD3 (3-fold increased), and VWF (4-fold decreased). Similarly, we identified H6PD (7-fold increased) and AMY1 (3-fold increased) as contributors to the exercise responsiveness metric in the adipose secretome, and CES2A (3-fold increased) and CES2C (3-fold increased) in the liver secretome. As an alternative method for determining magnitude of the exercise response, we also developed a direct “exercise-responsiveness” metric for each cell type secretome in our dataset (see Methods and FIG. 3A). This metric takes into account magnitude and statistical significance of the exercise-regulated changes, as well as the secretome size of that cell type. Myocyte, adipocyte, and hepatocyte, secretomes scored 6.9, 1.1, and 0.9, respectively, using this exercise-responsive metric, again ranking 13th, 17th, and 18th, respectively, out of the 21 cell types examined. Therefore these three metabolic tissue secretomes are regulated by physical activity, but are neither highly responsive nor highly unresponsive to exercise relative to the other cell types secretomes examined here.

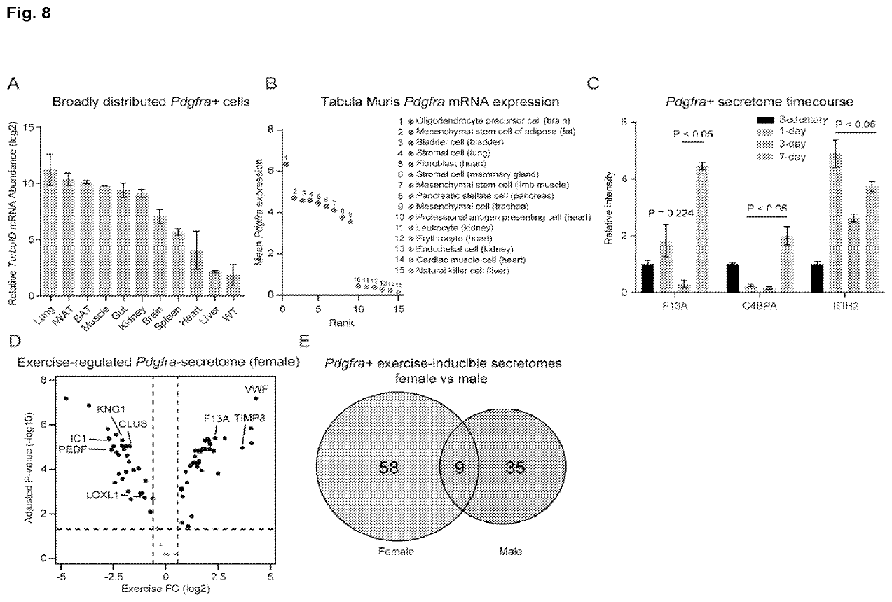

Surprisingly, Pdgfra-cre secretome exhibited the highest exercise responsiveness across all the cell type secretomes, regardless of the metric used (rank 1/21 with 44 exercise-regulated secreted proteins, and also rank 1/21 with exercise-responsiveness score=115.4). Pdgfra-cre labeled cells are a population of anatomically-distributed cells that have been described in the literature as fibroblasts, mesenchymal stem cells, or progenitor/precursor cells (Endale et al., 2017; Green et al., 2016; Li et al., 2018; Merrick et al., 2019; Ntokou et al., 2015; Zepp et al., 2017). They have diverse roles in tissue remodeling, fibrosis and cell proliferation depending on the resident tissue and physiological context (Li et al., 2018; Zepp et al., 2017). To understand the organ localization of the Pdgfra-cre labeled cells labeled in our secretome labeling experiments, we measured TurbolD mRNA across multiple tissues from Pdgfra-cre transduced mice (FIG. 8A). Robust TurbolD mRNA enrichment was detected across many tissues examined, including lung, adipose tissues (inguinal and brown), muscle, gut, kidney, and brain (FIG. 8B). This distribution is similar to the reported expression of Pdgfra mRNA across tissues and suggest that Pdgfra localized to multiple organs are participating in the exercise-regulated secretome response detected in our dataset.

The entire secretome of Pdgfra-cre labeled cells is shown in FIG. 3B. A diverse array of secreted proteins with pleiotropic physiologic functions were found to be regulated by exercise. Some of the most upregulated molecules included the previously mentioned TIMP3 (17-fold) and SOD3 (5-fold), as well as the S100 family member S10AB (5-fold), the HDL-binding protein VIGLN (4-fold), and the vitamin B12 transport protein TCO2 (3-fold). Conversely, downregulated secreted proteins in the Pdgfra-cre secretome included ER-resident proteins such as the protein disulfide isomerase PDIA4 (87% suppression) and ER calcium ATPase AT2A3 (88% suppression), the growth factor PEDF (81% suppression), and the lipid metabolizing enzyme PAFA (69% down). Gene ontology analysis revealed enrichment of several biological pathways in the exercise-regulated Pdgfra-cre secretome, of which the highest scoring by gene ratio was “response to stimulus” (P=4.96E-07, gene ratio=0.77). Additional biological processes identified from the Pdgfra-cre secretome included “response to stress” (P=2.00E-06, gene ratio=0.45), and “response to organic substance” (P=1.03E-04, gene ratio=0.34) (FIG. 3C). These observations suggest that Pdgfra-cre labeled cells respond to exercise by sensing exercise-regulated environmental cues, such as metabolites, cytokines, or other signaling molecules, that in turn drive bidirectional changes in the Pdgfra-cre labeled secretome. The exercise-regulated Pdgfra-cre labeled secretome of female mice is depicted in FIG. 8D. Overlap between exercise-regulated cell type-protein pairs of female and male Pdgfra secretomes is depicted in FIG. 8E.

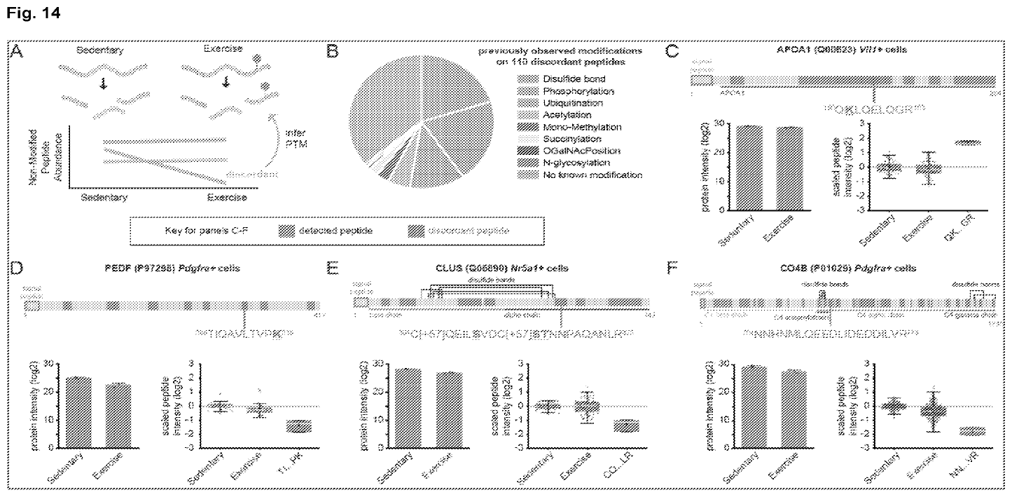

We next sought to validate some of the Pdgfra-cre secretome responses using an orthogonal method, as well as to determine whether the secretome changes of Pdgfra-labeled cells represented an acute or chronic response to physical activity. Towards this end, we used Western blotting with commercially available antibodies to determine the levels of three exercise-regulated secreted proteins (F13A, C4BPA and ITIH2) from the Pdgfra-cre secretome in a new cohort of virus-transduced, Pdgfra-cre mice. In our original proteomic dataset, F13A, C4BPA, and ITIH2 were found to be 4-, 2-, and 4-fold upregulated in Pdgfra-cre secretomes after 1 week treadmill running. In this experiment, transduced animals (N=5/group) were separated into three groups: sedentary, acute exercise (single treadmill bout, 20 m/min for 60 min), 3 day or 1-week chronic exercise (daily treadmill running, 20 m/min for 60 min) (FIG. 3D). As shown in FIG. 3E and FIG. 8C, Western blotting revealed that two of the three proteins, F13A and ITIH2, were elevated from Pdgfra-cre secretomes in both the acute as well as chronic treadmill running cohorts. Interestingly, while ITIH2 exhibited a similar magnitude upregulation after 1- or 7-days running, the upregulation of F13A was higher as the exercise duration increased (e.g., 7-day>1 day>sedentary). On the other hand, C4BPA was down-regulated at 1 day, and upregulated by 7-days, indicating this protein is suppressed after acute exercise but increased after chronic training. Importantly, the total eluted proteins from streptavidin purification remained constant. These data demonstrate that the exercise-regulated proteins in the Pdgfra-cre secretome are robust across multiple cohorts and reflect both acute as well as chronic aspects of exercise training. 4. Exercise induces cell type-specific regulation of secreted proteoforms Proteoforms (Aebersold et al., 2018; Smith et al., 2013), or different molecular forms of a protein product derived from a single gene, are the effector molecules of biological function. While scattered examples of exercise-regulated proteoforms have been reported in the literature (Boström et al., 2012; Hyatt and Powers, 2020; Kurgan et al., 2019; Parker et al., 2017), the extent to which this occurs has yet to be systematically investigated. We reasoned that all peptides for a given protein would exhibit similar exercise regulation if only one proteoform existed; however, a discordantly regulated peptide that differs in its abundance from other peptides derived from the same protein may indicate proteoform regulation by physical activity. We therefore used Peptide Correlation Analysis (PeCorA) (Dermit et al., 2021) to fit linear models that assess quantitative disagreements between peptides mapped to the same protein for a given cell type (FIG. 14A). For peptides identified as discordant, we manually inspected PhosphoSitePlus (Hornbeck et al., 2015) and Uniprot (Consortium, 2021) to understand which amino acid residues had been previously observed as modified. Importantly, this discordant peptide methodology enables a wider inference of exercise-regulated proteoforms without the need for apriorienrichment of any specific type of peptide modification.

We first filtered within PeCorA to removed peptides with incomplete data (i.e., any missing values across all six samples of a given cell type, see Methods). In total, 15,712 cell type-peptide pairs were used for PeCorA analysis. Of these, a total of 110 cell-type peptide pairs (representing 0.7% of the dataset) were identified as discordant relative to other peptides from that same protein in that cell type (adjusted β-value <0.05, FIG. 14B). Slightly over half of these peptides carried previously observed site-specific PTMs (60/110, ˜55%), with the top three most prevalent inferred modifications being redox cysteine (25%), phosphorylation (25%) and ubiquitination (15%). The other 50 discordant peptides did not carry a modification that could be mapped to PhosphoSitePlus or Uniprot. Nevertheless, our detection of their discordant abundance in our dataset suggests that they may harbor modifications that are not currently annotated in any public database.

FIG. 14C-F highlight several representative examples of cell type-protein pairs with discordant peptides from which an exercise-regulated proteoform could be inferred. FIG. 14C shows a discordant peptide, QKLQELQGR (SEQ ID NO:29), identified from the Vil1-cre/APOA1 cell type-protein pair. This discordant peptide contains a well-documented acetyl-lysine site K156 whose modification status has previously been reported to be regulated by fasting and feeding (Yang et al., 2011). That this discordant peptide is increased suggests that exercise induces a potential lysine deacetylation modification of gut-derived APOA1. FIG. 14D shows a discordant peptide TIQAVLTVPK (SEQ ID NO:30) that was identified in the Pdgfra-cre/PEDF cell type-protein pair. In this case, the K315 residue is annotated as a site for mono-methylation. Here, however, the decrease in abundance of the unmodified discordant peptide suggests that exercise regulation of PEDF in Pdgfra-cre secretomes involves potential methylation of this residue. A third discordant peptide in Nr5a1-cre/CLU (CQEILSVDCSTNNPAQANLR; SEQ ID NO:31) spans multiple known phosphosites (FIG. 14E). The down regulation of the discordant peptides indicates that one or more of these potential sites may become phosphorylated upon exercise. Lastly, not all discordant peptides identified via PeCorA correspond to reversible, site-specific modifications. For instance, the Pdgfra-cre/C04b cell type-protein pair was observed to have a discordant peptide that maps to the cleavage site between the bioactive C4a anaphylatoxin and the remainder of the complement C4 alpha chain (FIG. 14F). These data are suggestive of potential exercise-regulated proteolytic processing of complement C4 in Pdgfra+ cells. Overall, our ability to infer several examples of exercise-regulated proteoforms using discordant peptide analysis suggests that non-canonical proteoforms may be more widespread mediators of tissue crosstalk in exercise than previously appreciated.

### 5. In Vitro Studies of Exercise-Inducible CES2 Secretion

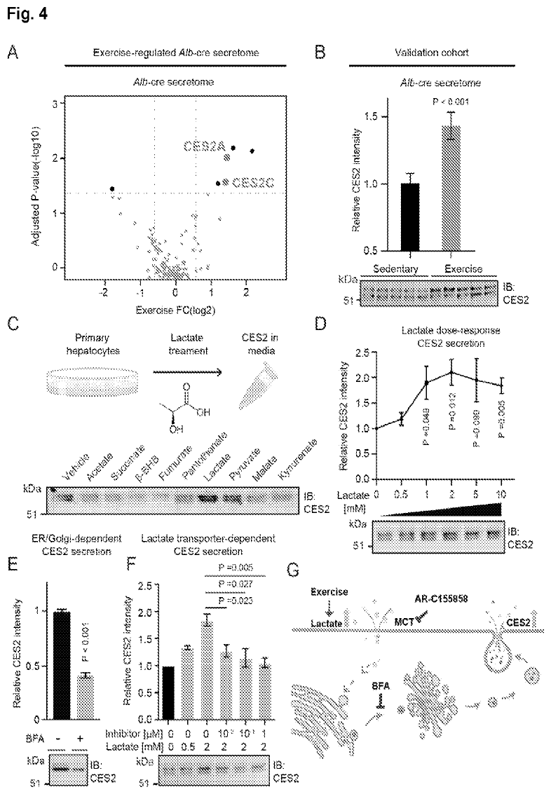

Two of the most robust exercise-inducible molecules from Albumin-cre secretomes belonged to the same family of carboxylesterase enzymes (CES2A and CES2C, FIG. 4A). Because hepatocytes can be easily cultured and manipulated in vitro, we used the Albumin-cre/CES2 cell type-protein pair as a representative molecular handle to investigate the molecular drivers of this process in vitro.

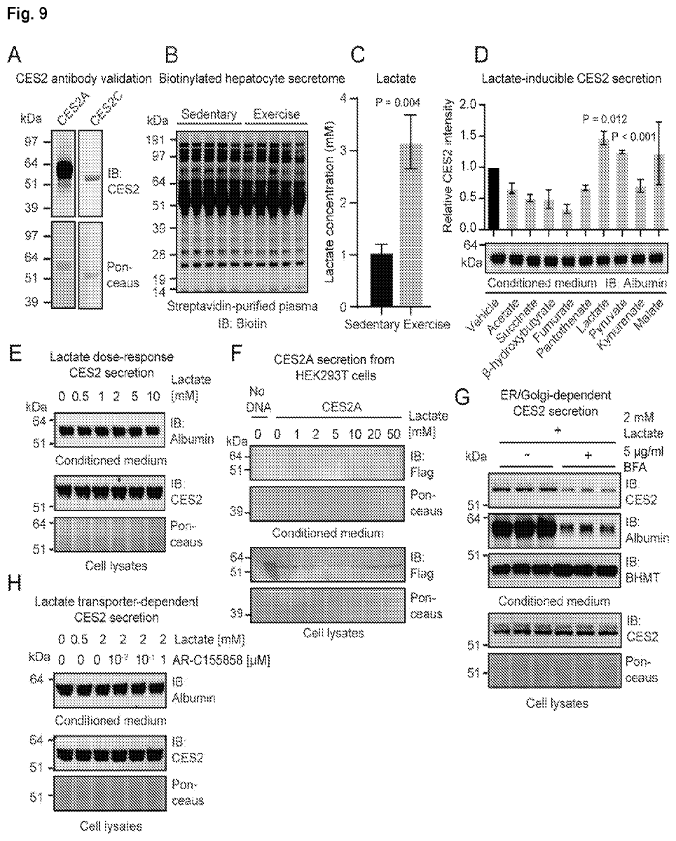

To first validate the exercise-inducible secretion of CES2 proteins from the liver, we used a commercially available pan anti-CES2 antibody to probe streptavidin-purified blood plasma from a separate cohort of TurbolD-transduced and exercised Albumin-cre mice. This commercially available anti-CES2 antibody exhibited the expected immunoreactivity to purified, recombinantly produced CES2A and CES2C proteins (FIG. 9A). As expected, extracellular CES2 levels from streptavidin-purified Albumin-cre secretomes were increased after exercise. The total hepatocyte secretome biotinylation signal remained unchanged (FIG. 4B), establishing equivalent secretome protein loading. We therefore conclude that CES2 secretion from the liver is a robust molecular event in response to one week of treadmill running. A western blot of the biotinylated hepatocyte secretomes of exercised and sedentary mice is depicted in FIG. 9B.

Circulating lactate increased in exercised mice compared to sedentary mice (P=0.004) (FIG. 9C). We sought to test the hypothesis that lactate might serve as an exercise-inducible extracellular signal that stimulates CES2 secretion from hepatocytes. This hypothesis was based on the well-established increase in lactate flux through the liver via the Cori cycle, as well as our previous experiments showing that other metabolic fuels (e.g., fatty acids) can stimulate protein secretion from the liver (Wei et al., 2020).Primary hepatocytes were treated with lactate (2 mM, 4 h) and extracellular CES2 proteins were measured by Western blotting in both cell lysates and conditioned medium. As additional controls, we tested a variety of other exercise-regulated organic acids, including pyruvate, acetate, malate, fumarate, beta-hydroxybutyrate, kynurenate, and pantothenate (Agudelo et al., 2014; Contrepois et al., 2020; Reddy et al., 2020; Sato et al., 2022; Schranner et al., 2020). As shown in FIG. 4C and FIG. 9D, lactate treatment robustly increased the levels of extracellular CES2. Pyruvate, a structurally similar metabolite, also increased CES2 secretion, though with a slightly lower magnitude than that of lactate. By contrast, none of the other metabolites tested increased extracellular CES2 levels (FIG. 4C, FIG. 9D), establishing that only extracellular lactate, and to a lesser extent pyruvate, exhibit CES2 secretion stimulatory activity.

A dose response of lactate revealed increased CES2 secretion with exogenous lactate treatment even as low as 0.5 mM (FIG. 4D), whereas intracellular CES2 levels were unchanged at all concentrations of lactate tested. In addition, the effect of lactate to induce secretion of CES2 was specific since extracellular albumin levels were unchanged with lactate treatment (FIG. 4D, FIG. 9E). This lactate-induced CES2 secretion is cell type-specific since exogenous expression of CES2A in HEK293T cells resulted in complete retention of this protein intracellularly and treatment of lactate (1-50 mM, 4 h) concentration did not induce the release of CES2A into conditioned medium (FIG. 9F). To understand if CES2 secretion is via ER-Golgi secretory pathway, we treated primary hepatocytes with the vesicle transport inhibitor Brefeldin A (BFA). This results in intracellular retention of secreted proteins with N-terminal signal peptide. As expected, BFA treatment (5 μg/ml, 4 h) dramatically decreased classically secreted albumin while not interfering with the unconventional export of a cytosolic protein BHMT (Wei et al., 2020) from hepatocytes (FIG. 4E, FIG. 9G). As shown in FIG. 4E, BFA treatment blocked lactate-induced increase in extracellular CES2 fraction. Because import into hepatocytes is critical for lactate to function as a substrate in the Cori cycle, we next tested whether lactate import via the monocarboxylate transporters (MCTs) was required for induction of CES2 protein secretion. Treatment of primary hepatocytes with AR-C155858, a nanomolar dual MCT1/2 inhibitor (Ovens et al., 2010a; Ovens et al., 2010b), dose-dependently inhibited the lactate-induced secretion of CES2 (FIG. 4F). Once again, the inhibitory effect of AR-C155858 was selective for CES2, since no changes were observed in extracellular albumin under these conditions (FIG. 4F, FIG. 9H). A model of CES2 secretion from cells where exercise training inducible rise of extracellular lactate induces release of ER-lumen-resident CES2 from hepatocytes and functional lactate transporters and ER-Golgi vesicle transport are required for CES2 secretion is depicted in FIG. 4G. These data demonstrate that extracellular lactate is sufficient to drive secretion of CES2 proteins via classical pathway from hepatocytes in a manner that requires import of lactate into hepatocytes.

### 6. Soluble CES2 Proteins Exhibit Anti-Obesity and Anti-Diabetic Effects in Mice

Finally, we sought to determine whether release of extracellular CES2 from the liver following exercise was simply a response to exercise training, or whether soluble CES2 proteins might function as circulating molecular effectors of physical activity. Supporting a potential functional role for extracellular CES2, three prior studies showed that liver-specific overexpression of either human or mouse CES2 lowered body weight, reduced hepatic steatosis, and improved glucose homeostasis (Li et al., 2016; Ruby et al., 2017; Xu et al., 2021). An intestine-specific transgenic CES2C mouse model also exhibited a similarly improved metabolic phenotype (Maresch et al., 2019). However, these prior studies did not consider the possibility that extracellular CES2, which is likely also increased in addition to elevation of intracellular CES2 in these transgenic models, might in part mediate the anti-obesity, anti-steatosis, and anti-diabetic phenotypes observed.

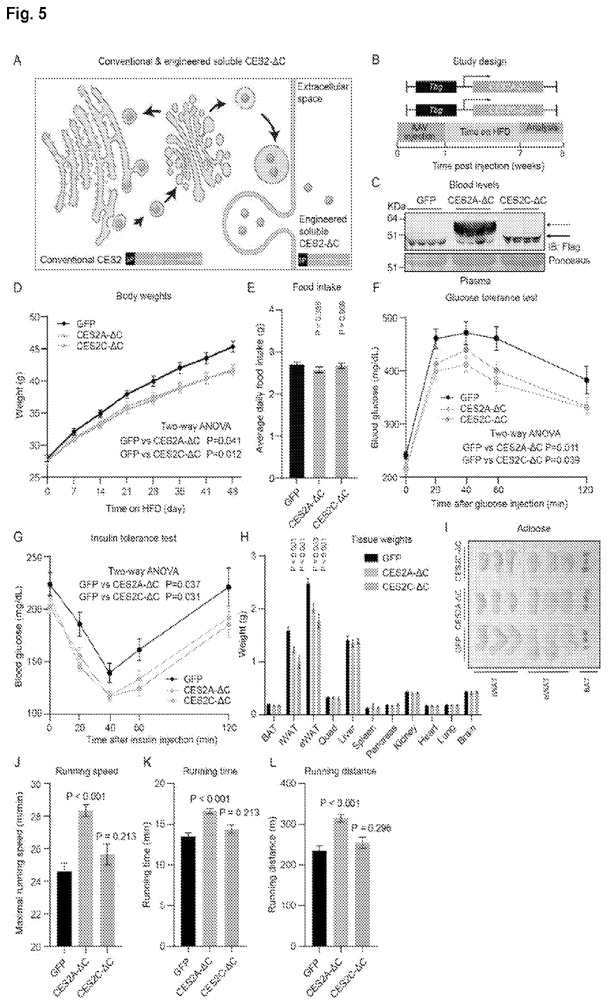

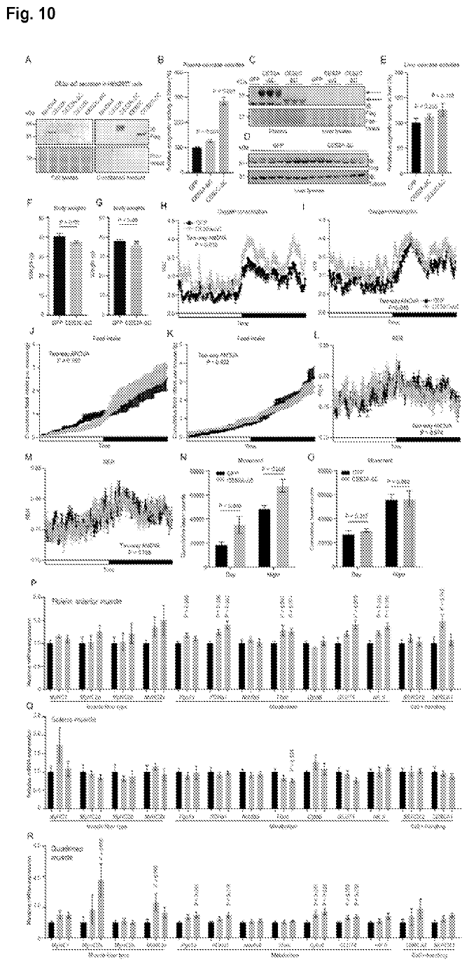

To directly test the functional role extracellular CES2 in energy metabolism and glucose homeostasis without a confounding contribution from intracellular CES2, we set out to generate an engineered version of CES2 that would be exclusively localized extracellularly. Analysis of the primary amino acid sequences for both murine CES2A and CES2C proteins revealed an N-terminal signal peptide, a central alpha/beta hydrolase superfamily domain with the catalytic active site GXSXG motif, and a C-terminal HXEL motif (X=A for CES2A, and R for CES2C). Previous studies showed that the C-terminal HXEL motif is indispensable for the ER lumen localization and C-terminal deleted version of CES2 can be readily detectable in the conditioned medium of cancer cells (Hsieh et al., 2015; Oosterhoff et al., 2005; Potter et al., 1998). We therefore generated CES2A/C constructs in which the HXEL amino acids were removed from the C-terminus (CES2-ΔC) (FIG. 5A). An additional N-terminal Flag epitope tag was included after the signal peptide to aid downstream detection. Both wild-type CES2 and CES2-ΔC constructs were transfected into HEK293T cells and the CES2 protein localization was determined by Western blotting of cell lysates and conditioned media. As expected, full-length CES2A/C were enriched intracellularly, whereas both CES2A-ΔC and CES2C-ΔC proteins were exclusively found extracellularly (FIG. 10A).

To deliver the engineered soluble CES2 proteins to mice, we generated adeno-associated virus (serotype 8) expressing each of our two engineered CES2A-ΔC and CES2C-ΔC constructs under the control of the hepatocyte-specific thyroxine binding protein (Tbg) promoter (FIG. 5B). Next, mice were transduced with AAV-Tbg-CES2A-ΔC or AAV-Tbg-CES2C-ΔC (N=10/group, 10e11 GC/mouse, intravenously). Control mice were transduced with an equal titer of AAV-Tbg-GFP. As expected, Western blotting of blood plasma using an anti-Flag antibody revealed elevation of circulating CES2A-ΔC and CES2C-ΔC (FIG. 5C, FIG. 10C), which was further validated by measuring plasma ester hydrolysis activity using a previously reported synthetic substrate of carboxylesterases (FIG. 10B). In contrast to blood plasma, we did not observe significant changes in total liver CES2 protein level as shown by the anti-CES2 antibody staining (FIGS. 10C and 10D) nor an increase in liver ester hydrolysis activity (FIG. 10E). These data confirm that our viral constructs increase only extracellular CES2A and CES2C levels without affecting the intracellular levels of CES2.

One week after viral transduction, mice were placed on high-fat diet (HFD, 60% kcal from fat). Over the subsequent 7 weeks, both CES2A-ΔC and CES2C-ΔC groups of mice exhibited reduced body weight compared to mice transduced with AAV-Tbg-GFP (GFP: 45.4±0.8 g versus CES2A-ΔC 41.5±0.9 g and CES2C-ΔC 41.8±0.8 g, mean±SEM) (FIG. 50). Body weights of CES2A-ΔC and CES2C-ΔC groups of mice compared to GFP group of mice at 5 or 3 weeks are shown in FIGS. 10F and 10G, respectively. Food intake over this time period was unaltered, suggesting that the lower body weights are not simply due to reduced caloric intake (FIG. 5E, FIG. 10J, and FIG. 10K). In the 7th week, glucose and insulin tolerance tests revealed improved glucose clearance and insulin sensitivity in both CES2A-ΔC and CES2C-ΔC groups (FIG. 5F and G). Dissection of tissues at the end of the experiment revealed significant reductions of inguinal white adipose tissue (iWAT) (23% and 38% reduction for CES2A-ΔC and CES2C-ΔC, respectively, vs GFP) and epididymal adipose tissue (eWAT) mass (18% and 29% reduction for CES2A-ΔC and CES2C-ΔC, respectively, vs) (FIG. 5H and I). The lean mass of all three groups remained unchanged (FIG. 5H), establishing the effects on body weight are due to reduced adiposity and not any changes in lean mass. Maximal running speed (FIG. 5J), total running time (FIG. 5K) and total running distance (FIG. 5L) of 16 to 18-week-old male C57BL/6 mice 8 weeks after being injected with AAV-Tbg-CES2A-ΔC, AAV-Tbg-CES2C-ΔC or AAV-Tbg-GFP (N=8-10/group, 10e11 GC/mouse, intravenously) were tested. The CES2A-ΔC mice group exhibited improved maximal running speed (P<0.001, FIG. 5J), total improved running time (P<0.001 FIG. 5K) and improved total running distance (P<0.001, FIG. 5L). Oxygen consumption of CES2A-ΔC and CES2C-ΔC mice groups increased compared to the GFP mice group (P=0.010 and P=0.045, respectively) (FIG. 10H and 10I, respectively). Respiratory exchange ratio (RER) did not differ in CES2A-ΔC and CES2C-ΔC mice groups compared to the GFP mice group (FIGS. 10L and 10M, respectively). Both day and night movement increased in the CES2A-ΔC mice group compared to the GFP mice group (P=0.043 and P=0.006, respectively) (FIG. 10N), but not in the CES2C-ΔC mice group compared to the GFP mice group (FIG. 10O). CES2A-ΔC and CES2C-ΔC mice groups revealed changes in gene expression of muscle fiber type, metabolism, and Ca²⁺ handling genes across Tibialis anterior, Soleus, and Quadricep muscles compared to the GFP mice group (FIG. 10P, 10Q, and 10R, respectively).

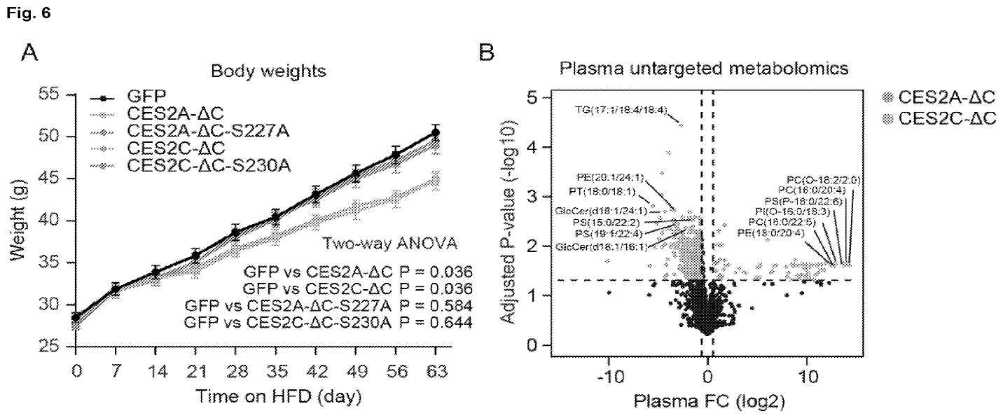

Mice groups transduced with mutant CES2A-ΔC (S227A) or CES2C-ΔC (S230A) lacking carboxylesterase enzymatic activity do not exhibit reduced body weights compared to a mice group transduced with GFP (P=0.584 and P=0.644, respectively) (FIG. 6A). Untargeted metabolomic measurements in blood plasma of CES2A-ΔC and CES2C-ΔC mice reveal significantly increased and decreased blood plasma features compared to GFP mice (FIG. 6B).

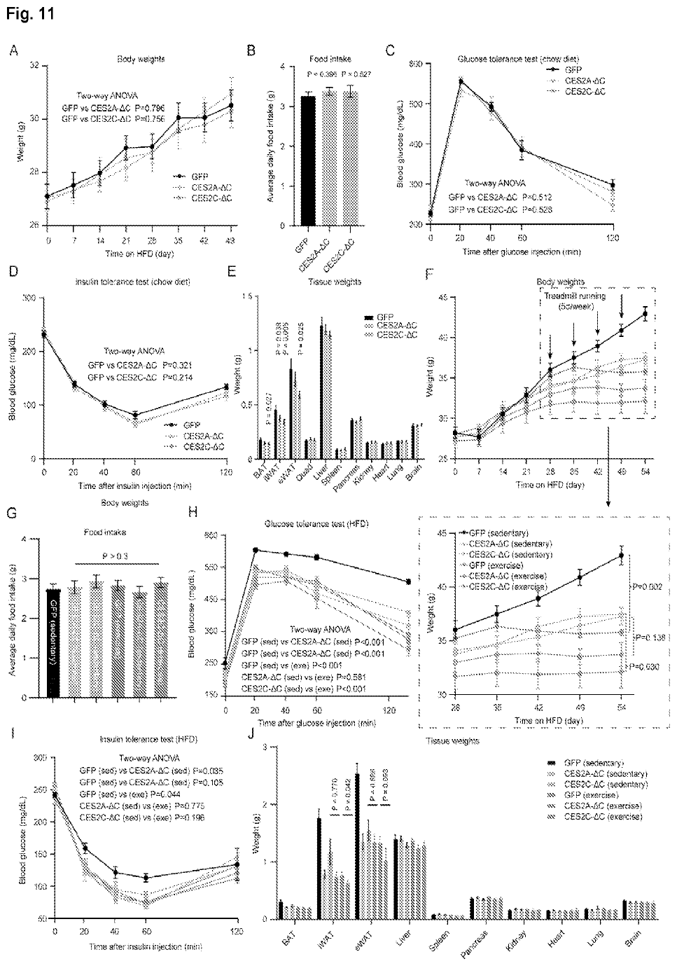

For mice fed a chow diet, the body weights of CES2A-ΔC and CES2C-ΔC mice groups compared to a GFP mice group did not differ over the first 7 weeks of chow diet feeding (FIG. 11A). Food intake over this time period was unaltered (FIG. 11B). Glucose and insulin tolerance tests revealed similar glucose clearance and insulin sensitivity in both CES2A-ΔC and CES2C-ΔC mice groups vs the GFP mice group under a chow diet (FIG. 11C and D). Dissection of tissues at the end of the experiment revealed reductions of inguinal white adipose tissue (iWAT) (P=0.038 and P=0.005 for CES2A-ΔC and CES2C-ΔC, respectively, vs GFP), brown adipose tissue (BAT) (P=0.027 for CES2C-ΔC, vs GFP), and epididymal adipose tissue (eWAT) mass (P=0.025 for CES2C-ΔC, vs GFP) under a chow diet (FIG. 11E). The lean mass of all three groups remained unchanged (FIG. 11E), establishing the effects on body weight are due to reduced adiposity and not any changes in lean mass.

For mice fed a high fat diet with or without exercise (sedentary), the GFP mice group with exercise had significantly reduced body weights compared to the sedentary GFP mice group over the first 7 weeks of high fat diet feeding (P=0.002) (FIG. 11F). Sedentary CES2A-ΔC and CES2C-ΔC mice groups had comparable body weights compared to the GFP mice group with exercise. The CES2C-ΔC mice group with exercise had reduced body weights compared to the sedentary CES2C-ΔC mice group (P=0.030) (FIG. 11F). Food intake was unchanged across experimental groups (FIG. 11G). Glucose tolerance tests revealed improved glucose tolerance in sedentary CES2A-ΔC and CES2C-ΔC mice groups compared to the sedentary GFP mice group (P<0.001 and P<0.001, respectively) (FIG. 11H). Similarly, the GFP mice group with exercise had improved glucose tolerance compared to the sedentary GFP mice group (P<0.001) (FIG. 11H). The CES2C-ΔC mice group with exercise showed improved glucose tolerance compared to the sedentary CES2C-ΔC mice group (P<0.001) (FIG. 11H). Insulin tolerance tests revealed improved insulin tolerance in the GFP mice group with exercise compared to the sedentary GFP mice group (P=0.044) (FIG. 11I). Similarly, the sedentary CES2C-ΔC mice group had improved insulin tolerance compared to the sedentary GFP mice group (P=0.035) (FIG. 11I). Dissection of tissues at the end of the experiment revealed reductions of inguinal white adipose tissue (iWAT) (P=0.042 CES2C-ΔC with exercise vs CES2A-ΔC with exercise) and epididymal adipose tissue (eWAT) mass (P=0.093 CES2C-ΔC with exercise vs CES2A-ΔC with exercise) (FIG. 11J).

Taken together, we conclude that extracellular CES2 proteins have functions in energy balance that are independent of their intracellular roles in triglyceride hydrolysis.

### E. Discussion

Here we have generated an organism-wide proteomic dataset of the cell type-specific secretome responses to one week of treadmill running. This dataset provides several insights of potential importance to our understanding of cell and tissue crosstalk during physical activity, including 1) the demonstration that ˜20% of cell type-protein pairs exhibit complex, bidirectional, and cell type-specific regulation following exercise; 2) the identification of Pdgfra cells as a highly responsive cell type to exercise; 3) evidence that the production of non-canonical secreted proteoforms contributes to exercise-regulated tissue crosstalk; and 4) discovery of secreted CES2 as extracellular enzymes with anti-obesity and anti-diabetic functions.

Classically, muscle has been studied as a principal source of activity-inducible “myokines” that mediate tissue crosstalk in exercise. More recent evidence has expanded this model to include exercise-inducible hepatokines from the liver (De Nardo et al., 2022) and adipokines from the fat (Takahashi et al., 2019). Our studies suggest that many more cell types respond to exercise than previously recognized, including Pdgfra+ cells distributed to multiple organ systems. Recent reports using single cell RNA-sequencing showed that exercise regulation in adipose was most strongly pronounced in adipose stem cells (Yang et al., 2021), which are defined by the expression of Pdgfra+(Shin et al., 2020; Wang et al., 2020b). These adipose-resident fibroblasts may indeed correspond to a subset of the Pdgfra+ cells identified in our secretome profiling dataset. In the future, it will be important to specifically determine which exercise-regulated cell types and in which organs beyond adipose are defined by Pdgfra+ expression.

A particular advantage of our proteomics approach is the ability to define exercise-regulated changes at a peptide level. By conducting a discordant peptide analysis, we identify exercise regulation of secreted proteoforms across multiple cell types. While our current dataset does not enable definitive assignment of the specific protein modification, we are able to leverage public proteomics datasets to infer potential post-translational modifications (Consortium, 2021; Hornbeck et al., 2015). These include single amino acid modifications (e.g., phosphorylation, methylation, ubiquitination) as well as larger scale changes (e.g., proteolytic cleavages or other isoforms) of secreted proteins. Importantly, the proteoforms inferred by our discordant peptide analysis appear to exhibit cell type specificity. For instance, while APOA1 was found to be secreted in three secretomes, the exercise-regulated, acetyl-lysine-containing discordant peptide QKLQELQGR was only observed in the Vil1-cre secretome. In the future, it will be important to use targeted methods to interrogate specific classes of protein modifications. More generally, we suggest that future proteome-wide studies would benefit from including efforts to focus on proteoform-level (rather than peptide-level) measurements to capture holistic information about the secreted molecules that might mediate tissue crosstalk in exercise.

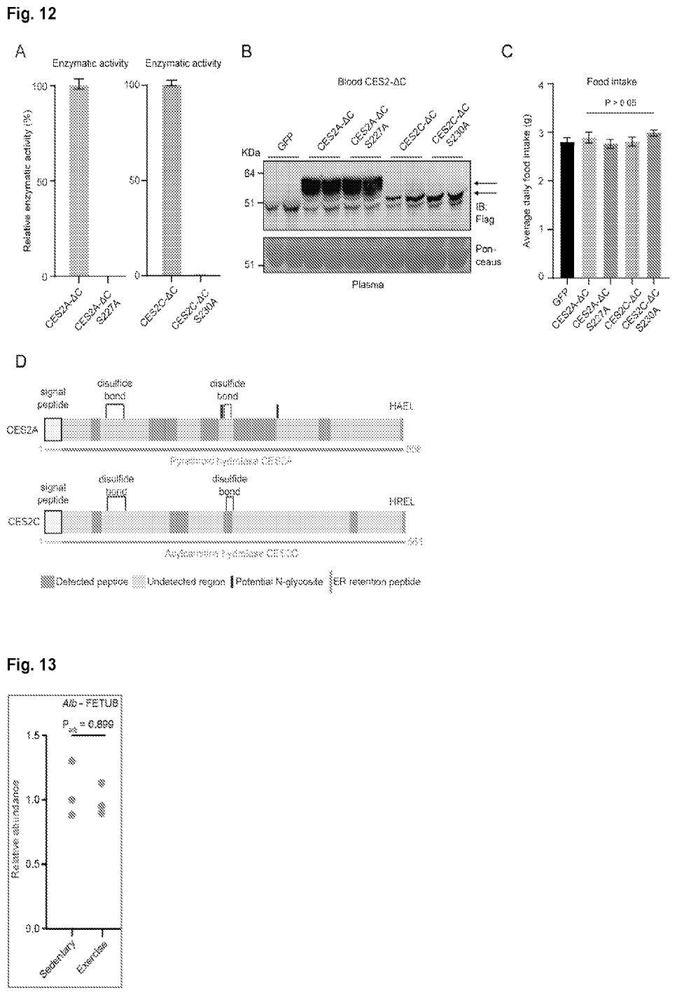

Using cell culture systems, we also provide evidence that the exercise-inducible secretion of CES2 from the liver can be recapitulated by addition of extracellular lactate to primary hepatocytes in vitro. While the precise downstream mechanism linking lactate import to protein secretion from hepatocytes remains unknown, we suspect one likely possibility includes lactate-inducible proteolytic cleavage of the CES2 C-terminus. This C-terminus contains the ER retention signal required for intracellular localization of CES2. In addition, RBBP9 has been proposed as a hydrolase that liberates an array of ER-resident proteins into the extracellular space (Tang et al., 2022). Consistent with this idea, we were unable to detect peptides corresponding to C-terminus of either CES2A or CES2C (FIG. 12D). Such an ER-retention signal cleavage mechanism may also explain the secretion of several other ER-resident proteins in our dataset, such as H6PD (in Adiponectin-cre secretomes), CALX (in Albumin-cre and Lysm-cre secretomes) and AT2A1 (in MCK-cre secretomes). The possibility that lactate itself constitutes a more general mechanism linking physical activity to secreted protein-mediated tissue crosstalk remains an open question for future work.

Lastly, we provide evidence that exercise-inducible secretion of CES2 proteins is not simply a molecular response to exercise. Instead, using engineered versions of CES2 that are localized exclusively extracellularly, we show that soluble CES2 proteins exhibit anti-obesity and anti-diabetic actions in obese mice. Interestingly, the metabolic effects of soluble CES2 proteins are not simply due to reduced caloric intake. We hypothesize that CES2 proteins might regulate some other aspect of energy balance, such as energy expenditure or nutrient absorption. Lastly, the CES2 locus in humans has been linked to multiple cardiometabolic parameters in the UK Biobank, including HDL cholesterol (P=5.14e-311, beta=+0.0625), blood pressure (P=1.64e-17, beta=+0.0317), and BMI-adjusted waist-hip ratio (P=4.61 e-15, beta=−0.0297), suggesting that exercise-regulated soluble CES2 proteins might also impact cardiometabolic health in humans.

### F. REFERENCES

Abdelsaid, K., Sudhahar, V., Harris, R. A., Das, A., Youn, S. W., Liu, Y., McMenamin, M., Hou, Y., Fulton, D., Hamrick, M. W., et al. (2022). Exercise improves angiogenic function of circulating exosomes in type 2 diabetes: Role of exosomal SOD3. FASEB J 36, e22177. 10.1096/fj.202101323R.
Adusumilli, R., and Mallick, P. (2017). Data Conversion with ProteoWizard msConvert. Methods Mol Biol 1550, 339-368. 10.1007/978-1-4939-6747-6_23.
Aebersold, R., Agar, J. N., Amster, I. J., Baker, M. S., Bertozzi, C. R., Boja, E. S., Costello, C. E., Cravatt, B. F., Fenselau, C., Garcia, B. A., et al. (2018). How many human proteoforms are there?Nat Chem Biol 14, 206-214. 10.1038/nchembio.2576.
Agudelo, L. Z., Femenfa, T., Orhan, F., Porsmyr-Palmertz, M., Goiny, M., Martinez-Redondo, V., Correia, J. C., Izadi, M., Bhat, M., Schuppe-Koistinen, I., et al. (2014). Skeletal muscle PGC-1αl modulates kynurenine metabolism and mediates resilience to stress-induced depression. Cell 159, 33-45. 10.1016/j.cell.2014.07.051.
Alvarez-Castelao, B., Schanzenbächer, C. T., Hanus, C., Glock, C., Tom Dieck, S., Dörrbaum, A. R., Bartnik, I., Nassim-Assir, B., Ciirdaeva, E., Mueller, A., et al. (2017). Cell-type-specific metabolic labeling of nascent proteomes in vivo. Nat Biotechnol 35, 1196-1201. 10.1038/nbt.4016.
Amodei, D., Egertson, J., MacLean, B. X., Johnson, R., Merrihew, G. E., Keller, A., Marsh, D., Vitek, O., Mallick, P., and MacCoss, M. J. (2019). Improving Precursor Selectivity in Data-Independent Acquisition Using Overlapping Windows. J Am Soc Mass Spectrom 30, 669-684. 10.1007/s13361-018-2122-8.
Anderson, N. L., and Anderson, N. G. (2002). The human plasma proteome: history, character, and diagnostic prospects. Mol Cell Proteomics 1, 845-867. 10.1074/mcp.r200007-mcp200.
Ashburner, M., Ball, C. A., Blake, J. A., Botstein, D., Butler, H., Cherry, J. M., Davis, A. P., Dolinski, K., Dwight, S. S., Eppig, J. T., et al. (2000). Gene ontology: tool for the unification of biology. The Gene Ontology Consortium. Nat Genet 25, 25-29. 10.1038/75556.
Basu, R., Lee, J., Morton, J. S., Takawale, A., Fan, D., Kandalam, V., Wang, X., Davidge, S. T., and Kassiri, Z. (2013). TIMP3 is the primary TIMP to regulate agonist-induced vascular remodelling and hypertension. Cardiovasc Res 98, 360-371. 10.1093/cvr/cvt067.
Blair, S. N., Kohl, H. W., Paffenbarger, R. S., Clark, D. G., Cooper, K. H., and Gibbons, L. W. (1989). Physical fitness and all-cause mortality. A prospective study of healthy men and women. JAMA 262, 2395-2401. 10.1001/jama.262.17.2395.
Booth, F. W., Roberts, C. K., Thyfault, J. P., Ruegsegger, G. N., and Toedebusch, R. G. (2017). Role of Inactivity in Chronic Diseases: Evolutionary Insight and Pathophysiological Mechanisms. Physiol Rev 97, 1351-1402. 10.1152/physrev.00019.2016.
Boström, P., Wu, J., Jedrychowski, M. P., Korde, A., Ye, L., Lo, J. C., Rasbach, K. A., Boström, E. A., Choi, J. H., Long, J. Z., et al. (2012). A PGC1-α-dependent myokine that drives brown-fat-like development of white fat and thermogenesis. Nature 481, 463-468. 10.1038/nature10777.
Bourgon, R., Gentleman, R., and Huber, W. (2010). Independent filtering increases detection power for high-throughput experiments. Proc Natl Acad Sci USA 107, 9546-9551. 10.1073/pnas.0914005107.
Branon, T. C., Bosch, J. A., Sanchez, A. D., Udeshi, N. D., Svinkina, T., Carr, S. A., Feldman, J. L., Perrimon, N., and Ting, A. Y. (2018). Efficient proximity labeling in living cells and organisms with TurbolD. Nat Biotechnol 36, 880-887. 10.1038/nbt.4201.
Chow, L. S., Gerszten, R. E., Taylor, J. M., Pedersen, B. K., van Praag, H., Trappe, S., Febbraio, M. A., Galis, Z. S., Gao, Y., Haus, J. M., et al. (2022). Exerkines in health, resilience and disease. Nat Rev Endocrinol 18, 273-289. 10.1038/s41574-022-00641-2.
Consortium, U. (2021). UniProt: the universal protein knowledgebase in 2021. Nucleic Acids Res 49, D480-D489. 10.1093/nar/gkaa1100.
Contrepois, K., Wu, S., Moneghetti, K. J., Homburg, D., Ahadi, S., Tsai, M. S., Metwally, A. A., Wei, E., Lee-McMullen, B., Quijada, J. V., et al. (2020). Molecular Choreography of Acute Exercise. Cell 181, 1112-1130.e1116. 10.1016/j.cell.2020.04.043.
Cornelissen, V. A., and Smart, N. A. (2013). Exercise training for blood pressure: a systematic review and meta-analysis. J Am Heart Assoc 2, e004473. 10.1161/JAHA.112.004473.
De Miguel, Z., Khoury, N., Betley, M. J., Lehallier, B., Willoughby, D., Olsson, N., Yang, A. C., Hahn, O., Lu, N., Vest, R. T., et al. (2021). Exercise plasma boosts memory and dampens brain inflammation via clusterin. Nature 600, 494-499. 10.1038/s41586-021-04183-x.
De Nardo, W., Miotto, P. M., Bayliss, J., Nie, S., Keenan, S. N., Montgomery, M. K., and Watt, M. J. (2022). Proteomic analysis reveals exercise training induced remodelling of hepatokine secretion and uncovers syndecan-4 as a regulator of hepatic lipid metabolism. Mol Metab 60, 101491. 10.1016/j.molmet.2022.101491.
Dermit, M., Peters-Clarke, T. M., Shishkova, E., and Meyer, J. G. (2021). Peptide Correlation Analysis (PeCorA) Reveals Differential Proteoform Regulation. J Proteome Res 20, 1972-1980. 10.1021/acs.jproteome.0c00602.
Droujinine, I. A., Meyer, A. S., Wang, D., Udeshi, N. D., Hu, Y., Rocco, D., McMahon, J. A., Yang, R., Guo, J., Mu, L., et al. (2021). Proteomics of protein trafficking by in vivo tissue-specific labeling. Nat Commun 12, 2382. 10.1038/s41467-021-22599-x.
Duggan, C., Xiao, L., Wang, C. Y., and McTiernan, A. (2014). Effect of a 12-month exercise intervention on serum biomarkers of angiogenesis in postmenopausal women: a randomized controlled trial. Cancer Epidemiol Biomarkers Prev 23, 648-657. 10.1158/1055-9965.EPI-13-1155.
Endale, M., Ahlfeld, S., Bao, E., Chen, X., Green, J., Bess, Z., Weirauch, M. T., Xu, Y., and Perl, A. K. (2017). Temporal, spatial, and phenotypical changes of PDGFRα expressing fibroblasts during late lung development. Dev Biol 425, 161-175. 10.1016/j.ydbio.2017.03.020.
Finck, B. N., and Kelly, D. P. (2006). PGC-1 coactivators: inducible regulators of energy metabolism in health and disease. J Clin Invest 116, 615-622. 10.1172/JC127794.
   Fukai, T., Siegfried, M. R., Ushio-Fukai, M., Cheng, Y., Kojda, G., and Harrison, D. G. (2000). Regulation of the vascular extracellular superoxide dismutase by nitric oxide and exercise training. J Clin Invest 105, 1631-1639. 10.1172/JC19551.
Goldstein, M. S. (1961). Humoral Nature of the Hypoglycemic Factor of Muscular Work. Diabetes 10, 232-234. 10.2337/diab.10.3.232.
Gombash Lampe, S. E., Kaspar, B. K., and Foust, K. D. (2014). Intravenous injections in neonatal mice. J Vis Exp, e52037. 10.3791/52037.
Green, J., Endale, M., Auer, H., and Perl, A. K. (2016). Diversity of Interstitial Lung Fibroblasts Is Regulated by Platelet-Derived Growth Factor Receptor a Kinase Activity. Am J Respir Cell Mol Biol 54, 532-545. 10.1165/rcmb.2015-00950C.
Hambrecht, R., Walther, C., Möbius-Winkler, S., Gielen, S., Linke, A., Conradi, K., Erbs, S., Kluge, R., Kendziorra, K., Sabri, O., et al. (2004). Percutaneous coronary angioplasty compared with exercise training in patients with stable coronary artery disease: a randomized trial. Circulation 109, 1371-1378. 10.1161/01.CIR.0000121360.31954.1F.
Hanaoka, Y., Yasuda, O., Soejima, H., Miyata, K., Yamamoto, E., Izumiya, Y., Maeda, N., Ohishi, M., Rakugi, H., Oike, Y., et al. (2014). Tissue inhibitor of metalloproteinase-3 knockout mice exhibit enhanced energy expenditure through thermogenesis. PLoS One 9, e94930. 10.1371/journal.pone.0094930.
Hawley, J. A., Hargreaves, M., Joyner, M. J., and Zierath, J. R. (2014). Integrative biology of exercise. Cell 159, 738-749. 10.1016/j.cell.2014.10.029.
Hitomi, Y., Watanabe, S., Kizaki, T., Sakurai, T., Takemasa, T., Haga, S., Ookawara, T., Suzuki, K., and Ohno, H. (2008). Acute exercise increases expression of extracellular superoxide dismutase in skeletal muscle and the aorta. Redox Rep 13, 213-216. 10.1179/135100008X308894.
Hornbeck, P. V., Zhang, B., Murray, B., Kornhauser, J. M., Latham, V., and Skrzypek, E. (2015). PhosphoSitePlus, 2014: mutations, PTMs and recalibrations. Nucleic Acids Res 43, D512-520. 10.1093/nar/gku1267.
Horowitz, A. M., Fan, X., Bieri, G., Smith, L. K., Sanchez-Diaz, C. I., Schroer, A. B., Gontier, G., Casaletto, K. B., Kramer, J. H., Williams, K. E., and Villeda, S. A. (2020). Blood factors transfer beneficial effects of exercise on neurogenesis and cognition to the aged brain. Science 369, 167-173. 10.1126/science.aaw2622.
Hsieh, Y. T., Lin, H. P., Chen, B. M., Huang, P. T., and Roffler, S. R. (2015). Effect of Cellular Location of Human Carboxylesterase 2 on CPT-11 Hydrolysis and Anticancer Activity. PLoS One 10, e0141088. 10.1371/journal.pone.0141088.
Hyatt, H. W., and Powers, S. K. (2020). The Role of Calpains in Skeletal Muscle Remodeling with Exercise and Inactivity-induced Atrophy. Int J Sports Med 41, 994-1008. 10.1055/α-1199-7662. Jiang, Z., Zhao, M., Voilquin, L., Jung, Y., Aikio, M. A., Sahai, T., Dou, F. Y., Roche, A. M.,
Carcamo-Orive, I., Knowles, J. W., et al. (2021). Isthmin-1 is an adipokine that promotes glucose uptake and improves glucose tolerance and hepatic steatosis. Cell Metab 33, 1836-1852.e1811. 10.1016/j.cmet.2021.07.010.
Kanzleiter, T., Wilks, D., Preston, E., Ye, J., Frangioudakis, G., and Cooney, G. J. (2009). Regulation of the nuclear hormone receptor nur77 in muscle: influence of exercise-activated pathways in vitro and obesity in vivo. Biochim Biophys Acta 1792, 777-782. 10.1016/j.bbadis.2009.05.002.
Kawasaki, E., Hokari, F., Sasaki, M., Sakai, A., Koshinaka, K., and Kawanaka, K. (2009). Role of local muscle contractile activity in the exercise-induced increase in NR4A receptor mRNA expression. J Appl Physiol (1985) 106, 1826-1831. 10.1152/japplphysiol.90923.2008.
Kim, K. E., Park, I., Kim, J., Kang, M. G., Choi, W. G., Shin, H., Kim, J. S., Rhee, H. W., and Suh, J. M. (2021). Dynamic tracking and identification of tissue-specific secretory proteins in the circulation of live mice. Nat Commun 12, 5204. 10.1038/s41467-021-25546-y.
Knowler, W. C., Barrett-Connor, E., Fowler, S. E., Hamman, R. F., Lachin, J. M., Walker, E. A., Nathan, D. M., and Group, D. P. P. R. (2002). Reduction in the incidence of type 2 diabetes with lifestyle intervention or metformin. N Engl J Med 346, 393-403. 10.1056/NEJMoa012512.
Knudsen, N. H., Stanya, K. J., Hyde, A. L., Chalom, M. M., Alexander, R. K., Liou, Y. H., Starost, K. A., Gangl, M. R., Jacobi, D., Liu, S., et al. (2020). Interleukin-13 drives metabolic conditioning of muscle to endurance exercise. Science 368. 10.1126/science.aat3987.
Kraemer, W. J., and Ratamess, N. A. (2005). Hormonal responses and adaptations to resistance exercise and training. Sports Med 35, 339-361. 10.2165/00007256-200535040-00004.
Kurgan, N., Noaman, N., Pergande, M. R., Cologna, S. M., Coorssen, J. R., and Klentrou, P. (2019). Changes to the Human Serum Proteome in Response to High Intensity Interval Exercise: A Sequential Top-Down Proteomic Analysis. Front Physiol 10, 362. 10.3389/fphys.2019.00362.
Law, C. W., Alhamdoosh, M., Su, S., Dong, X., Tian, L., Smyth, G. K., and Ritchie, M. E. (2016). RNA-seq analysis is easy as 1-2-3 with limma, Glimma and edgeR. F1000Res 5. 10.12688/f1000research.9005.3.
Lear, S. A., Hu, W., Rangarajan, S., Gasevic, D., Leong, D., Iqbal, R., Casanova, A., Swaminathan, S., Anjana, R. M., Kumar, R., et al. (2017). The effect of physical activity on mortality and cardiovascular disease in 130 000 people from 17 high-income, middle-income, and low-income countries: the PURE study. Lancet 390, 2643-2654. 10.1016/S0140-6736(17)31634-3.
Li, R., Bernau, K., Sandbo, N., Gu, J., Preissl, S., and Sun, X. (2018). marks a cellular lineage with distinct contributions to myofibroblasts in lung maturation and injury response. Elife 7. 10.7554/eLife.36865.
Li, Y., Zalzala, M., Jadhav, K., Xu, Y., Kasumov, T., Yin, L., and Zhang, Y. (2016). Carboxylesterase 2 prevents liver steatosis by modulating lipolysis, endoplasmic reticulum stress, and lipogenesis and is regulated by hepatocyte nuclear factor 4 alpha in mice. Hepatology 63, 1860-1874. 10.1002/hep.28472.
Liu, H., Chen, S. E., Jin, B., Carson, J. A., Niu, A., Durham, W., Lai, J. Y., and Li, Y. P. (2010). TIMP3: a physiological regulator of adult myogenesis. J Cell Sci 123, 2914-2921. 10.1242/jcs.057620.
Liu, J., Jang, J. Y., Pirooznia, M., Liu, S., and Finkel, T. (2021). The secretome mouse provides a genetic platform to delineate tissue-specific in vivo secretion. Proc Natl Acad Sci USA 118. 10.1073/pnas.2005134118.
Lundsgaard, A. M., and Kiens, B. (2014). Gender differences in skeletal muscle substrate metabolism—molecular mechanisms and insulin sensitivity. Front Endocrinol (Lausanne) 5, 195. 10.3389/fendo.2014.00195.
Lynes, M. D., Leiria, L. O., Lundh, M., Bartelt, A., Shamsi, F., Huang, T. L., Takahashi, H., Hirshman, M. F., Schlein, C., Lee, A., et al. (2017). The cold-induced lipokine 12,13-diHOME promotes fatty acid transport into brown adipose tissue. Nat Med 23, 631-637. 10.1038/nm.4297.
Maresch, L. K., Benedikt, P., Feiler, U., Eder, S., Zierler, K. A., Taschler, U., Kolleritsch, S., Eichmann, T. O., Schoiswohl, G., Leopold, C., et al. (2019). Intestine-Specific Overexpression of Carboxylesterase 2c Protects Mice From Diet-Induced Liver Steatosis and Obesity. Hepatol Commun 3, 227-245. 10.1002/hep4.1292.
McGee, S. L., and Hargreaves, M. (2020). Exercise adaptations: molecular mechanisms and potential targets for therapeutic benefit. Nat Rev Endocrinol 16, 495-505. 10.1038/s41574-020-0377-1.
Meex, R. C., Hoy, A. J., Morris, A., Brown, R. D., Lo, J. C., Burke, M., Goode, R. J., Kingwell, B. A., Kraakman, M. J., Febbraio, M. A., et al. (2015). Fetuin B Is a Secreted Hepatocyte Factor Linking Steatosis to Impaired Glucose Metabolism. Cell Metab 22, 1078-1089. 10.1016/j.cmet.2015.09.023.
Mellacheruvu, D., Wright, Z., Couzens, A. L., Lambert, J. P., St-Denis, N. A., Li, T., Miteva, Y. V., Hauri, S., Sardiu, M. E., Low, T. Y., et al. (2013). The CRAPome: a contaminant repository for affinity purification-mass spectrometry data. Nat Methods 10, 730-736. 10.1038/nmeth.2557.
Merrick, D., Sakers, A., Irgebay, Z., Okada, C., Calvert, C., Morley, M. P., Percec, I., and Seale, P. (2019). Identification of a mesenchymal progenitor cell hierarchy in adipose tissue. Science 364. 10.1126/science.aav2501.
Myers, J., Prakash, M., Froelicher, V., Do, D., Partington, S., and Atwood, J. E. (2002). Exercise capacity and mortality among men referred for exercise testing. N Engl J Med 346, 793-801. 10.1056/NEJMoa011858.
Neufer, P. D., Bamman, M. M., Muoio, D. M., Bouchard, C., Cooper, D. M., Goodpaster, B. H., Booth, F. W., Kohrt, W. M., Gerszten, R. E., Mattson, M. P., et al. (2015). Understanding the Cellular and Molecular Mechanisms of Physical Activity-Induced Health Benefits. Cell Metab 22, 4-11. 10.1016/j.cmet.2015.05.011.
Ntokou, A., Klein, F., Dontireddy, D., Becker, S., Bellusci, S., Richardson, W. D., Szibor, M., Braun, T., Morty, R. E., Seeger, W., et al. (2015). Characterization of the platelet-derived growth factor receptor-α-positive cell lineage during murine late lung development. Am J Physiol Lung Cell Mol Physiol 309, L942-958. 10.1152/ajplung.00272.2014.
Oosterhoff, D., Overmeer, R. M., de Graaf, M., van der Meulen, I. H., Giaccone, G., van Beusechem, V. W., Haisma, H. J., Pinedo, H. M., and Gerritsen, W. R. (2005). Adenoviral vector-mediated expression of a gene encoding secreted, EpCAM-targeted carboxylesterase-2 sensitises colon cancer spheroids to CPT-11. Br J Cancer 92, 882-887. 10.1038/sj.bjc.6602362.
Ovens, M. J., Davies, A. J., Wilson, M. C., Murray, C. M., and Halestrap, A. P. (2010a). AR-C155858 is a potent inhibitor of monocarboxylate transporters MCT1 and MCT2 that binds to an intracellular site involving transmembrane helices 7-10. Biochem J 425, 523-530. 10.1042/BJ20091515.
Ovens, M. J., Manoharan, C., Wilson, M. C., Murray, C. M., and Halestrap, A. P. (2010b). The inhibition of monocarboxylate transporter 2 (MCT2) by AR-C155858 is modulated by the associated ancillary protein. Biochem J 431, 217-225. 10.1042/BJ20100890.
Parker, B. L., Burchfield, J. G., Clayton, D., Geddes, T. A., Payne, R. J., Kiens, B., Wojtaszewski, J. F. P., Richter, E. A., and James, D. E. (2017). Multiplexed Temporal Quantification of the Exercise-regulated Plasma Peptidome. Mol Cell Proteomics 16, 2055-2068. 10.1074/mcp.RA 117.000020.
Perez-Riverol, Y., Bai, J., Bandla, C., Garcia-Seisdedos, D., Hewapathirana, S., Kamatchinathan, S., Kundu, D. J., Prakash, A., Frericks-Zipper, A., Eisenacher, M., et al. (2022). The PRIDE database resources in 2022: a hub for mass spectrometry-based proteomics evidences. Nucleic Acids Res 50, D543-D552. 10.1093/nar/gkab1038.
Piercy, K. L., Troiano, R. P., Ballard, R. M., Carlson, S. A., Fulton, J. E., Galuska, D. A., George, S. M., and Olson, R. D. (2018). The Physical Activity Guidelines for Americans. JAMA 320, 2020-2028. 10.1001/jama.2018.14854.
Pino, L. K., Just, S. C., MacCoss, M. J., and Searle, B. C. (2020a). Acquiring and Analyzing Data Independent Acquisition Proteomics Experiments without Spectrum Libraries. Mol Cell Proteomics 19, 1088-1103. 10.1074/mcp.P119.001913.
Pino, L. K., Searle, B. C., Bollinger, J. G., Nunn, B., MacLean, B., and MacCoss, M. J. (2020b). The Skyline ecosystem: Informatics for quantitative mass spectrometry proteomics. Mass Spectrom Rev 39, 229-244. 10.1002/mas.21540.
Potter, P. M., Wolverton, J. S., Morton, C. L., Wierdl, M., and Danks, M. K. (1998). Cellular localization domains of a rabbit and a human carboxylesterase: influence on irinotecan (CPT-11) metabolism by the rabbit enzyme. Cancer Res 58, 3627-3632.
Rao, R. R., Long, J. Z., White, J. P., Svensson, K. J., Lou, J., Lokurkar, I., Jedrychowski, M. P., Ruas, J. L., Wrann, C. D., Lo, J. C., et al. (2014). Meteorin-like is a hormone that regulates immune-adipose interactions to increase beige fat thermogenesis. Cell 157, 1279-1291. 10.10161j.cell.2014.03.065.
Raschke, S., Eckardt, K., Bjorklund Holven, K., Jensen, J., and Eckel, J. (2013). Identification and validation of novel contraction-regulated myokines released from primary human skeletal muscle cells. PLoS One 8, e62008. 10.1371/journal.pone.0062008.
Reddy, A., Bozi, L. H. M., Yaghi, O. K., Mills, E. L., Xiao, H., Nicholson, H. E., Paschini, M., Paulo, J. A., Garrity, R., Laznik-Bogoslavski, D., et al. (2020). pH-Gated Succinate Secretion Regulates Muscle Remodeling in Response to Exercise. Cell 183, 62-75.e17. 10.1016/j.cell.2020.08.039.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47. 10.1093/nar/gkv007.
Robbins, J. M., Peterson, B., Schranner, D., Tahir, U. A., Rienmuller, T., Deng, S., Keyes, M. J., Katz, D. H., Beltran, P. M. J., Barber, J. L., et al. (2021). Human plasma proteomic profiles indicative of cardiorespiratory fitness. Nat Metab 3, 786-797. 10.1038/s42255-021-00400-z.
Roepstorff, C., Steffensen, C. H., Madsen, M., Stallknecht, B., Kanstrup, I. L., Richter, E. A., and Kiens, B. (2002). Gender differences in substrate utilization during submaximal exercise in endurance-trained subjects. Am J Physiol Endocrinol Metab 282, E435-447. 10.1152/ajpendo.00266.2001.
Ross, M. K., and Borazjani, A. (2007). Enzymatic activity of human carboxylesterases. Curr Protoc Toxicol Chapter 4, Unit 4.24. 10.1002/0471140856.tx0424s33.
Ruby, M. A., Massart, J., Hunerdosse, D. M., Schonke, M., Correia, J. C., Louie, S. M., Ruas, J. L., Naslund, E., Nomura, D. K., and Zierath, J. R. (2017). Human Carboxylesterase 2 Reverses Obesity-Induced Diacylglycerol Accumulation and Glucose Intolerance. Cell Rep 18, 636-646. 10.1016/j.celrep.2016.12.070.
Ruegsegger, G. N., and Booth, F. W. (2018). Health Benefits of Exercise. Cold Spring Harb Perspect Med 8. 10.1101/cshperspect.a029694.
Rush, A. J., Trivedi, M. H., Wisniewski, S. R., Nierenberg, A. A., Stewart, J. W., Warden, D., Niederehe, G., Thase, M. E., Lavori, P. W., Lebowitz, B. D., et al. (2006). Acute and longer-term outcomes in depressed outpatients requiring one or several treatment steps: a STAR*D report. Am J Psychiatry 163, 1905-1917. 10.1176/ajp.2006.163.11.1905.
Safdar, A., Saleem, A., and Tarnopolsky, M. A. (2016). The potential of endurance exercise-derived exosomes to treat metabolic diseases. Nat Rev Endocrinol 12, 504-517. 10.1038/nrendo.2016.76.
Sanford, J. A., Nogiec, C. D., Lindholm, M. E., Adkins, J. N., Amar, D., Dasari, S., Drugan, J. K., Fernandez, F. M., Radom-Aizik, S., Schenk, S., et al. (2020). Molecular Transducers of Physical Activity Consortium (MoTrPAC): Mapping the Dynamic Responses to Exercise. Cell 181, 1464-1474. 10.1016/j.cell.2020.06.004.
Sato, S., Dyar, K. A., Treebak, J. T., Jepsen, S. L., Ehrlich, A. M., Ashcroft, S. P., Trost, K., Kunzke, T., Prade, V. M., Small, L., et al. (2022). Atlas of exercise metabolism reveals time-dependent signatures of metabolic homeostasis. Cell Metab 34, 329-345.e328. 10.1016/j.cmet.2021.12.016.
Schranner, D., Kastenmuller, G., Schonfelder, M., Romisch-Margl, W., and Wackerhage, H. (2020). Metabolite Concentration Changes in Humans After a Bout of Exercise: a Systematic Review of Exercise Metabolomics Studies. Sports Med Open 6, 11. 10.1186/s40798-020-0238-4.
Searle, B. C., Pino, L. K., Egertson, J. D., Ting, Y. S., Lawrence, R. T., MacLean, B. X., Villen, J., and MacCoss, M. J. (2018). Chromatogram libraries improve peptide detection and quantification by data independent acquisition mass spectrometry. Nat Commun 9, 5128. 10.1038/s41467-018-07454-w.
Severinsen, M. C. K., and Pedersen, B. K. (2020). Muscle-Organ Crosstalk: The Emerging Roles of Myokines. Endocr Rev 41. 10.1210/endrev/bnaa016.
Shin, S., Pang, Y., Park, J., Liu, L., Lukas, B. E., Kim, S. H., Kim, K. W., Xu, P., Berry, D. C., and Jiang, Y. (2020). Dynamic control of adipose tissue development and adult tissue homeostasis by platelet-derived growth factor receptor alpha. Elife 9. 10.7554/eLife.56189.
Smith, L. M., Kelleher, N. L., and Proteomics, C. f. T. D. (2013). Proteoform: a single term describing protein complexity. Nat Methods 10, 186-187. 10.1038/nmeth.2369.
Smyth, G. K. (2004). Linear models and empirical bayes methods for assessing differential expression in microarray experiments. Stat Appl Genet Mol Biol 3, Article3. 10.2202/1544-6115.1027.
Somineni, H. K., Boivin, G. P., and Elased, K. M. (2014). Daily exercise training protects against albuminuria and angiotensin converting enzyme 2 shedding in db/db diabetic mice. J Endocrinol 221, 235-251. 10.1530/JOE-13-0532.
Steensberg, A., van Hall, G., Osada, T., Sacchetti, M., Saltin, B., and Klarlund Pedersen, B. (2000). Production of interleukin-6 in contracting human skeletal muscles can account for the exercise-induced increase in plasma interleukin-6. J Physiol 529 Pt 1, 237-242. 10.1111/j.1469-7793.2000.00237.x.
Stern, J. H., Rutkowski, J. M., and Scherer, P. E. (2016). Adiponectin, Leptin, and Fatty Acids in the Maintenance of Metabolic Homeostasis through Adipose Tissue Crosstalk. Cell Metab 23, 770-784. 10.1016/j.cmet.2016.04.011.
Stöhr, R., Cavalera, M., Menini, S., Mavilio, M., Casagrande, V., Rossi, C., Urbani, A., Cardellini, M., Pugliese, G., Menghini, R., and Federici, M. (2014). Loss of TIMP3 exacerbates atherosclerosis in ApoE null mice. Atherosclerosis 235, 438-443. 10.1016/j.atherosclerosis.2014.05.946.
Takahashi, H., Alves, C. R. R., Stanford, K. I., Middelbeek, R. J. W., Nigro, P., Ryan, R. E., Xue, R., Sakaguchi, M., Lynes, M. D., So, K., et al. (2019). TGF-β2 is an exercise-induced adipokine that regulates glucose and fatty acid metabolism. Nat Metab 1, 291-303. 10.1038/s42255-018-0030-7.
Tang, S., Beattie, A. T., Kafkova, L., Petris, G., Huguenin-Dezot, N., Fiedler, M., Freeman, M., and Chin, J. W. (2022). Mechanism-based traps enable protease and hydrolase substrate discovery. Nature 602, 701-707. 10.1038/s41586-022-04414-9.
Ting, Y. S., Egertson, J. D., Bollinger, J. G., Searle, B. C., Payne, S. H., Noble, W. S., and MacCoss, M. J. (2017). PECAN: library-free peptide detection for data-independent acquisition tandem mass spectrometry data. Nat Methods 14, 903-908. 10.1038/nmeth.4390.
Tyanova, S., Temu, T., Sinitcyn, P., Carlson, A., Hein, M. Y., Geiger, T., Mann, M., and Cox, J. (2016). The Perseus computational platform for comprehensive analysis of (prote)omics data. Nat Methods 13, 731-740. 10.1038/nmeth.3901.
Uhlen, M., Fagerberg, L., Hallström, B. M., Lindskog, C., Oksvold, P., Mardinoglu, A., Sivertsson, A., Kampf, C., Sjöstedt, E., Asplund, A., et al. (2015). Proteomics. Tissue-based map of the human proteome. Science 347, 1260419. 10.1126/science.1260419.
Wang, T., Xu, Y. Q., Yuan, Y. X., Xu, P. W., Zhang, C., Li, F., Wang, L. N., Yin, C., Zhang, L., Cai, X. C., et al. (2020a). Succinate induces skeletal muscle fiber remodeling via SUCNRI signaling. EMBO Rep 21, e50461. 10.15252/embr.202050461.
Wang, Y., Xu, J., Meyers, C. A., Gao, Y., Tian, Y., Broderick, K., Peault, B., and James, A. W. (2020b). PDGFRα marks distinct perivascular populations with different osteogenic potential within adipose tissue. Stem Cells 38, 276-290. 10.1002/stem.3108.
Warburton, D. E. R., and Bredin, S. S. D. (2017). Health benefits of physical activity: a systematic review of current systematic reviews. Curr Opin Cardiol 32, 541-556. 10.1097/HCO.0000000000000437.
Wei, W., Riley, N. M., Lyu, X., Bertozzi, C. R., and Long, J. Z. (2021). Protocol for cell type-specific labeling, enrichment, and proteomic profiling of plasma proteins in mice. STAR Protoc 2, 101014. 10.1016/j.xpro.2021.101014.
Wei, W., Riley, N. M., Yang, A. C., Kim, J. T., Terrell, S. M., Li, V. L., Garcia-Contreras, M., Bertozzi, C. R., and Long, J. Z. (2020). Cell type-selective secretome profiling in vivo. Nat Chem Biol. 10.1038/s41589-020-00698-y.
Wilkinson, S. B., Phillips, S. M., Atherton, P. J., Patel, R., Yarasheski, K. E., Tarnopolsky, M. A., and Rennie, M. J. (2008). Differential effects of resistance and endurance exercise in the fed state on signalling molecule phosphorylation and protein synthesis in human muscle. J Physiol 586, 3701-3717. 10.1113/jphysiol.2008.153916.
Wrann, C. D., White, J. P., Salogiannnis, J., Laznik-Bogoslavski, D., Wu, J., Ma, D., Lin, J. D., Greenberg, M. E., and Spiegelman, B. M. (2013). Exercise induces hippocampal BDNF through a PGC-1α/FNDC5 pathway. Cell Metab 18, 649-659. 10.1016/j.cmet.2013.09.008.
Wu, C., Jin, X., Tsueng, G., Afrasiabi, C., and Su, A. I. (2016). BioGPS: building your own mash-up of gene annotations and expression profiles. Nucleic Acids Res 44, D313-316. 10.1093/nar/gkv1104.
Wu, J., Ruas, J. L., Estall, J. L., Rasbach, K. A., Choi, J. H., Ye, L., Boström, P., Tyra, H. M., Crawford, R. W., Campbell, K. P., et al. (2011). The unfolded protein response mediates adaptation to exercise in skeletal muscle through a PGC-1α/ATF6α complex. Cell Metab 13, 160-169. 10.1016/j.cmet.2011.01.003.
Xu, Y., Pan, X., Hu, S., Zhu, Y., Cassim Bawa, F., Li, Y., Yin, L., and Zhang, Y. (2021). Hepatocyte-specific expression of human carboxylesterase 2 attenuates nonalcoholic steatohepatitis in mice. Am J Physiol Gastrointest Liver Physiol 320, G166-G174. 10.1152/ajpgi.00315.2020.
Yang, J., Vamvini, M., Nigro, P., Ho, L.-L., Galani, K., Alvarez, M., Tanigawa, Y., Laakso, M., Agudelo, L., Pajukanta, P., et al. (2021). Single-cell dissection of obesity-exercise axis in adipose-muscle tissues. bioRxiv, 2021.2011.2022.469622. 10.1101/2021.11.22.469622.
Yang, L., Vaitheesvaran, B., Hartil, K., Robinson, A. J., Hoopmann, M. R., Eng, J. K., Kurland, I. J., and Bruce, J. E. (2011). The fasted/fed mouse metabolic acetylome: N6-acetylation differences suggest acetylation coordinates organ-specific fuel switching. J Proteome Res 10, 4134-4149. 10.1021/pr200313x.
Zepp, J. A., Zacharias, W. J., Frank, D. B., Cavanaugh, C. A., Zhou, S., Morley, M. P., and Morrisey, E. E. (2017). Distinct Mesenchymal Lineages and Niches Promote Epithelial Self-Renewal and Myofibrogenesis in the Lung. Cell 170, 1134-1148.e1110. 10.1016/j.cell.2017.07.034.
Zhang, S. S., Zhu, L., Peng, Y., Zhang, L., Chao, F. L., Jiang, L., Xiao, Q., Liang, X., Tang, J., Yang, H., et al. (2022). Long-term running exercise improves cognitive function and promotes microglial glucose metabolism and morphological plasticity in the hippocampus of APP/PS1 mice. J Neuroinflammation 19, 34. 10.1186/s12974-022-02401-5.

Zincarelli, C., Soltys, S., Rengo, G., and Rabinowitz, J. E. (2008). Analysis of AAV serotypes 1-9 mediated gene expression and tropism in mice after systemic injection. Mol Ther 16, 1073-1080. 10.1038/mt.2008.76.

### II. Additional Studies

### A. Characterization of Elevated Soluble Wildtype and Mutant CES2 in Mice

The S227A and S230A mutants of CES2A-ΔC and CES2C-ΔC, respectively, show loss of carboxylesterase enzymatic activity compared to wildtype CES2A-ΔC and CES2C-ΔC (FIG. 12A). Wildtype and mutant CES2A-ΔC and CES2C-ΔC reach comparable levels in blood plasma of mice 10 weeks after being transduced with the respective virus (FIG. 12B). Food intake remained unchanged between mutant and wildtype CES2A-ΔC and CES2C-ΔC mice (FIG. 12C).

### B. RHBDD1 Promotes Release of CES2

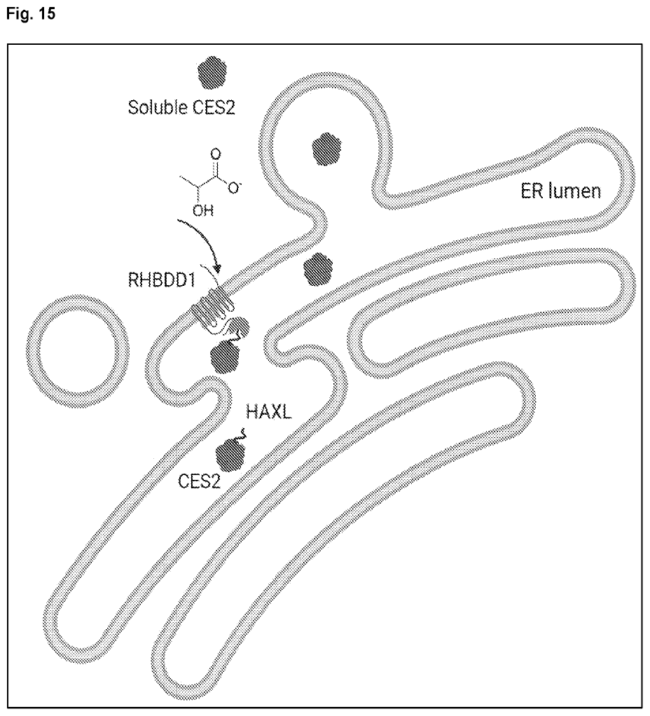

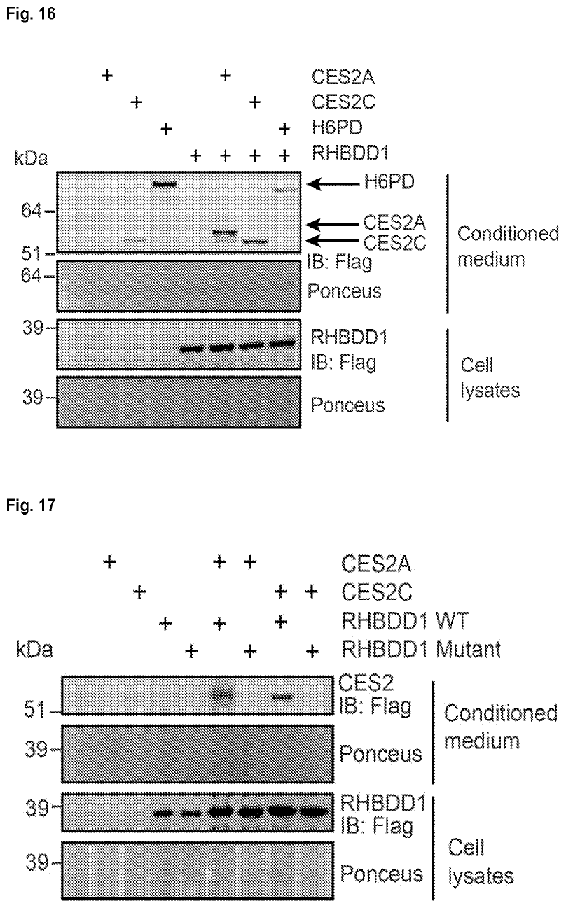

Overexpression of RHBDD1 improves secretion of CES2A and CES2C, but does not affect secretion of H6PD (FIG. 16). Overexpression of mutant (S144A) RHBDD1 lacking protease activity does not improve secretion of CES2A and CES2C (FIG. 17). A model for RHBDD1-mediated CES2 secretion is depicted in FIG. 15.

C. CES2-ΔC does not Induce Canonical Thermogenesis Programs

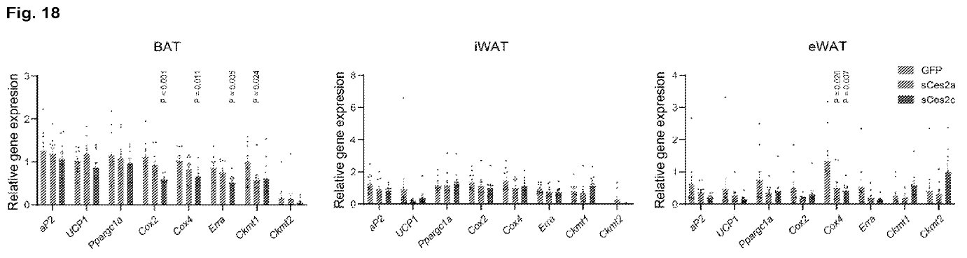

Quantitative PCR was performed to measure the gene expression of canonical thermogenesis program factors across brown adipose tissue (BAT), inguinal white adipose tissue (iWAT), and epididymal white adipose tissue (eWAT) in soluble CES2A-ΔC (sCES2A) and CES2C-ΔC (sCES2C) mice groups compared to GFP mice controls. Decrease in expression of Cox2, Cox4, and Erra were observed in brown adipose tissue for CES2C-ΔC mice compared to GFP mice (P<0.001, P=0.011, and P=0.005, respectively) and decrease in expression of Ckmt1 was observed in brown adipose tissue for CES2A-ΔC mice compared to GFP mice (P=0.024) (FIG. 18). No significant differences in expression were observed in inguinal white adipose tissue for CES2A-ΔC and CES2C-ΔC mice compared to GFP mice (FIG. 18). A decrease in expression of COX4 was observed in epididymal white adipose tissue for CES2A-ΔC and CES2C-ΔC mice, respectively, compared to GFP mice (P=0.020 and P=0.007, respectively) (FIG. 18).

### D. Over-Expression of CES2C-ΔC Induces Lipolysis

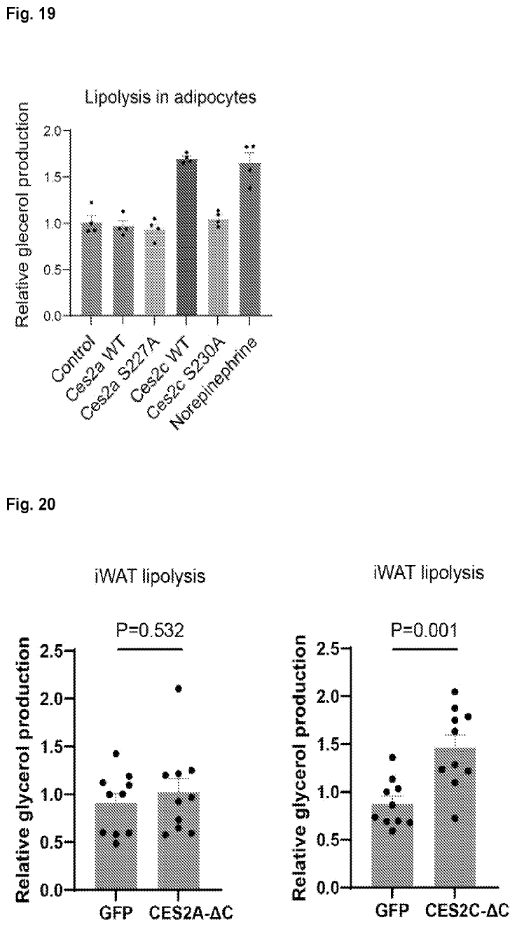

Over-expression of soluble CES2C-ΔC significantly increases glycerol production in differentiated 3T3-L1 cells (adipocytes) compared to mock treated controls and shows comparable glycerol production to norepinephrine treated positive controls (FIG. 19). Over-expression of mutant (S230A) CES2C-ΔC lacking carboxylesterase activity does not increase glycerol production in differentiated 3T3-L1 cells (adipocytes) compared to mock treated controls (FIG. 19).

CES2A-ΔC mice do not exhibit increased glycerol production in inguinal white adipose tissue compared to GFP mice (FIG. 20). CES2C-ΔC mice exhibit significantly increased glycerol production in inguinal white adipose tissue compared to GFP mice (FIG. 20).

### III. Additional Methods Details

a. Mouse models. Animal experiments were performed according to procedures approved by the Stanford University IACUC. Mice were maintained in 12 h light-dark cycles at 22° C. and ˜50% relative humidity and fed a standard irradiated rodent chow diet. Where indicated, high fat diet (Research Diets, D12492) was used. C57BL/6J male mice (stock no. 000664) were purchased from Jackson Laboratory.
b. Cell line cultures. HEK293T cells (CRL-3216) and 3T3-L1 cells (CL-173) were obtained from ATCC and cultured in complete medium (Dulbecco's Modified Eagle's Medium, Corning, 10013CV; 10% FBS, Corning, 35010CV; 1:1,000 penicillin-streptomycin, Gibco, 15140-122). Cells were grown at 37° C. with 5% CO₂. For transient transfection of RHBDD1 WT and RHBDD1 Mutant (S144A), cells were transfected in 10 cm² at ˜60% confluency using PolyFect (Qiagen, 301107) and washed with complete medium 6 h later. 24 h after transfection, cells were washed twice with warm PBS and replaced with serum-free medium. 18 h later, conditioned medium was harvested and concentrated 50 times for Western blotting analysis.
c. Western blotting. For analyzing samples using Western blot, proteins were separated on NuPAGE 4-12% Bis-Tris gels and transferred to nitrocellulose membranes. Equal loading was ensured by staining blots with Ponceau S solution. Blots were then incubated with Odyssey blocking buffer for 30 min at room temperature and incubated with primary antibodies (1:1000 dilution 1:1000 dilution mouse anti-FLAG antibody (Sigma, F1804) in blocking buffer overnight at 4° C. Blots were washed three times with PBST (0.05% Tween-20 in PBS) and stained with species-matched secondary antibodies (1:10000 dilution goat anti-mouse IRDye 680RD (LI-COR, 925-68070)) at room temperature for 1 h. Blots were further washed three times with PBST and imaged with the Odyssey CLx Imaging System.
d. Indirect calorimetry and physiological measurements. 8 to 10-week-old male C57BL/6 mice were injected with AAV8-Tbg-CES2A-ΔC/CES2C-ΔC/GFP viruses via tail vein at a dose of 1011 GC per mouse. One week after viral transduction, mice were fed with HFD (60% fat, Research Diets, D12492). Body weights and food intake were measured every week. Before the body weights of CES2A-ΔC/CES2C-ΔC injected mice started to be significantly different from GFP injected mice (˜3 week for CES2C-ΔC and ˜4 week for CES2A-ΔC), metabolic parameters including oxygen consumption, respiratory exchange ratio, food intake and movement of mice were measured using the environment-controlled home-cage CLAMS system (Columbus Instruments) at the Stanford Diabetes Center. Mice were housed in the metabolic chambers for 24 h prior to the start of experiment. Energy expenditure calculations were normalized for body weight.
e. Quantitative PCR. Inguinal white adipose tissue, epididymal adipose tissues and brown adipose tissues were harvested from 15 to 17-week old male C57BL/6J mice injected with AAV8-Tbg-CES2A-ΔC/CES2C-ΔC/GFP viruses (10e11 GC per mouse) for 7 weeks and fed with HFD (60% fat, Research Diets, D12492) for 6 weeks. Tissues were then homogenized using a Benchmark BeadBlaster Homogenizer at 4° C. The mixture was spun down at 13,000 rpm for 10 min at 4° C. to pellet the insoluble materials. RNA was extracted using a RNeasy Mini Kit (Qiagen, 74106) and reverse-transcribed using a High-Capacity cDNA Reverse Transcription Kit (Thermo Fisher, 4368813). Quantitative PCR was performed using Ssoadvance Universal SYBR Green mix (Biorad, 1725272) with a CFX Opus Real-Time PCR instrument. All values were normalized by the ΔΔCt method to Rps18.
f. Generation of recombinant CES2 proteins. Recombinant CES2A-ΔC WT, CES2A-LC Mutant, CES2C-ΔC WT and CES2C-ΔC Mutant proteins were generated by transient transfection of pDEST40-CES2A/C-LC WT/Mutant plasmids in mammalian Expi293 cells following the manufacturer's instructions. Five to seven days after transfection, conditioned medium was collected, and recombinant proteins were purified using a His GraviTrap TALON column and buffer exchanged to PBS. Protein purity and integrity were analyzed by SDS page. Following purification, recombinant proteins were aliquoted and stored at −80° C. to avoid freeze-thaws.
g. Carboxylesterases enzymatic activity measurement. A continuous spectrophotometric assay was performed using 4-nitrophenyl acetate (Sigma, N8130-5G) as substrate as previously described (Ross and Borazjani, 2007). Briefly, 1 mM 4-nitrophenyl acetate was prepared freshly in 50 mM Tris-CI buffer (pH 7.4). 150 ul of 4-nitrophenyl acetate solution was added into a single well of a 96-well plate (Thermo Scientific, 125565501), followed by pre-incubating 5 min at 37° C. in the absorbance plate reader. 15 ug liver lysates or 3 ul plasma were diluted in 50 mM Tris·Cl buffer (pH 7.4) and added to 4-nitrophenyl acetate solution (total volume 300 ul). The formation of β-nitrophenolate was measured every 30 s at 405 nm for 5 min. Subtracted from background absorbance, absorbance at each time point was used to generate a kinetic plot for each sample. Data points within the linear range of the reaction were used to calculate the slope of the enzymatic reaction. Finally, relative enzymatic activity was calculated comparing the slopes of reaction from each sample.
h. Lipolysis assay in differentiated 3T3-L1 cells. 3T3-L1 cells were cultured in complete medium (DMEM/F-12 Glutamax, Thermo Fisher Scientific #10565018) supplemented with 10% fetal bovine serum and 1% penicillin-streptomycin. Two days post-confluency, differentiation was induced with differentiation medium (complete medium containing 1 mM rosiglitazone, 0.5 mM isobutylmethylxanthine, 1 mM dexamethasone, 5 mg/mL insulin). After two days, cells were replaced with new differentiation medium for additional 2 days. Then fresh complete medium containing 1 mM rosiglitazone and 5 mg/mL insulin was replaced every two days. Cells were fully differentiated after 8 days. On the day of experiment, cells were washed twice with warm PBS. 1 ml of DMEM medium (SF, phenol red free) containing 4% fatty acid-free BSA (Sigma, A7030-100G) and 10 μg of indicated recombinant CES2 proteins were added to each well. 100 nM norepinephrine was used as a positive control for this assay. 3 hours later, 80 ul conditioned medium was collected and added to 150 Glycerol Free Reagent (Sigma, F6428-40ML) in a transparent 96-well plate. Samples were then Incubated at 37° C. for 5 min and glycerol content was read at 540 nm absorbance.
i. Lipolysis assay in inguinal white adipose tissue ex plant. −20 mg of inguinal white adipose tissue from 17 to 19-week old male C57BL/6J mice injected with AAV8-Tbg-CES2A-ΔC/CES2C-ΔC/GFP viruses (10e11 GC per mouse) for 10 weeks and fed with HFD (60% fat, Research Diets, D12492) for 9 weeks were harvested. Body weight and food intake were monitored weekly. Tissues were then incubated in lipolysis buffer (PBS containing 0.1% glucose, 3.5% fatty acid-free BSA (Sigma, A7030-100G), and 2.5 uM norepinephrine) and rotated at 220 rpm for 3 h. 80 ul conditioned medium was then collected and added to 150 Glycerol Free Reagent (Sigma, F6428-40ML) in a transparent 96-well plate. Samples were Incubated at 37° C. for 5 min and glycerol content was read at 540 nm absorbance.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of treating a metabolic disorder in a subject, the method comprising enhancing extracellular CES2 activity in the subject in order to treat the subject for the metabolic disorder, wherein the extracellular CES2 activity is enhanced by administering a CES2 polypeptide lacking a C-terminal ER localization domain.

2.-5. (canceled)

6. The method of claim 1, wherein the metabolic disorder is obesity.

7. The method of claim 1, wherein the metabolic disorder is an obesity-related metabolic disorder selected from the group consisting of overeating, bulimia, hypertension, elevated plasma insulin concentrations and insulin resistance, dyslipidemia, hyperlipidemia, breast, prostate, endometrial and colon cancer, heart disease, cardiovascular disorders, abnormal heart rhythms and arrhythmias, myocardial infarction, congestive heart failure, coronary heart disease, angina pectoris, cerebral infarction, cerebral thrombosis, transient ischemic attack, metabolic syndrome (syndrome X), insulin resistance syndrome, impaired fasting glucose, impaired glucose tolerance, inflammation, systemic inflammation of the vasculature, atherosclerosis, hypercholesterolemia, hyperuricaemia, left ventricular hypertrophy, osteoarthritis, stroke, steatosis, and non-alcoholic fatty liver disease (NAFLD).

8. The method of claim 1, wherein the metabolic disorder is diabetes.

9. The method of claim 1, wherein the method reduces food intake by the subject compared to food intake by the subject before treatment.

10-11. (canceled)

12. The method of claim 1, wherein the method reduces a body weight of the subject compared to a body weight of the subject before treatment.

13. (canceled)

14. The method of claim 1, wherein the method improves glucose homeostasis in the subject compared to glucose homeostasis in the subject before treatment.

15. The method of claim 1, wherein the method improves glucose clearance in the subject compared to glucose clearance in the subject before treatment.

16. (canceled)

17. The method of claim 1, wherein the method reduces adipose tissue mass compared to adipose tissue mass in the subject before treatment.

18-19. (canceled)

20. The method of claim 1, wherein the subject is a mammal.

21. The method of claim 20, wherein the subject is a human.

22.-30. (canceled)

31. An extracellular CES2 activity enhancing CES2 active agent comprising a CES2 polypeptide lacking a C-terminal ER localization domain.

32. The CES2 active agent of claim 31, wherein the CES2 polypeptide comprises an amino acid sequence having 80% or more sequence identity to any one of the amino acid sequences set forth in SEQ ID NOs 1-3.

33. A method of increasing exercise performance in a subject, the method comprising enhancing extracellular CES2 activity in the subject in order to increase exercise performance in the subject.

34. The method of claim 34, wherein the extracellular CES2 activity is enhanced by administering a CES2 active agent to the subject.

35. The method of claim 34, wherein the CES2 active agent is a CES2 polypeptide.

36. The method of claim 35, wherein the CES2 polypeptide lacks a C-terminal ER localization domain.

37. The method of claim 35, wherein the method increases the maximal running speed in a subject compared to a control.

38. The method of claim 35, wherein the method increases the total running time in a subject compared to a control.

39. The method of claim 35, wherein the method increases the total running distance in a subject compared to a control.
